(19) United States
(12) Patent Application Publication
(10) Pub. No.: US 2018/0150650 A1
(43) Pub. Date: May 31, 2018

(54) SYSTEM AND METHOD FOR CONTROLLING PERMISSIONS FOR SELECTED RECIPIENTS BY OWNERS OF DATA

(71) Applicant: The Diary Corporation, Tucson, AZ (US)

(72) Inventors: Kirk L. Saunders, Tucson, AZ (US); Hamish S. MacDonald, Dunedin (NZ); Ryan N. Dickison, Mosgiel (NZ); Michael Van Bokhoven, Auckland (NZ)

(21) Appl. No.: 15/546,940

(22) PCT Filed: Jan. 28, 2016
(86) PCT No.: PCT/US2016/015392

§ 371 Date: Jul. 27, 2017

Related U.S. Application Data
(60) Provisional application No. 62/110,315, filed on Jan. 30, 2015.
(60) Provisional application No. 62/128,830, filed on Mar. 5, 2015.

(51) Int. Cl. G06F 21/62; G06F 17/30; G16H 10/60

(52) U.S. Cl. CPC G06F 21/6245; G16H 15/00; G16H 10/60; G06F 17/30864

(57) ABSTRACT

A method of controlling the granting of permissions to selected recipients by owners of data in a system is described. The method includes identifying permissions that an organization has if an invitation from a particular owner of data is accepted by an administrator of the organization, sending electronic communications comprising at least an invitation from each of the owners and corresponding combinations of permissions to the administrator of the organization, and receiving an assignment of each of the owners to one or more organization-defined access groups. The method further includes receiving an assignment of selected staff members to a plurality of roles, and granting particular permissions to the selected staff members for each of the owners according to one or more access groups having a particular owner and the selected staff members, and to the plurality of roles for the particular owner having the selected staff.

### BACKGROUND OF THE INVENTION

### Field of the Invention

The technology relates to controlling the sharing of owner's data and in particular, providing permissions for selected recipients at a low level of granularity for the data.

### Description of the Related Art

The field of medical records and electronic medical records is widely practiced. Unfortunately, it is also extremely fragmented, with patient data sourced from handwritten and typed notes, voice recordings, and a wide array of computer formats.

Physicians and other healthcare professionals are forced to read multiple pages of data, and mentally build a picture of what is happening, the order of events, and attempting to correlate drug or treatment changes with changes to the patient's physiological measurements or symptoms.

The present art does not provide a way for physicians to see, at a glance, a clear temporally correlated view of the relevant data for a patient.

Problems commonly experienced are: wasted time, greater expertise and training needed to read a patient chart, and errors including, too often, errors that cause serious injury or loss of life.

It has been shown by numerous Health Information Technology software applications and projects that simply coordinating or amalgamating information systems is not necessarily sufficient to improve the quality of information being accessed by clinicians and other healthcare professionals and users. The underlying problem is an entire care team for a patient needs access to a large amount of data but mostly need data to be summarized and presented in more useful ways. If the data is not appropriately summarized and easily understood, a healthcare professional will not be able to utilize it effectively.

Current systems are such that no one sees the whole picture of information. Information is scattered in many places and forms. Frequently data silos exist that prevent accessing or sharing the data. In the healthcare field, many healthcare systems do not communicate with other healthcare systems. Therefore, there is difficulty in accessing a patient's data especially by the patient who can be considered as owning the data. In addition, there are privacy issues related to HIPAA in the United States. Another current difficulty is that different care entities are not on the same page. There is a lack of effective communication between care teams. In conventional medical culture, information flow is top down. Thus, the patient still does not fully understand their health status, and cannot effectively communicate their health story to others. What is desired is a system where the rightful ownership of a patient's health story belongs to the consumer, who can then grant access to doctors, specialists and hospitals.

### SUMMARY

In one aspect, there is a method of controlling the granting of permissions to selected recipients by an owner of data in a system including at least a plurality of computing devices, a server, a network and a database, the method comprising receiving an electronic authentication at the server from an owner of data stored in an owner's electronic diary portion of a database; receiving at the server an electronic address corresponding to each of one or more desired recipients to be invited to access specific categories of data controlled by the owner; identifying of particular permissions that the one or more recipients will have if the invitation is accepted, wherein a permission provides an ability to perform one or more specific actions; sending, via a computer network, an electronic communication to the one or more desired recipients, wherein each electronic communication includes a unique uniform resource locator for use to reply to the communication; receiving, via a computer network, an electronic message including an acceptance or rejection of the invitation from each of the one or more recipients; and storing in a data structure an identifier of each of the one or more recipients, an indicator of the acceptance or rejection of the invitation, and the corresponding combination of permissions granted to each of the one or more recipients if the invitation is accepted so as to facilitate owner-controlled electronic access to the owner's data.

If one of the recipients is an organization, one or more electronic messages may be received identifying one or more particular access groups for the owner and one or more staff members, and one or more roles for the one or more staff members and corresponding categories of data for which the particular permissions are granted. A particular staff member may access a diary only if the owner and the staff member share a common access group. A particular staff member may access a diary based on the cumulative permissions from all roles the staff member belongs to. The particular permissions may be restricted to those given to the organization from the owner's account. A role may be an organization defined combination of permissions. An access group may be an organization defined group to link staff and owners.

The method may additionally comprise receiving an electronic request from the owner to add or remove permissions without the agreement of a particular recipient. The method additionally comprise usage of the granted permissions by the selected recipients comprising receiving a request for a graphical display of owner data from a particular one of the recipients; determining cumulative permissions for the particular recipient, wherein if the particular recipient is a part of an organization, the determining further comprises determining if the particular recipient is associated with an access group shared with the owner; retrieving data identified by the cumulative permissions in the owner's electronic diary; and generating an HTML-based screen display of the retrieved data. The method may further comprise receiving a navigational request from the particular recipient to manipulate the HTML-based screen display. The method may further comprise receiving a navigational request from a particular one of the recipients to manipulate the HTML-based screen display. The screen display may include data for the categories for which a particular one of the recipients has permission, and the screen display may indicate the categories for which the particular one of the recipients does not have permissions. Generating the HTML-based screen display may include applying one or more controls for each category corresponding to the cumulative permissions, wherein the controls for each permitted category may be independent of the controls for the other permitted categories. Generating the HTML-based screen display may include applying one or more predefined templated timeline views based on data from a single category. Generating the HTML-based screen display may include applying one or more predefined templated timeline views based on data from a plurality of categories. If a predefined templated timeline view requires access to data from a category not permitted to the user, the view may not be displayed and user is notified. The method may further comprise receiving a request for a timeline display including an aggregation level selection from among day, week, month or year, and a starting date or ending date corresponding with the aggregation level selection; and rendering a calendar bar having two row portions according to the received request, wherein the bottom row portion displays a series of blocks with dates according to the starting date or ending date corresponding with the aggregation level selection, and wherein the top row portion displays a series of dots at the selected aggregation level, where a light dot represents a lack of owner data for a time period at the selected aggregation level and a dark dot represents the presence of owner data for the time period. The top row portion of the calendar bar may further display a highlighted block over the dots corresponding to the dates in the bottom row portion, and wherein if a cursor is moved to hover over another portion of the top row portion another highlighted block may be displayed corresponding to the position of the hovering cursor, wherein if a click event is received at the position of the hovering cursor, the timeline displays data corresponding to the date of the click and according to the selected aggregation level.

The categories of data may include medications, exercise, sleep, illness and sexual health. The categories of data may include medical records, symptoms and/or vital signs, medications and/or laboratory results, emotional, life events, genetic markers and lifestyle. The recipient may be one of a visitor, staff member, and owner/visitor. The visitor may be one of a guest having read and/or write privileges, and a guardian having full owner privileges. The staff member may be a user who belongs to an organization. The owner/visitor may be a user who is an owner with a diary and also has been given access to another owner's diary. Permission status may include given, received and accepted. Permission kinds may include read, write and no access. The method may additionally comprise receiving an electronic message including a confirmation or cancellation of the received acceptance or rejection of the invitation.

In another aspect, there is a method of controlling the granting of permissions to selected recipients by an owner of data in a system including at least a plurality of computing devices, a server and a network, the method comprising identifying permissions that an organization has if an invitation from a particular owner of the data is accepted by an administrator of the organization, wherein a permission provides an ability to perform one or more specific actions; sending, via a computer network, an electronic communication comprising at least the invitation, the combination of permissions to the administrator of the organization and a unique uniform resource locator for use in replying to the electronic communication; receiving an assignment of the particular owner to an organization-defined access group; receiving a mapping of the particular owner and one or more selected staff members of the organization to the access group to link the particular owner and the selected staff members; receiving an identification of at least one role corresponding to the staff members of the organization; receiving an assignment of the one or more selected staff members of the organization to the at least one role; granting particular permissions to the selected staff members for the particular owner according to the particular access group having the particular owner and the selected staff members, and according to the at least one role having the selected staff; and storing in a data structure an indicator of the acceptance or rejection of the invitation, an identifier of each of the selected staff members and a corresponding combination of permissions granted to each of the selected staff members so as to facilitate owner-controlled electronic access to the owner's data.

A particular staff member may access an owner's diary only if the owner and the staff member share a common access group. A particular staff member may access an owner's diary based on the cumulative permissions from all roles the staff member belongs to. The particular permissions may be restricted to those given to the organization from the owner's account. A role may be an organization defined combination of permissions.

The method may additionally comprise usage of the granted permissions by the selected staff members comprising receiving a request for a graphical display of owner data from a particular one of the staff members; determining cumulative permissions for the particular staff member, wherein the determining further comprises determining if the particular staff member is associated with an access group shared with the owner; retrieving data identified by the cumulative permissions in the owner's electronic diary; and generating an HTML-based screen display of the retrieved data. The method may further comprise receiving a navigational request from the particular staff member to manipulate the HTML-based screen display. The staff member may be a user who belongs to the organization, including one of a doctor, a nurse, a technician, a pharmacist, and an administrator. Permission kinds may include read, write and no access. The roles may additionally include selected categories of data that corresponding staff members have access to, wherein the categories of data include medical records, symptoms and/or vital signs, medications and/or laboratory results, emotional, life events, genetic markers and lifestyle.

In another aspect, there is a method of controlling the granting of permissions to selected recipients by owners of data arranged in owner diaries in a system including at least a plurality of computing devices, a server and a network, the method comprising identifying permissions that an organization will have if an invitation from a particular owner of the data is accepted by an administrator of the organization, wherein a permission provides an ability to perform one or more specific actions; sending, via a computer network, electronic communications comprising at least an invitation from each of the owners and corresponding combinations of permissions to the administrator of the organization, wherein each electronic communication includes a unique uniform resource locator for use in replying to the communication; receiving an assignment of each of the owners to one or more organization-defined access groups; receiving a mapping of a particular owner and one or more selected staff members of the organization to one or more access groups to link the particular owner and the selected staff members; receiving an identification of at least one role corresponding to the staff members of the organization; receiving an assignment of the one or more selected staff members of the organization to the at least one role; granting particular permissions to the selected staff members for each of the owners according to one or more access groups having a particular owner and the selected staff members, and according to the at least one role for the particular owner having the selected staff; and stilling in a data structure an indicator of the acceptance or rejection of the invitation for each of the owners, an identifier of each of the selected staff members and a corresponding combination of permissions granted to each of the selected staff members for each owner so as to facilitate owner-controlled electronic access to each owner's data.

A particular staff member may access a particular diary only if the corresponding particular owner and the staff member share a common access group. A particular staff member may access a particular diary based on the cumulative permissions from all roles the staff member belongs to. The particular permissions may be restricted to those given to the organization from a corresponding owner's account. A role may be an organization defined combination of permissions.

The method may additionally comprise usage of the granted permissions by the selected staff members comprising receiving a request for a graphical display of a particular owner's data from a particular one of the staff members, wherein the data is grouped among multiple categories; determining if the particular staff member is associated with an access group shared with the particular owner; determining cumulative permissions from roles for the particular staff member if the particular staff member is associated with an access group shared with the particular owner; determining which of the cumulative permissions the organization has been granted permission by the owner; retrieving data identified by the cumulative permissions in the particular owner's electronic diary; and generating an HTML-based screen display of the retrieved data. The method may further comprise receiving a navigational request from the particular staff member to manipulate the HTML-based screen display. The screen display may include data for the categories for which the particular staff member has permission, and wherein the screen display may indicate the categories for which the particular staff member does not have permissions. The staff member may be a user who belongs to the organization, including one of a doctor, a nurse, a technician, a pharmacist, and an administrator.

Permission kinds may include read, write and no access. Associated with the roles may be categories of data that corresponding staff members have access to, wherein the categories of data may include medical records, symptoms and/or vital signs, medications and/or laboratory results, emotional, life events, genetic markers and lifestyle, Associated with the roles may be categories of data that corresponding staff members have access to, wherein the categories of data may include body measurements, clinical notes, diary notes, drinking, environment, feelings, immunization, lab results, life events, medication, mood, nutrition, pain, patient stress, physical activity, sleep, smoking, symptoms, treatments and vital signs. Generating the HTML-based screen display includes applying one or more controls for each category corresponding to the granted permissions, wherein the controls for each permitted category are independent of the controls for the other permitted categories. The controls may include choices for sorting, viewing, coloring and filtering the retrieved data. The controls may include a list format and a graphical format for the displaying the retrieved data. Generating the HTML-based screen display may include applying one or more predefined templated timeline views based on data from a single category. Generating the HTML-based screen display may include applying one or more predefined templated timeline views based on data from a plurality of categories. If a predefined templated timeline view requires access to data from a category not permitted to the user, the view may not be displayed and user may be notified.

The method may additionally comprise generating a time-stamped log of the people accessing the owner's data, the action taken by each person, and the changes or additions to the diary, if any.

The method may additionally comprise scanning a source document corresponding to a particular owner; extracting medical data from the scanned document including a reference to the source document; storing the source document in an electronic storage; and storing the extracted medical data and the reference to the source document in a particular diary based on a category of the extracted data, wherein the stored reference enables a user viewing the extracted data to navigate to and view the source document. The source document may be stored with a resolution of a particular web page. The extracted data may be viewed on a timeline or other display page of the data.

The method may further comprise receiving a selection of a data item in a selected category for a particular diary; activating a control in the selected category to initiate a source document link process; displaying a user interface to the electronic storage; receiving a selection by use of the interface of a source document to be linked to the data item; and recording the link for the data item in the particular diary.

The categories of data may include clinical data lifestyle data, psycho-social data, environmental data and genetic data.

In yet another aspect, there is a method of controlling the granting of permissions to selected recipients by an owner of data in a system including at least a plurality of computing devices, a server and a network, the method comprising identifying permissions that an organization has if an invitation from a particular owner of the data is accepted by an administrator of the organization, wherein a permission provides an ability to perform one or more specific actions; sending, via a computer network, an electronic communication comprising at least the invitation, the combination of permissions to the administrator of the organization and a unique uniform resource locator for use in replying to the electronic communication; receiving an assignment of the particular owner to an organization-defined access group; receiving a mapping of the particular owner and one or more selected staff members of the organization to the access group to link the particular owner and the selected staff members; receiving an assignment of the one or more selected staff members of the organization to at least one organizational role; granting particular permissions to the selected staff members for the particular owner according to the particular access group having the particular owner and the selected staff members, and according to the at least one role having the selected staff; storing in a data structure an indicator of the acceptance or rejection of the invitation, an identifier of each of the selected staff members and a corresponding combination of permissions granted to each of the selected staff members; receiving edits to the owner's data or new data from a first user having corresponding granted write permissions; tracking an identity of the first user, a time and date of the edits or new data, a type or category updated, and the edit or new data in a database; and changing a version identifier for the updated owner's data.

The method may additionally comprise determining whether the owner's data has been modified by a second user since it was fetched by the first user; and notifying the first user that the update is disallowed. Determining whether the owner's data has been modified since it was fetched may include determining whether the modified entry is the latest version. The owner's data may be medical or health related data. The update may be visible to each subsequent user having corresponding granted permissions to access the owner's data having the changed version identifier.

The method may additionally comprise displaying a change history for previous versions of the edited owner's data to a user having the appropriate read permission for the category of data corresponding to the edited data.

In another aspect, there is a system for controlling the granting of permissions to selected recipients by an owner of data, wherein the system includes at least a plurality of client computing devices, a server and a database, the system comprising means for receiving an electronic authentication at a server from a client computing device corresponding to an owner of data stored in an owner's electronic diary portion of a database; means for receiving at the server an electronic address corresponding to each of one or more desired recipient client computing devices to be invited to access specific categories of data controlled by the owner; means for identifying of particular permissions that the one or more recipients will have if the invitation is accepted, wherein a permission provides an ability to perform one or more specific actions; means for sending an electronic communication to the one or more desired recipient client computing devices, wherein each electronic communication includes a unique uniform resource locator for use to reply to the communication; means for receiving an electronic message including an acceptance or rejection of the invitation from each of the one or more recipient client computing devices; and means for storing an identifier of each of the one or more recipients, an indicator of the acceptance or rejection of the invitation, and the corresponding combination of permissions granted to each of the one or more recipients if the invitation is accepted so as to facilitate owner-controlled electronic access to the owner's data.

A particular owner of data can have a plurality of client computing devices that correspond to the particular owner.

In another aspect, there is a system for controlling the granting of permissions to selected recipients by owners of data arranged in owner diaries, wherein the system includes at least a plurality of client computing devices and a server, the system comprising means for identifying permissions that an organization will have if an invitation from a particular owner of the data is accepted by an administrator of the organization, wherein a permission provides an ability to perform one or more specific actions; means for sending electronic communications comprising at least an invitation from a client computing device corresponding to each of the owners and corresponding combinations of permissions to the administrator of the organization, wherein each electronic communication includes a unique uniform resource locator for use in replying to the communication; means for receiving an assignment of each of the owners to one or more organization-defined access groups; means for receiving a mapping of a particular owner and one or more selected staff members of the organization to one or more access groups to link the particular owner and the selected staff members; means for receiving an identification of at least one role corresponding to the staff members of the organization; means for receiving an assignment of the one or more selected staff members of the organization to the at least one role; means for granting particular permissions to the selected staff members for each of the owners according to one or more access groups having a particular owner and the selected staff members, and according to the at least one role for the particular owner having the selected staff; and means for storing an indicator of the acceptance or rejection of the invitation for each of the owners, an identifier of each of the selected staff members and a corresponding combination of permissions granted to each of the selected staff members for each owner so as to facilitate owner-controlled electronic access to each owner's data.

A particular owner of data can have a plurality of client computing devices that correspond to the particular owner.

### BRIEF DESCRIPTION OF THE DRAWINGS

In certain embodiments, an owner of data can be healthcare patient. Therefore, where the drawings indicate a patient, it illustrates an instance of an owner.

FIG. 1 is a diagram of an example of an embodiment of a system tool for creating, maintaining and utilizing a Lifetime Health Diary.

FIG. 2A is a diagram of an example of an embodiment of a system configuration of the Lifetime Health Diary tool shown in FIG. 1.

FIG. 2B is a diagram of an example of an embodiment of the electronic storage illustrated in FIG. 2A.

FIG. 2C is a diagram of an example of an embodiment of the interaction between the data vault database, core database and the data vault document storage illustrated in FIG. 2B.

FIG. 3 is a diagram of an example of an embodiment of processing medical information for a particular patient.

FIG. 4 is a diagram of an example of an embodiment of the data sources and structuring portions illustrated in FIG. 3.

FIG. 5 is a diagram introducing permissions for a Diary.

FIG. 6 is a diagram illustrating permissions given from a patient account to an owner/visitor, a guest and a staff member of an organization.

FIG. 7 is a diagram illustrating example parties that the permissions can be given to.

FIG. 8 is a diagram illustrating organizational concepts for the giving of permissions.

FIG. 9 is a diagram illustrating a relationship between staff members and roles.

FIG. 10 is a diagram illustrating a relationship between staff members, access groups and owners.

FIG. 11 is a diagram illustrating staff member access to an owner's account.

FIG. 12 is a diagram illustrating an overview of an embodiment of an invitation process.

FIG. 13 is a flowchart illustrating an embodiment of processing paper documents.

FIG. 14 is a flowchart illustrating an embodiment of linking documents.

FIG. 15 is an example of a screen display illustrating accessing a data vault.

FIG. 16 is an example of a screen display illustrating an upload screen for the data vault.

FIG. 17 is an example of a screen display illustrating user options for the data vault.

FIG. 18 is a diagram illustrating an invitation flow between an owner and a guest.

FIG. 19 is a flowchart illustrating an embodiment of a process for inviting a guest.

FIG. 20 is a flowchart illustrating an embodiment of a process for replying to an invitation.

FIG. 21 is a flowchart illustrating an embodiment of a process for an owner inviting an organization.

FIG. 22 is a flowchart illustrating an embodiment of a process for granting access to a diary.

FIG. 23 is an example of a screen display illustrating a screen for viewing current invitations and for new invitations to be made in the invitations process.

FIG. 24 is an example of a screen display illustrating an interface screen for entering information about a guest and permissions to grant for the guest.

FIG. 25 is an example of a screen display illustrating an example emailed invitation.

FIG. 26 is an example of a screen display illustrating invitation details retrieved such as using the display of FIG. 23.

FIG. 27 is an example of a screen display illustrating an invitation completed screen where the completed invitations can be viewed by the owner and guest.

FIG. 28 is an example of a screen display illustrating an audit of activity by a guest on an owner's account.

FIG. 29 is an example of a screen display illustrating a summary of owners who have given access to their diaries for a particular guest.

FIG. 30 is an example of a screen display illustrating an interface screen seen by a user (e.g., owner) when they wish to update the permissions assigned to a guest.

FIG. 31 is an example of a screen display illustrating an interface screen seen by a user (e.g., owner) when they wish to assign permissions to roles in an organization.

FIG. 32 is an example of a screen display illustrating an interface screen seen by an administrator of an organization when they wish to manage roles in the organization.

FIG. 33 is an example of a screen display illustrating an interface screen seen by an administrator of an organization when they wish to create a role in the organization.

FIG. 34 is an example of a screen display illustrating an interface screen seen by an administrator of an organization of operations that can be performed on corresponding roles in the organization.

FIG. 35 is an example of a screen display illustrating an interface screen seen by an administrator of an organization in managing staff members.

FIG. 36 is an example of a screen display illustrating an interface screen seen by an administrator of an organization to manage role permissions for an organizational role.

FIG. 37 is an example of a screen display illustrating an interface screen seen by an administrator of an organization to manage role permissions for a diary access role.

FIG. 38 is an example of a screen display illustrating an interface screen seen by an administrator of an organization to manage owner groups.

FIG. 39 is an example of a screen display illustrating a screen seen by an administrator of an organization to enter a name and description for a new group.

FIG. 40 is an example of a screen display illustrating an interface screen seen by an administrator of an organization for displaying operations that can be performed on a corresponding group.

FIG. 41 is an example of a screen display illustrating an interface seen by an administrator of an organization to select owners for a corresponding group.

FIG. 42 is an example of a screen display illustrating an interface seen by an administrator of an organization to select staff members for a corresponding group.

FIG. 43 is an example of a screen display illustrating an interface seen by an administrator of an organization for editing a name and description of a corresponding group.

FIG. 44 is a flowchart illustrating an embodiment of a process for determining whether a user interface element is to be rendered.

FIG. 45 is a flowchart illustrating an embodiment of a process for determining whether a particular staff member has access based on permissions.

FIG. 46 is a flowchart illustrating an embodiment of a process for determining whether a particular guest has access based on permissions.

FIG. 47 is an example of a screen display illustrating an interface screen seen by the owner who has set certain permissions as illustrated.

FIG. 48 is an example of a screen display illustrating an interface to display a list of data modules when the owner clicks the plus icon to see to which modules they can add data.

FIG. 49 is an example of a screen display illustrating an interface to display a list of modules restricted to relevant items when a guest performs the same action for the diary of FIG. 48, but their permissions only allow them to enter data for the smoking modules.

FIG. 50 is an example of a screen display illustrating an interface for a pulse page seen by the guest for a diary where only modules for which the guest has view permissions are selectable.

FIG. 51 is an example of a screen display illustrating an interface screen for a timeline which has been chosen but which cannot retrieve data for all modules due to permission limitations and displays a meaningful message informing the user that certain data is not available to them.

FIG. 52 is an example of a screen display illustrating an interface screen for a timeline page showings a time-based summary of an owner's data. The user can choose which modules to view (subject to permissions if the user is not the owner) and each module can have different controls.

FIG. 53 is an example of a screen display illustrating an interface for displaying a page for each module which can be used to work with that module's data.

FIG. 54 is an example of a screen display illustrating a portion of an interface screen seen by a user for navigating a timeline of an owner.

FIG. 55 is an example of a screen display illustrating a calendar bar portion of an interface screen for a timeline showing examples of possible aggregations levels.

FIG. 56 is an example of a screen display illustrating a portion of an interface screen for a timeline showing examples of timeline data maps at a weekly level of aggregation.

FIG. 57 is an example of a screen display illustrating an interface screen for an example widget for physical activity or exercise history showing a list view having two controls.

FIG. 58 is an example of a screen display illustrating an interface screen for an example widget showing a graphical data view responsive to the selected controls including a selected exercise type.

FIG. 59 is an example of a screen display illustrating an interface screen for an example widget showing a graphical data view responsive to the selected controls including all exercise types.

FIG. 60 is an example of a screen display illustrating an interface screen for several example widgets showing a list view responsive to the selected controls and where each widget can have different controls.

FIG. 61 is an example of screen displays illustrating interface screens for example widget settings and widget display for a my health feed widget, and example widget settings and corresponding widget displays for a physical activity widget.

FIG. 62 is an example of screen displays illustrating interface screens for example widget settings and corresponding widget displays for a medication taken widget.

FIG. 63 is an example of screen displays illustrating interface screens for example widget settings and corresponding widget displays for a sleep widget.

FIG. 64 is an example of screen displays illustrating interface screens for example widget settings and corresponding widget displays for a mood widget.

FIG. 65 is an example of screen displays illustrating interface screens for example widget settings and corresponding widget displays for a pain widget.

FIG. 66 is an example of screen displays illustrating interface screens for example widget settings and corresponding widget displays for a body measurements widget.

FIG. 67 is an example of screen displays illustrating interface screens for example widget settings and corresponding widget displays for a vitals widget.

FIG. 68 is an example of screen displays illustrating interface screens for example widget settings and corresponding widget displays for a food and drinks widget.

FIG. 69 is an example of screen displays illustrating interface screens for example widget settings and a corresponding widget display for an access widget.

FIG. 70 is a flowchart illustrating an embodiment of a process for editing and version tracking.

### DETAILED DESCRIPTION OF PREFERRED EMBODIMENTS

### Introduction

The system and method described herein below can be implemented on various configurations of hardware and software. The system can be comprised of various modules, tools, and applications as discussed below. As can be appreciated by one of ordinary skill in the art, each of the modules may comprise various sub-routines, procedures, definitional statements and macros. Each of the modules are typically separately compiled and linked into a single executable program. Therefore, the following description of each of the modules is used for convenience to describe the functionality of the preferred system. Thus, the processes that are undergone by each of the modules may be arbitrarily redistributed to one of the other modules, combined together in a single module, or made available in, for example, a shareable dynamic link library.

The system modules, tools, and applications may be written in any programming language such as, for example, C, C++, C#, BASIC, Visual Basic, Pascal, Ada, Java, HTML, XML, or FORTRAN, and executed on an operating system, such as variants of Windows, Macintosh, UNIX, Linux, VxWorks, or other operating system. C, C++, C#, BASIC, Visual Basic, Pascal, Ada, Java, HTML, XML and FORTRAN are industry standard programming languages for which many commercial compilers can be used to create executable code.

### Definitions

The following provides a number of useful possible definitions of terms used in describing certain embodiments of the disclosed system and method.

Datum: a record of a drug or treatment, symptom, nutrition, lifestyle or lifestyle change, event, mental state, or physiological measurement or condition, comprising a time stamp, a symbol, icon or other means of denoting the event, plus optional fields such as a numerical value, regarding a patient condition or background information.

Data: A plurality of Datum

Data Source: any means of providing data input, including but not limited to a healthcare professional, patient, software program, computer file or program, medical equipment, or sensor, etc., in any format including but not limited to handwritten notes, keyboard notes, images, audio or video recording, electronic file, etc.

Structuring: normalizing a Datum into a consistent and standardized record format.

Tracking: using time stamps or other fields in Structured Data to find, perform calculations on and based on, and graphically display correlations between Data.

Variable: in a clinical trial, treatment change, medication change, or other test or experiment, the Datum which is deliberately varied, while all other Datum are kept invariant (to the extent feasible).

Data Symbol: a graphical icon depicting a Datum, plus optional text or other depiction of additional information including but not limited to time stamp, numerical value, price, etc.

Time Line: a graphical depiction of a plurality of a particular kind of Data Symbols, sorted by their time stamps, or other field including but not limited to medication, or treatment or code number.

Infographic: a graphical or textual depiction of a table, chart, matrix, or other representation of Data Symbols corresponding to Data aggregated from a plurality of patients, sorted by time stamp, or other field including but not limited to age, gender, location, job, lifestyle choices, life events, underlying health conditions including pre-existing conditions, genetic identifier, symptoms, treatments, drugs, or healthcare provider, etc.

Rendering: displaying a plurality of Time Lines or Infographics, each having the same starting and ending time, and time scale.

Analyzing: a process of comprehending and considering the Data or Datum, either in isolation or in correlation or multiple correlations with another Datum or other Data, in order to better understand correlated or causal relationships.

Alert: An Alert is a real-time risk assessment, critical event, reminder, compliance indicator, general indicator, suggestion for medication and/or treatment, referral to a third party, or information pertinent to provision of healthcare. An Alert may include, but is not limited to, any graphical or textual content such as an icon, clock face, VU meter, barometer, thermometer, text, etc.

Message: A Message is the sending of an Alert via network by means including but not limited to, short message service (SMS), instant message, email, tweet, poke, text, automated or manual phone call, video, audio, any form of computer-mediated communication or any other format for sending information, etc.

Routing: Any means of determining appropriate care team members, the patient or other relevant third parties, based on factors including but not limited to, expertise, relationship to the patient, authentication, etc., and delivering a Message to said party or parties.

Network: A network may refer to a network or combination of networks spanning any geographical area, such as a local area network (LAN), wide area network (WAN), regional network, national network, and/or global network. The Internet is an example of a current global computer network. Those terms may refer to hardwire networks, wireless networks, or a combination of hardwire and wireless networks. Hardwire networks may include, for example, fiber optic lines, cable lines, ISDN lines, copper lines, etc. Wireless networks may include, for example, cellular systems, personal communications service (PCS) systems, satellite communication systems, packet radio systems, and mobile broadband systems. A cellular system may use, for example, code division multiple access (CDMA), time division multiple access (TDMA), personal digital phone (PDC), Global System Mobile (GSM), or frequency division multiple access (FDMA), among others.

Website: A website may refer to one or more interrelated web page files and other files and programs on one or more web servers. The files and programs are accessible over a computer network, such as the Internet, by sending a hypertext transfer protocol (HTTP or HTTPS [S-HTTP]) request specifying a uniform resource locator (URL) that identifies the location of one of said web page files, wherein the files and programs are owned, managed or authorized by a single business entity in certain embodiments. Such files and programs can include, for example, hypertext markup language (HTML) files, common gateway interface (CGI) files, and Java applications. The web page files preferably include a home page file that corresponds to a home page of the website. The home page can serve as a gateway or access point to the remaining files and programs contained within the website. In one embodiment, all of the files and programs are located under, and accessible within, the same network domain as the home page file. Alternatively, the files and programs can be located and accessible through several different network domains.

Web page or electronic page: A web page or electronic page may comprise that which is presented by a standard web browser in response to an HTTP request specifying the URL by which the web page file is identified. A web page can include, for example, text, images, sound, video, and animation.

Computer or computing device: A computer or computing device may be any processor controlled device, including terminal devices, such as personal computers, workstations, servers, clients, mini-computers, main-frame computers, laptop computers, a network of individual computers, mobile computers, palm-top computers, hand-held computers, set top boxes for a television, other types of web-enabled televisions, interactive kiosks, personal digital assistants (PDAs), interactive or web-enabled wireless communications devices, mobile web browsers, or a combination thereof. The computers may further possess one or more input devices such as a keyboard, mouse, touch pad, joystick, pen-input-pad, and the like. The computers may also possess an output device, such as a visual display and an audio output. One or more of these computing devices may form a computing environment.

These computers may be uni-processor or multi-processor machines. Additionally, these computers may include an addressable storage medium or computer accessible medium, such as random access memory (RAM), an electronically erasable programmable read-only memory (EEPROM), programmable read-only memory (PROM), erasable programmable read-only memory (EPROM), hard disks, floppy disks, laser disk players, digital video devices, compact disks, video tapes, audio tapes, magnetic recording tracks, electronic networks, and other techniques to transmit or store electronic content such as, by way of example, programs and data. In one embodiment, the computers are equipped with a network communication device such as a network interface card, a modem, or other network connection device suitable for connecting to the communication network such as the Internet. Furthermore, the computers execute an appropriate operating system such as Linux, UNIX, any of the versions of Microsoft Windows, Apple MacOS, IBM OS/2, iOS, Android or other operating system. The appropriate operating system may include a communications protocol implementation that handles all incoming and outgoing message traffic passed over the network. In other embodiments, while the operating system may differ depending on the type of computer, the operating system will continue to provide the appropriate communications protocols to establish communication links with the network.

The computers may contain program logic, or other substrate configuration representing data and instructions, which cause the computer to operate in a specific and predefined manner, as described herein, to be a specialized machine. In one embodiment, the program logic may be implemented as one or more object frameworks or modules. These modules may be configured to reside on the addressable storage medium and configured to execute on one or more processors. The modules include, but are not limited to, software or hardware components that perform certain tasks. Thus, a module may include, by way of example, components, such as, software components, object-oriented software components, class components and task components, processes, functions, attributes, procedures, subroutines, segments of program code, drivers, firmware, microcode, circuitry, data, databases, data structures, tables, arrays, and variables.

The various components of the system may communicate with each other and other components comprising the respective computers through mechanisms such as, by way of example, interprocess communication, remote procedure call, distributed object interfaces, and other various program interfaces. Furthermore, the functionality provided for in the components, modules, and databases may be combined into fewer components, modules, or databases or further separated into additional components, modules, or databases. Additionally, the components, modules, and databases may be implemented to execute on one or more computers. In another embodiment, some of the components, modules, and databases may be implemented to execute on one or more computers external to the website. In this instance, the website includes program logic, which enables the website to communicate with the externally implemented components, modules, and databases to perform the functions as disclosed herein.

### Overview

The system and method described herein is completely owner-centric and facilitates granular user-to-user data sharing. In certain embodiments, the ultimate permission control always remains with the owner or their delegate. A guardian relationship can exist for minors and the incapable. Organizations have the ability to use scalable permission management constructs to enable them to easily and precisely allocate the access they have been given by the owner out to the staff within their organization.

The system and method provides the ability to define granular access where the owner can choose none, read or write access to all areas of their diary of data. Each owner has the ability/visibility to see a list of all users who have the right to potentially view their diary and to see which users have accessed their data. Each owner also has the ability/visibility to see what changes/additions were made when and by which user for their whole diary.

### Description

Embodiments will now be described with reference to the accompanying figures, wherein like numerals refer to like elements throughout. The terminology used in the description presented herein is not intended to be interpreted in any limited or restrictive manner, simply because it is being utilized in conjunction with a detailed description of certain specific embodiments. Furthermore, embodiments may include several novel features, no single one of which is solely responsible for its desirable attributes or which is essential to practicing the inventions herein described.

Some embodiments comprise systems and methods for providing relevant medical data on a chart so that a healthcare provider can quickly understand the history, the correlations of drugs, treatments, nutrition, lifestyle choices, patient physiology values (e.g., blood pressure), and symptoms, including changes to all of these. These systems and methods make it possible for healthcare providers as well as patients and other members of a care team with less training and expertise to competently read and analyze this data. Additionally, it minimizes the risk of errors. In other embodiments, the system and method can be used in other fields where an owner of data desires to control access to the data via permissions.

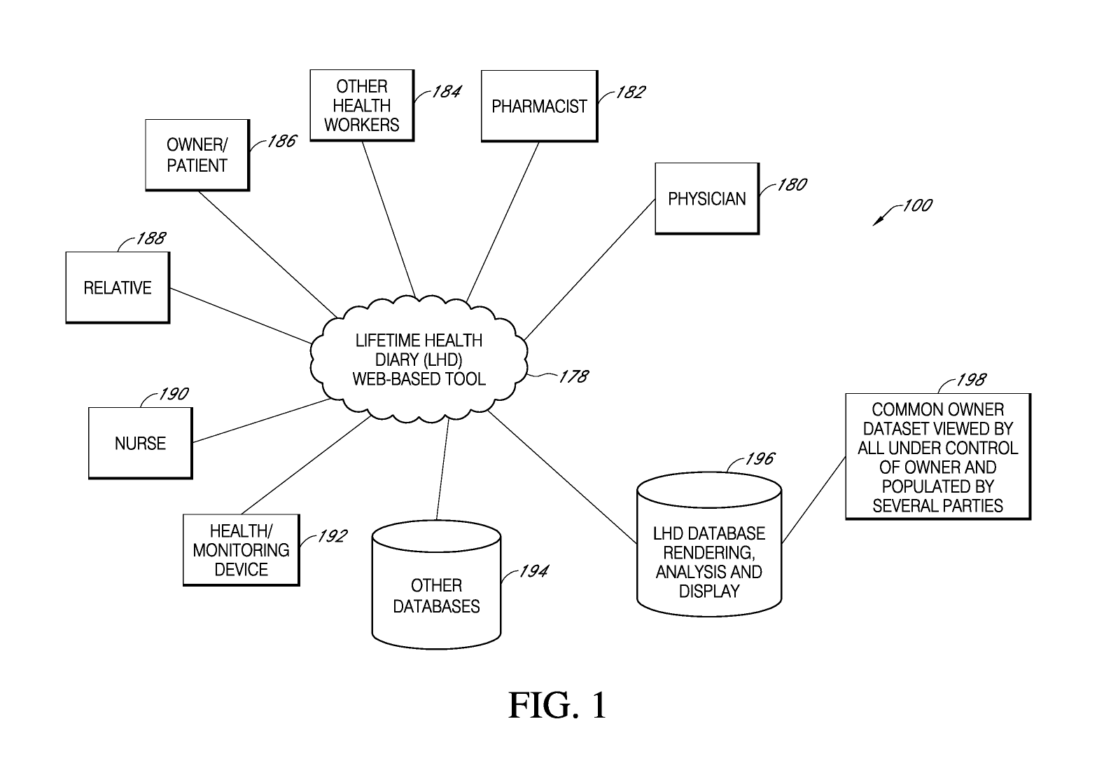

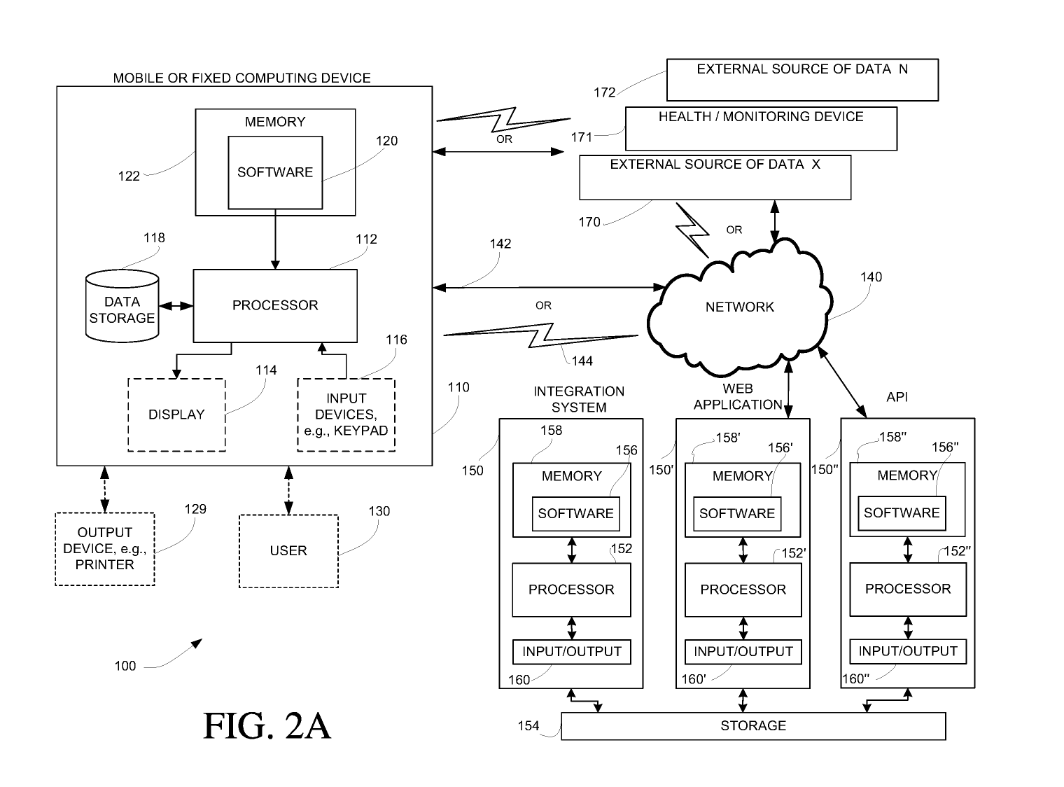

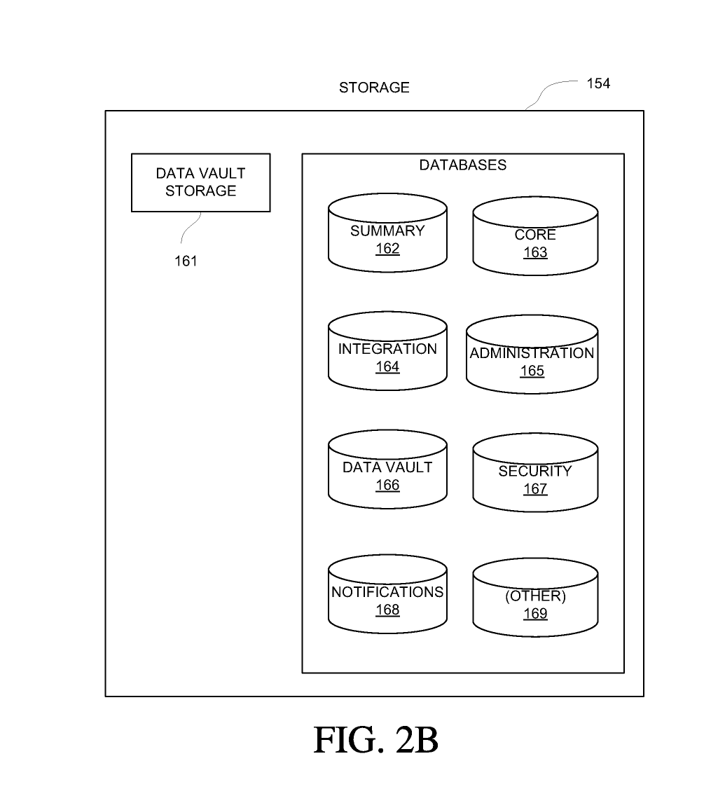

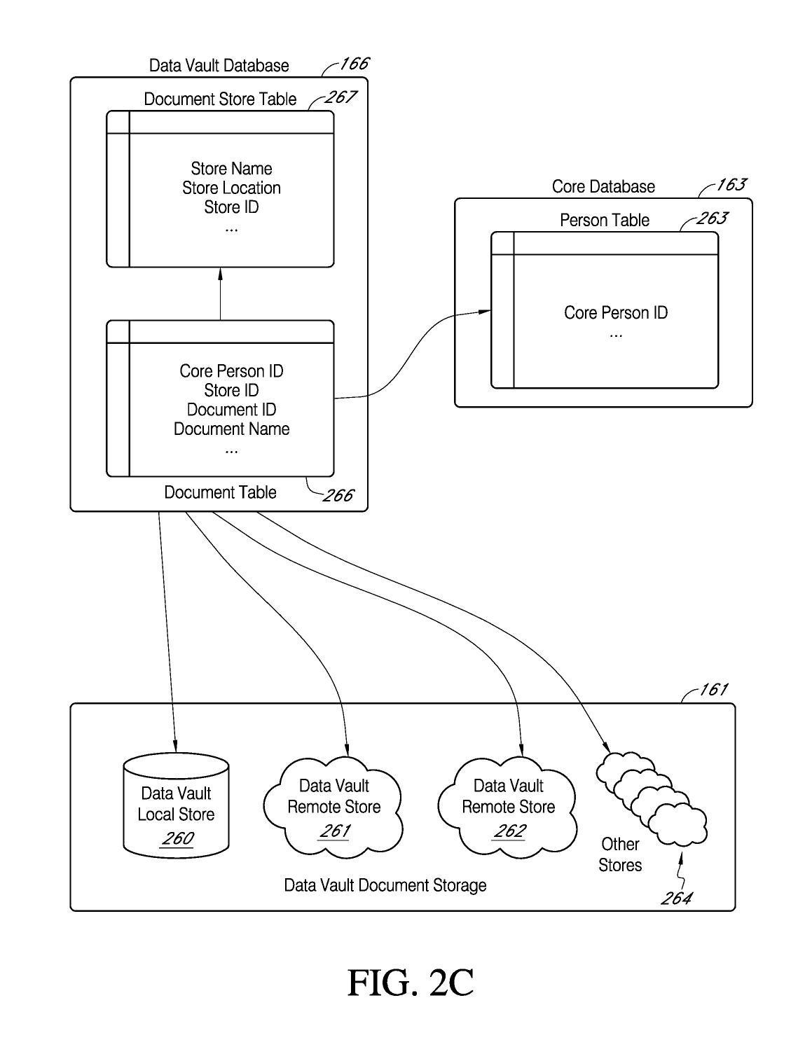

Referring to FIG. 1, an embodiment of a system 100 includes a system tool 178 for creating, maintaining, and utilizing stored medical data pertaining to an individual. Such a collection of data may be referred to as a Lifetime Health Diary (LHD), also referred to as a Diary. Certain embodiments of the system 100 utilize a network as described in conjunction with FIG. 2A hereinbelow. Certain embodiments may utilize a website having web pages to provide access to the tool for one or more of the following parties: an owner or patient 186 (which may include a patient surrogate), a relative 188 (which may also include a family member, a neighbor, a guardian, and so forth), a physician 180 (which may include a primary care physician and/or specialist physician(s)), a nurse 190, a pharmacist 182 (which may include a clinical pharmacist), and other healthcare workers 184 that are not previously enumerated. In certain embodiments, the system 100 includes a LHD database 196 that works with the tool 178 for rendering, analysis, and display of medical information such as a common patient dataset 198 viewed by all parties under control of the patient and populated by several parties. In certain embodiments, the system 100 communicates with one or more health or monitoring devices 192 (e.g., blood pressure monitors, electronic scales), and with other databases 194.

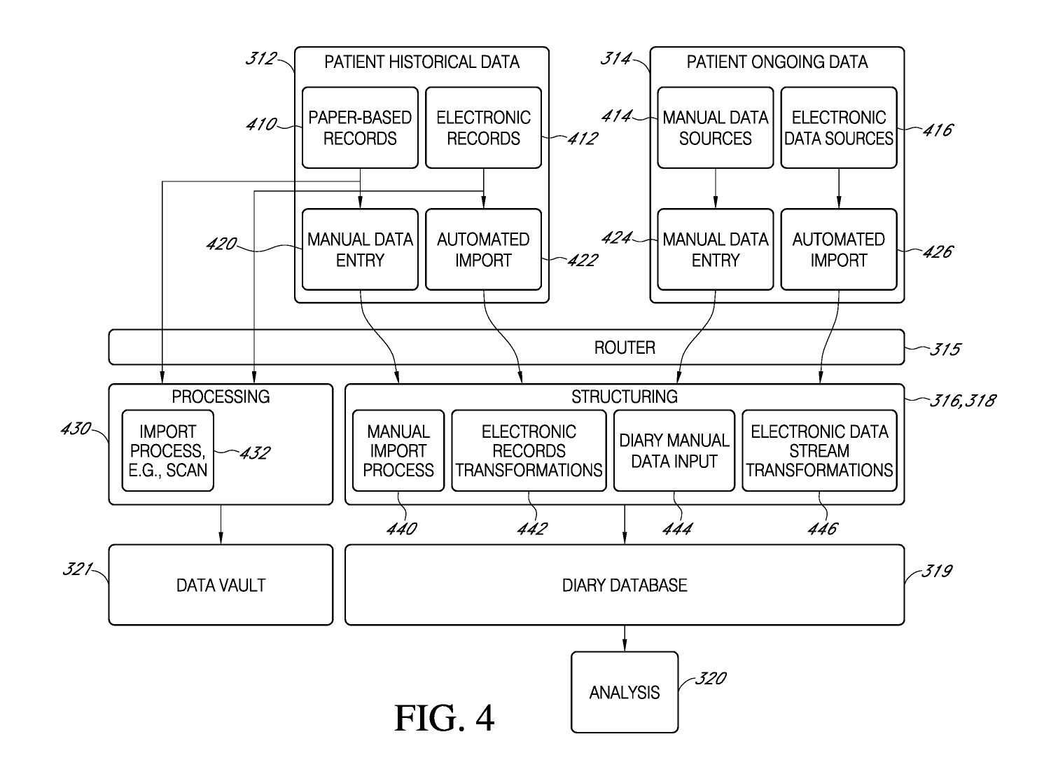

The system 100 may utilize data intermediaries to indicate that the example data sources given (e.g. pharmacist or physician) may not be entering data directly into the Diary, but this data may be brought in via an alternative pathway, e.g., electronically from the physician's or pharmacist's records. Certain embodiments of this will be described below, such as in conjunction with FIG. 4.

Systems and methods for providing relevant medical data on a display in the manner as described herein have many advantages for healthcare providers and patients alike. For example, embodiments of the systems and the methods may provide one or more of the following features and benefits:
Collating data from disparate sources and creating a single database of this medical information allows for easy and rapid analysis of all relevant contextual data.
Converting data into a common structured format and a common data format.
Providing an easy to understand graphic analysis of a patient's overall health.
Providing an easy to understand graphic analysis of a patient's response to a possible drug or a change in their drug regime.
Providing an easy to understand graphic analysis indicating possible contraindications and the likely source of adverse effects. This can include isolating pre-existing patient conditions and complaints from new side effects and/or treatment efficacy that can appear or disappear after starting a particular medication regime.
Alternative forms of health treatment can also be more easily compared to conventional treatment regimes, either for an individual patient, or on a population health basis.
Provides the ability to harness background health information from the patient, or from health professionals involved in patient care. This can be ordered via temporal correlation, enabling direct comparison and correlation with a particular medication regime.
Making it possible to discover relationships between drugs, medical and health data, patient data and professional health opinions of the data.
Reduces inappropriate prescribing.
Reduces medication errors and oversights.
Improves patient adherence, health outcomes and utilization.
Increases cost effectiveness of medication regimes.
Allows easy summation and comprehension of cost of various medication regimes.
Sends easy to comprehend medication regimes, in real-time, to appropriate parties.
Provides an ability to identify correlations between different data types.
Provides an ability for a viewer to rapidly familiarize themselves with the condition/history of the individual (owner) that the diary represents.
Provides an ability for the data display to be easily configured to meet the goals/interests of the current viewer.
Provides an ability to easily/quickly navigate to any specific data at any point in the health history.

These advantages over prior art medical data collection and display systems can be further enhanced because data can be received and displayed in a meaningful manner in real-time. The term “real time” is not used here to denote absolute simultaneous data collection, processing and display. Rather, in certain embodiments real time means that data collection, processing, and display are sufficiently near in time to the actual tracked events to allow treatment decisions to be made in a beneficial manner on an ongoing basis. This will typically allow for conventional time schedules for entering data into health care provider databases and the like, and a frequency of display based on a user's determination of what is suitable for the conditions being monitored. For example, real time may comprise hourly, shift-based, daily, weekly, or monthly updates and/or display viewing depending on the conditions being monitored.

Such a real-time process means that more comprehensive, responsive, and/or cost efficient care (care optimization) can be provided by a health professional to a patient at the point-of-care. In many cases, this may obviate the need to send previously complex and difficult information to a specialist such as a pharmacologist to decipher and interpret, with the corresponding time and expense required.

Embodiments may comprise a health reconciliation tool for collating, analyzing and displaying patient health data from two or more sources at a single point of care. In certain embodiments, the timeline is a temporally correlated, aggregated view of the data that comprises a specific owner diary. The viewport (the currently displayed region) of the timeline can be zoomed in and zoomed out (e.g., day-week-month-year) and also panned back and forward through time. Any subset of the diary data can be selected for viewing and in the order that suits the needs of the viewer.

Certain embodiments are based on an example open system integrated architecture shown in FIG. 1, FIG. 2A, FIG. 2B and FIG. 2C. In FIGS. 2A-2C, the example open system integrated architecture may be based on, for example, a user interface interacting with a local or remote data repository and a local or remote application running on a local or remote application system, such as a web application 150′, application programming interface (web or otherwise) 150″ and integration system 150. In certain embodiments, the web application 150′, application programming interface (API) 150″ and the integration system 150 can each operate on one or more servers, or on one server. FIGS. 2A-2C are block diagrams of an example system 100 that may be used to implement certain systems and methods described herein. The functionality provided for in the components and modules of computing system 100 may be combined into fewer components and modules or further separated into additional components and modules. Various other types of electronic devices communicating in a networked environment may also be used.

A brief overview of some example components is as follows:
Integration system: This system monitors various inbound data pathways, and processes inbound data for addition to an Owner's Diary. For instance, CCDs (Continuity of Care Documents, an XML-based medical history document) can be imported this way, and there is also a format that allows scanned paper documents with annotations to be imported and added to the Diary and Data Vault. This system is for internal use only, and is not directly accessible by any other app.
Notification service: This service monitors the Notifications database, and when notifications occur that need to be sent to third-party systems (e.g., email, SMS), this system will retrieve the data, render the notification text, and send the notification via the requested method. This is an outbound pathway only, and calls messaging systems. It cannot be contacted by any other apps.
Web Application: Commonly called The Diary, this is the website and its backing code that Owners log into via a web browser in order to work with their Diary data, maintain their user profile, invite others to view their Diary, etc.
Admin site: Allows system operator staff to administer the system, including adding/maintaining users, running reports, etc. Not available to non-system operator staff or Owners.
Application Programming Interface: Provides Diary database access to external applications, e.g., an iOS app, an Android app, various test systems. In certain embodiments, there is no access to third party apps, but in other embodiments, there is access to third party apps.
IIS: Microsoft Internet Information Server, which hosts the Diary web app, the admin site, and the API, along with various authentication services in certain embodiments.

The API 150″ provides an interface to which external applications, both third-party and produced by the operator of the system 100, can communicate with the system. It provides a pathway to the databases of storage 154, and functionality that formats data from the databases to make it suitable for consumption by these external clients, and likewise, formats data from the external clients to make it suitable to send to the databases. The API 150″ provides security, such that an external application, and its user (if any), has access only to the data they should have. The API 150″ allows a user or system to update their account, and their medical data, and to retrieve account and medical data, without using the supplied web user interface. In certain embodiments, the API 150″ supports authentication as the owner of the diary data in the API (e.g., a user can't log in as a visitor, for example, and see another owner's diary data). In other embodiments, a user may be able to log in as a visitor and see another owner's diary data with the proper permissions.

Referring to FIG. 2A, an example configuration of components of an embodiment of the system 100 using a network will now be described. In certain embodiments, a mobile or fixed computing device 110 is operated by a user 130. In other embodiments, the computing device 110 can be a server with no explicit user. Such a server can be owned by an operator of the system 100 or that of an integrating third party system, for example. There may be other mobile or fixed computing devices. The computing device 110 can be a handheld computing device or other portable computing device such as a Palm, Pocket personal computer (PC), Linux based handheld, PDA, smartphone such as an iPhone®, Tablet computer such as an iPad®, or PC having a display. In other embodiments, the computing device can be any form of Internet connected device, including but not limited to PCs, mobile devices, PDA, laptops, tablets, chips, keyboards, voice audio and video software, mouse, keypads, touch pads, track ball, microphones, videos, storage devices, network devices, databases, scanners, copiers, digital pens, image recognition software and device, screens and other forms of displays, netbooks and other forms of computer hardware. In certain embodiments, a particular user can have and use multiple computing devices that correspond to the user. In such a multiple user device situation, the system 100 can synchronize the data among each device corresponding to the particular user. The computing device 110 in certain embodiments operates in a stand-alone (independent) manner, such as when it is a server, for example. In other embodiments, the computing device 110 is in communication with the web application 150′ and/or the API 150″ via a network 140. In other embodiments, other numbers of servers can be utilized. The servers can include one or processors 152, memory 158, system software 156 executed by the processor(s), and input or output devices 160. In certain embodiments, a data storage subsystem 154 is in data communication with the integration system 150, web application 150′ and API 150″ and stores one or more databases used by the system, such as the LHD database 196 (FIG. 1). The processor 152′ can be in communication with the database(s) via a database interface, such as structured query language (SQL) or open database connectivity (ODBC). In certain embodiments, the data storage 154 is in data communication with the servers via the database interface. The connection from the computing device 110 to the network 140 can be a wireless or a satellite connection 144 or a wired or direct connection 142. The servers on which the integration system 150, the web application 150′ and the API 150″ operate together with the system software and the data perform as a specialized machine. In certain embodiments, the servers are part of a web site, such as on an intranet or the Internet.

When the computing device 110 is connected with the servers, the web site may optionally provide updates on new features. In another embodiment, the computing device runs only when connected to the servers.

The computing device 110 includes a processor 112, memory 122, a display 114, and one or more input devices 116. When operating as a server, the display 114 and input devices 116 can be optional. The processor 112 is in data communication with a data storage 118. In certain embodiments, the data storage 118 may store records of the user and/or other data or software. System software 120 is executed by the processor 112. The system software 120 may include an application graphical user interface (GUI). The application GUI can include a database interface to the data storage 118 of the computing device. In certain embodiments, the software is loaded from the data storage 118. In certain embodiments, the software can be a mobile application. In embodiments where the computing device 110 communicates with a web site, the processor utilizes browser software in place of or in addition to the software 120. The network browser may be, for example, Microsoft Internet Explorer®, Apple Safari®, Mozilla Firefox®, Google Chrome™, browsers from Opera Software™, and so forth. The computing device 110 together with the system software and the data operates as a specialized machine. An optional output device 129, such as a printer is connected to the computing device 110.

An example of a mobile application includes a Diary iOS application. The Diary iOS application may include a three-way synchronization between the application, the LHD database 196 and a HealthKit framework.

External source of data X 170, a health or monitoring device 171, and external source of data N 172 communicate with wired or wireless connections to the network 140 and/or one or more of the computing devices 110. External sources of data include but are not limited to clinics, hospitals, healthcare networks, insurance, pharmaceuticals, pharmacies, regional health boards, pharmacy benefit managers, population health entities, government and private institutions, paramedics, researchers, health coaches, pharmacologists, physicians and other health professionals, patient networks, educational institutes, employers, laboratories, traditional complementary and alternative medicine practitioners, pharma, clinical research organizations, remote medicine providers, allied health organizations, care givers and care giver organizations. One or more of the external devices 170, 171, 172 may make use of the API 150″ to synchronize data, or may do so via the computing device 110. The external sources of data 170, 172 can include host hardware, which in certain embodiments, uses either a completely redundant hardware infrastructure (e.g., parallel servers or load balancing swap servers) to deliver availability; or gain scalability for its data systems by implementing a multi-processor system for its active system and another multi-processor as a passive standby system. The external sources of data 170, 172 can also include operating systems (e.g., multiprocessing, multi-user, multitasking, and real-time) to provide a software platform on top of which the external entity's application programs can run and ensure that different programs and users running at the same time do not interfere with each other. The operating system is also responsible for security, such as ensuring that unauthorized users do not access the external sources of data 170, 172. The external sources of data 170, 172 can also include a database, such as a relational database from Oracle Corporation. A relational database securely consolidates information and ensures data quality, provides always-available access, scales to deliver the response times users demand, reduces downtime, automates administrative tasks and reduces operational costs through scalability. The external sources of data can push processed or unprocessed medical data which needs further processing to the server(s) associated with the integration system and web application for processing of the medical data. The processed data is used to update the system LHD database. For example, the integration system 150 can consume data that is provided by the system internal data pathways, and writes to the database. From there, client devices can consume the data via the web app, or the API.

Referring to FIG. 2B, an example configuration of components of an embodiment of the storage subsystem 154 will now be described. The storage subsystem 154 can include a data vault storage 161 and a set of databases including a summary database 162, a core database 163, an integration database 164, an administration database 165, a data vault database 166, a security database 167, a notifications database 168 and other databases 169. The data vault storage 161 is a bulk data store for the data vault, which can be a single directory on disk, or a third-party bulk data repository. In certain embodiments, the data vault storage 161 does not need to be indexable or searchable other than by simple filename. The data vault database 166 is a local database that relates data stored in the data vault storage 161 to the owner users in the diary, and holds other metadata such as tags, created dates and so forth.

The summary database 162 holds aggregated data over time for each diary category or module, for consumption by the timeline, and in the future, other systems as required. The core database 163 contains all core data, including users/logins, diary data and so forth. The integration database 164 contains data specific to integration with external systems, e.g., recording state of incoming data. The administration database 165 holds the login and role data for administrative users (e.g., support). The data vault database 166 contains metadata for the data vault documents, but does not store actual binary data. The security database 167 contains assistant stored procedures and in the future, perhaps related tables, for application security. The notifications database 168 contains notifications and metadata for users, e.g., emails that are about to be sent or have been sent, on-screen (web) notifications, SMS notifications and so forth. The notifications database 168 is used by a notifications service to send notifications, by the core application to queue notifications for sending, and by the core application to show on-screen notifications.

The core database 163 can contain all core data, including users/logins, diary data and so forth. In certain embodiments, the core database 163 can store:
Owner-specific diary data, e.g., exercise, smoking, and sleep records.
Static data referred to by these, e.g., master lists of different types of exercise, medications, conditions.
Log-in credential data
Personal/profile data (name, address, social security number, . . . )
Permissions—definitions of permissions, plus permissions for visitors/organizations/roles/ . . . , invitations

In certain embodiments, the summary database 162 can be entirely derived data. The data can be dropped at any time, as it can be recreated on demand. In certain embodiments, it is purely a cache, which avoids recalculating large amounts of data. This calculated data represents groups of diary entries over time. For instance, it may contain a summary of an owner's exercise activity on a daily, weekly, monthly, and yearly basis.

The summary database 162 can hold aggregated data over time for each diary category or module, for consumption by the timeline, and in the future, other systems as required. This consists of records for each module, reflecting data held in that module for periods such as Daily, Weekly, Monthly, and Yearly. Each record consists of at least an Index. This index indicates which type of time period is being summarized, and may contain directly the summary data, or may have sub-records that contain the summary data, depending on the requirements of the module in question. For example, a Sleep module may require only an index, as there is no concept of multiple types of sleep. However, a Symptoms module may need an index (to provide a record on the period being aggregated), and multiple summary sub-records, one to summarize each type of symptom that has occurred during that period.

The Summary database 162 data is built from the core database 163. A summary index is the central entity for each module as follows:
[Id] [int] IDENTITY(1,1) NOT NULL: The ID of the index record.
[PatientId] [int] NOT NULL: The ID of the Owner in the Core database to whom the data belongs.
[PeriodTypeId] [int] NOT NULL: The type of period this summary covers (see below).
[StartDate] [date] NOT NULL: The date on which the period begins.
[EndDate] [date] NOT NULL: The date on which the period ends.
[Count] [int] NOT NULL: The number of records that contributed to this aggregation.

Period types are:
Id
Value
1 Daily
2 Weekly
3 Monthly
4 Yearly

Each index table may have more columns specific to that data type, or there may be a many to one relationship between a module-specific table and its index. For example, a Symptom's index table has only the columns listed above, but it has a sub-table, Symptom Summary:
[Id] [int] IDENTITY(1,1) NOT NULL: The ID of this summary.
[SymptomIndexId] [int] NOT NULL: The symptom index (above) that this summary belongs to.
[SymptomId] [int] NULL: The symptom that is being summarized. For instance, in a single month period, several symptoms may occur. Each is summarized independently, so one may know that one's headaches were severe this month, but ones back pain was much improved.
[Description] [nvarchar](255) NULL: A plain text description of the symptom.
[MinSeverity] [int] NOT NULL: The minimum severity of this symptom that was recorded during this period.
[AvgSeverity] [int] NOT NULL: The average severity of this symptom during this period.
[MaxSeverity] [int] NOT NULL: The maximum severity of this symptom that was recorded during this period.
[RecordCount] [int] NOT NULL: The number of instances of this symptom that were recorded during this period.

However, some modules don't have a concept of different data types that can occur during a period. For example, Stress is simple—any point in time has a level of stress, there's no distinction between kinds of stress. So, the index table can look like the following:

Standard fields as in the index above:
[Id] [int] IDENTITY(1,1) NOT NULL
[PatientId] [int] NOT NULL
[PeriodTypeId] [int] NOT NULL
[StartDate] [date] NOT NULL
[EndDate] [date] NOT NULL
   and fields with similar purpose to the summary above:
[Max] [int] NOT NULL
[Min] [int] NOT NULL
[Average] [int] NOT NULL
[RecordCount] [int] NOT NULL

In certain embodiments, the data vault database 166 contains metadata for the data vault documents and includes the following fields:
UID: a GUID that uniquely identifies the document
FileName: The user-friendly filename of the document
FileSize: The size of the document, in bytes
PersonId: Identifies the owner of this document by their ID (from the Core database)
CreatedDate: The date on which this document was first uploaded
ModifiedDate: The date of the last modification of this document
Notes: Free text notes
DataSourceId: Identifies the origin of this document, e.g., web app, API (mobile apps), integration system
DataSourceRef: A free text field available for use by external apps
EncryptionType: The type of encryption used on this document (currently active for flat files on disk)

In addition there are tags stored in a many to many relationship with documents:
UID: a GUID that uniquely identifies the tag
Name: Text name of the tag
PersonId: Identifies the owner of this tag by their ID (from the Core database)

The integration database 164 contains data specific to integration with external systems, e.g., recording state of incoming data. It details the kinds of integration that are currently supported, and refers the integration system 150 to the code that corresponds to each integration type. In this way, integration pathways can be started and stopped via the database. It records logs of all integration actions, and stores integration messages that are used in the integration pipeline.

The administration database 165 holds the login and role data for administrative users (e.g., support). This is used to limit which areas of the administration web application each admin user is allowed to view/use.

In certain embodiments, the security database 167 contains only assistant stored procedures for application security (permissions). It contains no tables or data in other forms. In other embodiments, it may contain permissions-related tables.

The notifications database 168 can contain notifications and metadata for users, e.g., emails that are about to be sent or have been sent, on-screen (web) notifications, SMS notifications and so forth. The notifications database 168 is used by a notifications service to send notifications, by the core application to queue notifications for sending, and by the core application to show on-screen notifications.

The notification database 168 can contain both templates that are used to create notifications, and the created notifications. Notification templates are as follows. The system is able to generate multiple types of notifications, currently:
Password Reset
New Owner Confirm Email
New Owner Welcome
New Guest Confirm Email
New Guest Welcome
New Account Email Exists
Invite New Guest
Invite New User
Invitation Sent
Invitation Accepted
Invitation Declined
Permissions Modified Guest
Permissions Modified Owner
Account Field Modified
Guest Removed
Guest Removed Other Guest
Guest Removed Self
Guest Invited Other Guest
Person Add New Email
Guest Accepts Invitation
New Free Owner Welcome

New types of notifications can be added regularly. Each notification type has one or more templates, each one representing the content in a given language. Then, each template has multiple content types, e.g., plain text, HTML. In this way, by selecting the type of notification that must be sent, the system is able to send a notification to that user in their own language. Templates are immutable; if the content must be updated, a new set of records is added. This allows old notifications to be regenerated exactly as they appeared when originally sent.

Notification instances are as follows. When the system generates a notification, it is stored as a set of parameters, and a reference to a template. This way, large amounts of duplicate boilerplate text are not, stored in the database in certain embodiments.

Notification distribution is as follows. The notification database 168 can store the status of each notification:
Unsent (waiting for send)
Retrying
Sent
Failed

This is updated by the notifications Windows service and other notifications consumers as required.

Notification template authoring tools are as follows. The notification system includes tools to make it easier for administrators to update notifications. In the admin site, the admin user can modify notification templates. The database holds sample values for template parameters, so that the notifications system can render notifications from the template being updated, to provide the user with a realistic example of their notification.

The system includes the ability to spread the Data Vault data across multiple backing stores. In certain embodiments, the data is spread across local (flat files on disk) storage and a bulk data storage provider, e.g., Amazon AWS S3, but the system is designed to be able to support as many stores as required. Previously, the Data Vault stored its files only on a local or network share drive. This was not workable in the longer term as it can be inflexible, expensive, and required the application server (web or API) to do a lot of work when uploading or download (e.g., encryption). Third party services exist for bulk data storage, so being able to make use of those behind the scene services provides advantages, as all network traffic and other overhead related to the storage of the data can be offloaded from the Diary servers to the storage provider's servers. Other advantages include:
The system can handle multiple backing stored at once. When a user chooses to upload a document, the system can make a decision on which provider to use, and which site.
Multiple service levels are supported. For example, a premium service can be offered where the backing store for those users can be faster, or more secure, or provide other access methods for the data.

The Data Vault database 166 contains a list of currently supported backing stores, in a table called DocumentStore. The Data Vault database Document table has a StoreId column that is foreign keyed to the store table. In the remote backing store, the file is stored by environment (e.g., the Diary system whose Data Vault this is) and user (by URN, as described below), with the original filename being maintained. This way, when a user downloads a file directly from the backing store, the filename of the downloaded document is correct.

In certain embodiments, within a Diary instance, database entities are identified solely by an integer ID. These are not unique even within their database—they only have meaning within the context of a particular table. This is not a significant limitation. However, if there's a need to be able to identify an entity uniquely across multiple Diary systems, this can a problem because each system is likely to have their own local version of entity with id 1, entity with id 2, etc. An example of this is seen in an authentication system implementation by the operator of the system 100, which can be Lifetime Health Diary in some embodiments. In certain embodiments, a Thinktecture IdentityServer3 (OAuth) based authentication system can be utilized. When a user logs on, the authentication system implementation must be able to tell not only what the integer ID of this user's entity is, but also which Diary instance that user's entity (and data) exists within. For that reason, a standard notation to specify the exact location of any given database row/entity has been defined. When writing code within the Diary, one will generally not need to make use of entity uniform resource names (URNs). But if one ever may need to reference an entity on a global basis, entity URNs can be used to do it. An EntityURNService and associated interface have been implemented.

Entity URNs follow the format: urn:<instance>:<db>:<typename>-<id>

| URN segment | Description | Example |
|---|---|---|
| env | The Diary instance in which this entity exists | ins2 |
| db | The database in which this entity is stored | core |
| typename | The type of the entity | Person |
| id | The integer ID of this entity | 44 |

The examples in the table above would result in the URN urn:ins2:core:Person-44.

Referring to FIG. 2C, an example of an embodiment of the interaction between the data vault database, core database and the data vault document storage illustrated in FIG. 2B will be described. The Data Vault database 166 includes a Document table 266 having a Core Person ID, a Store ID, a Document ID, a Document Name and other data/columns/fields. The Data Vault database 166 also includes a DocumentStore table 267 having a Store Name, a Store Location, the Store ID and other data/columns/fields. The Core database 163 includes a Person table 263 having a Core Person ID and other data/columns/fields. The Document table 266 contains fields that identify a document's owner, and its exact storage location. The Core Person ID field denotes the user in the Person table 263 in the Core DB 163 to whom a specific document belongs. The Store ID, Document ID and Document Name fields define the location at which the document can be found. The Store ID indicates in which store (Data Vault local store 260, Data Vault remote store one 261, Data Vault remote store two 262, and further stores represented by 264) the document can be found. The location of the file in the store can be defined by some combination of the Document ID, Document Name, or other metadata derived from the record in the Document table 266. The mapping of a Document table record to a location in a store is defined on a per-store basis, so the scheme may differ from store to store.

In certain embodiments, the Data Vault writes documents to the Amazon AWS S3 data store. However, in other embodiments other factors to choose between available storage locations can include:
Local legal requirements
Business requirements
Security requirements
Geography (physical/network proximity)
User account type—e.g., free, standard, premium accounts may provide different levels of storage.

To read documents, the Data Vault will either proxy the download to the calling client (for API), or provide a download URL, either by redirect or as a response to a specific request.

A database entity can be identified in three ways:
Integer ID identifies entities locally only, within a specific database instance. For instance, a Person with the id 44 may exist in both production instances (an iOS/free account database, and a paid account database). Specifying the integer ID only will not indicate which database the entity can be found in.
UID is globally unique, so there is no ambiguity for an entity identified in this way. However, there is still no way of telling from a UID alone in which database a given entity may be found.
URN is a multi-part identifier, which specifies in full the location of the entity, e.g., which system, which instance, which type, and which record is being requested. This is entirely unambiguous, and provides a complete path to the entity, but may not perform as well as the other two ID types.

Some entities can require the use of all three ID types. For instance, a Person can use integer IDs to enforce referential integrity within the database, a UID to identify it as a target for an integration import, and a URN when referred to by the global authentication system.

### Presentation of Medical Information

Typically, patient medical information is collected using XML data export files into a comma separated values (CSV) file, and then manually displayed in a spreadsheet. Different views of such a spreadsheet makes temporal correlation extremely challenging as the dates can be out of order if sorted by drug name, as are the dosages and any change in medication. On the other hand, other sort orders will mean that drug names are jumbled up, making the view even more confusing. The overall effect of the state of the art is to make decisions and judgment time consuming, non-intuitive, complicated, and usually requiring specific expertise. By contrast, the system 100 displays formats that provide relevant data in a manner that is especially intuitive and helpful for all care team members.

In terms of data collection, data may be pulled as XML data export files (or any other suitable format) into the system that structures and displays them. Medical data may be imported from a variety of sources. These sources include systems that gather or store data about patients today such as paper records, voice recordings, computerized electronic medical records, or that might be developed in the future such as a nano-machine implanted in a patient's body and which uses a wireless communications method to periodically send physiology measurements. The data is structured so that it is put into a consistent computer record structure, with consistent fields, consistent units for values (e.g., grams), and so forth.

The data may be displayed using a consistent set of symbols to denote physiological measurements, drugs, dosages, starts and stops, and so forth. In certain embodiments, a series of vertically aligned horizontal lines are drawn, beginning at the start time (which may be the first time for which data is available, or any other time selected by the user), and ending at the end time (which may be the current time or optionally any prior time selected by the user). Each line may contain data for one variable (e.g., a drug) or optionally, a set of related variables. A set of these lines is displayed on the screen or page (which may be the full set of all data for the patient or a subset chosen by the user). These lines may be synchronized, such that all have the same starting time, ending time, and time scale. This allows the user to see correlations and other relationships across data. In prior systems, these correlations and relationships were difficult or impossible to understand as they come from data that are provided by different sources and which is stored separately.

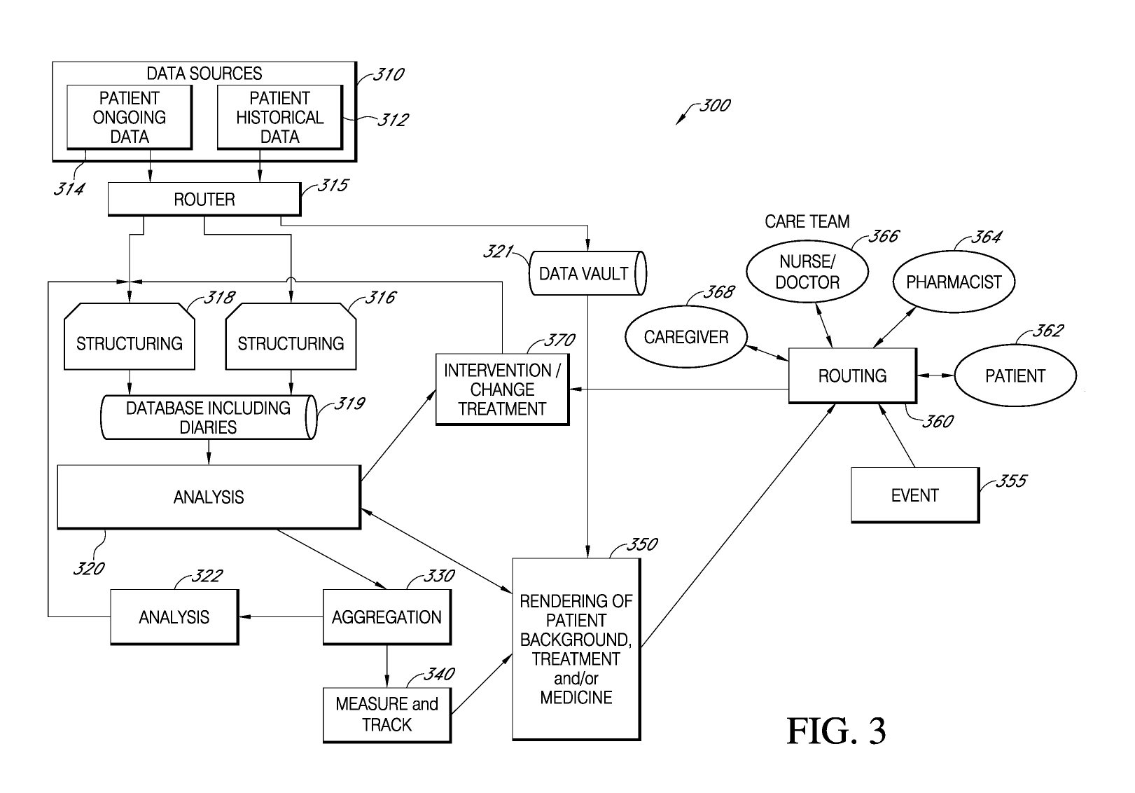

Referring now to FIG. 3, an example of an embodiment a process 300 of processing medical information for a particular owner or patient will be described. Data sources 310, such as described above, provide medical information such as patient background/historical information 312 and ongoing data such as treatment and/or medication information 314 along with any other previously mentioned medical information for a particular patient. In certain embodiments, the information 312 and information 314 is routed by router 315 to one or more of a structuring process 316, a structuring process 318 and a data vault 321. The data vault can store various patient information including scanned records, notes, etc. and image, audio and video data, and so forth in its captured format, for example. The router will be further described in conjunction with FIG. 4 hereinbelow. In one embodiment, the patient background information is structured by the structuring process 316 and ongoing patient data including the treatment and/or medication information is structured by the structuring process 318. In other embodiments, other arrangements may be used for structuring certain medical information. After the medical information is structured, which may include normalizing the information into a consistent and standardized record format, the information is stored in a diary for the particular patient in a database 319.

Information from a patient's diary can be accessed by an analysis process 320, which performs analysis by processing the complex relationships between data. This analysis of the patient background, treatment and/or medication information and other patient data can be rendered on the screen visually at a process 350 including but not limited to the form of color, highlighter, arrows, or indicators. The analysis can also be used by the system and method to suggest that a healthcare professional make changes to a drug or treatment, or to a clinical trial or experiment. If desired, the analysis process 320 can also be used to determine if there is a risk or critical event, or if a suggestion for medication and/or treatment or referral to a third party is appropriate. Means of determination include heuristics or algorithms that consider the patient's data. The output of this process can include an alert to be sent as a message. In certain embodiments, analysis process 320 can include trend detection, such as determining how long it will take an owner (patient) to reach certain goals, identifying harmful trends and so forth.

Analysis of the patient's data can include a recurrence system. The recurrence system uses a database pattern, which involves recognizing a set of table columns as representing recurrence data, e.g., did this event occur:
once at a specified moment in time?
repeatedly, at several regularly-occurring moments in time, starting at a specified time?
constantly, over a period of time, starting at a specified time?
and, when the event occurred regularly or for an extended period of time, when did that period end, or is it still ongoing?

The recurrence system is part of the database schema for the core database 163 and the application server code.

The output of the analysis process 320 can be sent to the rendering process and/or to an intervention or change treatment state 370. Some embodiments may determine the appropriate people to receive each message based on their expertise, relationship to the patient, authentication and other relevant information at a routing process 360 which receives input from the rendering process 350, or event information 355. The routing process can ensure that messages are sent to all appropriate recipients but not to anyone else. The potential recipient include, but are not limited to, a patient 362, a pharmacist 364, a nurse and/or doctor 366, and a caregiver 368, which are all part of the multi-disciplinary care team. The routing process 360 can also receive information from any member of the multi-disciplinary care team and route the information to the intervention or change treatment state 370. Any intervention or changed intervention information can be sent as a new data source to structuring process 318, for example.

The output of the analysis process 320 can also be sent to an aggregation process 330. The aggregation process 330 can include sorting data from disparate sources by time stamp, or other field including but not limited to age, gender, location, job, lifestyle choices, life events, underlying health conditions including pre-existing conditions, genetic identifier, symptoms, treatments, drugs, or healthcare provider. The aggregating may be expanded to include an optional step of aggregating data corresponding to a plurality of patients whose care is provided by a particular health care professional, health care facility, region, nation, and/or any particular form of treatment provision across a population or individually targeted subsets of a population. The output of the aggregation process 330 is sent to an analysis process 322, which can enable the user to correlate medications and treatments prescribed and performed to determine a number of factors regarding these care providers, including over- or under-prescription of medication, treatment effectiveness, superior or inferior diagnosing of particular symptoms or diseases, recognition of contraindications, and so forth. This may be useful to determine a healthcare provider's particular areas of expertise, and/or the effectiveness of a particular treatment regimen, and/or areas where additional training or education is needed. Results of the analysis can be stored in the LHD database 319 through the structuring process 318, for example.

The output of the aggregation process 330 is also sent to a measure and track process 340 to track, monitor and measure outcomes for medications or treatments as prescribed by a particular health care professional, health care facility, region, nation, and/or any particular form of treatment provision across a population, individually targeted subsets of a population, or a particular patient. The output of the measure and track process 340 can be used as an input to the rendering process 350, described above.

The system and method captures multiple types of data from different types of fields, captured from various different sources. The system and method repurposes any kind of medical data from any kind of source to be on graphical summary pages so as to be more useful and meaningful for the care team, including patient and family caregivers.

The following data fields in Table 1 below show several examples of data capture source. These are just illustrative; the data source in the right column could be any combination of the various data sources listed. An additional source that could be used for any of the data fields is an electronic medical record (EMR). The data extract from a data feed from an EMR, Pharmacy Management Software (PMS) or Lab Feed can be HL7-compliant XML data. The system and method effectively standardizes all the disparate data into a standard, consistent, easy-to-understand single format for all care team members to share and gain insight from.

TABLE 1

| Data Field | Captured By |
|---|---|
| Vital signs | Robot |
| Medications dispensed | Nurse or Pharmacist |
| Medications taken | Caregiver, Patient, Family |
| Test and Labs | Nurse or Physician |
| Immunizations | Nurse, Physician, or Lab Web Services |
| Signs | Nurse, Physician, or PMS Web Services |
| Symptoms | Caregiver, Patient, Family |
| Life events/Lifestyle | Caregiver, Patient, Family |

Referring now to FIG. 4, example structures and configurations of the patient historical data 312, patient ongoing data 314, router 315, and structuring 316 and 318 shown in FIG. 3 will be further described. In certain embodiments, the patient historical data 312 includes paper-based records 410 and electronic records 412. The paper-based records 410 can be processed by manual data entry 420 and electronic records 412 can be processed by automated import operation 422. In addition, the paper-based records 410 and the electronic records 412 can be routed by router 315 to a processing operation 430, such as an import process 432 which can be scanning of the paper-based records 410, for example. Other information such as electronic records 412 (e.g., a digital photograph) can be imported with minimal processing. The output of the processing 432 is stored in the data vault 321. Storing information in the data vault will be further described hereinbelow.

In certain embodiments, the patient ongoing data 314 includes manual data sources 414 and electronic data sources 416, such as health or monitoring devices. The manual data sources 414 can be processed by manual data entry 424 and the electronic data sources 416 can be processed by automated import operation 426.

In certain embodiments, the output of the manual data entry 420 is routed to a manual import process 440 of the structuring process 316. The output of the automated import operation 422 can be routed to an electronic records transformation process 442 of the structuring process 316. The output of the manual data entry 424 can be routed to a diary manual data input process 444 of the structuring process 318. The output of the automated import operation 426 can be routed to an electronic data stream transformation process 446 of the structuring process 318. After the information is structured by structuring process 316 and 318 to a common format, the information is stored in the diary database 319 and can be analyzed at the analysis process 320.

In further description, data documents can be imported to the diary. In certain embodiments, the diary includes many categories, types or modules of health or medical information. These can be either historical data (often bulk data from hospitals, general practitioners (GPs), or other repositories) or ongoing data, usually specific to a single action (e.g., filling a prescription at a pharmacy). They may be: 1) diary data, e.g., medical-prescriptions, which is used to create an entry for its corresponding diary module and 2) non-diary data, e.g., an X-ray image, which contains no information specific to a diary module, and appears only in the data vault. In other embodiments, the categories, types or modules of information can be for non-medical data.

In certain embodiments, every data document incoming to the diary is stored in the owner's data vault. Any document may contain one or more pieces of diary data. For instance, records from a GP may contain multiple conditions, symptoms, and treatments. In certain embodiments, a partner processes and then sends parsed medical data to the diary in an agreed format, along with the scanned document(s) in PDF format. Also included with the parsed data is an index for each data item, indicating the data's source in the accompanying PDF(s), indicating which document contains it, and the relevant page in that document.

The users of the system 100 can include 1) an owner of data (e.g., health data) who has an instance of a diary, 2) a visitor who has no diary but can be given selective access to an owner's diary (e.g., for a parent, sibling, friend, etc.), 3) a staff member who belongs to an organization (e.g., a doctor, nurse, technician, or administrative staff), and 4) an owner/visitor who has a diary but has also been given access to another owner's diary. A visitor can be further classified as a guest who can have read and/or write privileges or as a guardian who can use a diary with full owner privileges (e.g., for adults who have demonstrable legal authority to look after a child's or an incapacitated person's diary as if they were the owner).

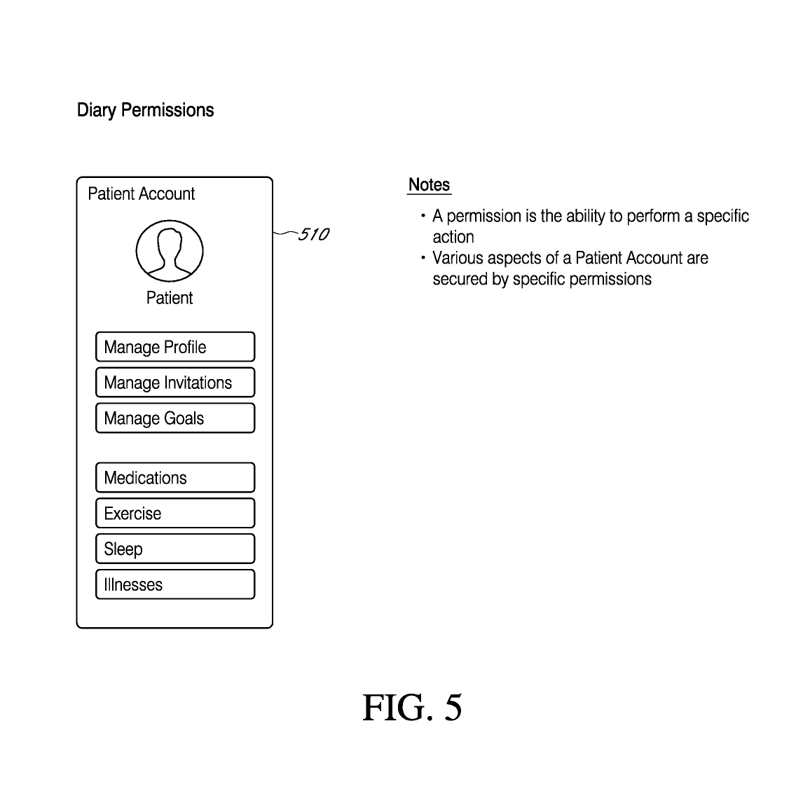

Referring to FIG. 5, example diary permissions will be described. A permission can be considered as an ability to perform a specific action. An example patient account 510 for a particular patient is shown as having various aspects including manage profile, manage invitations and manage goods along with categories, types or modules of information for medications, exercise, sleep and illnesses. These various aspects are secured by specific permissions.

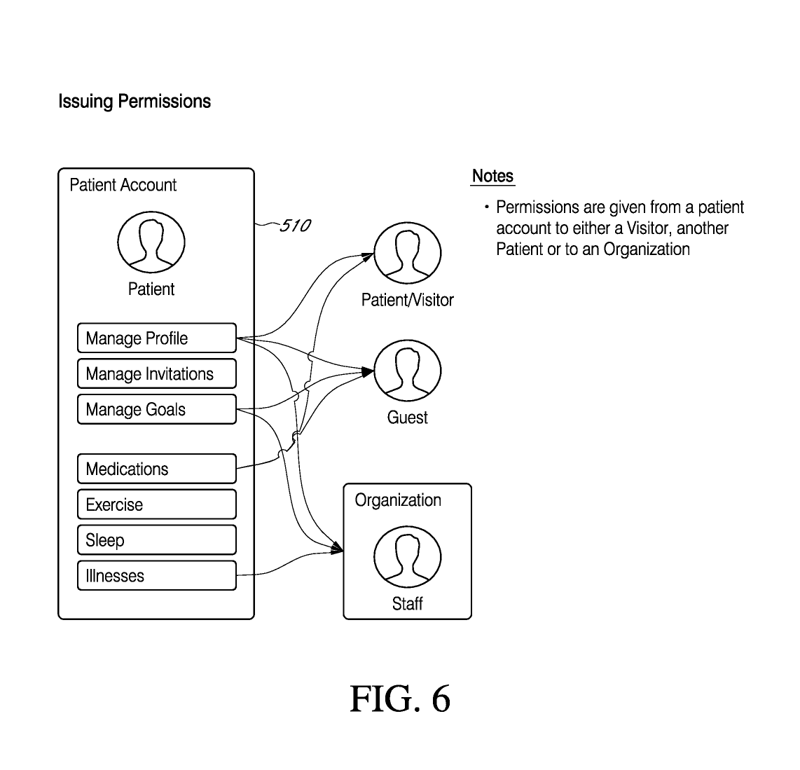

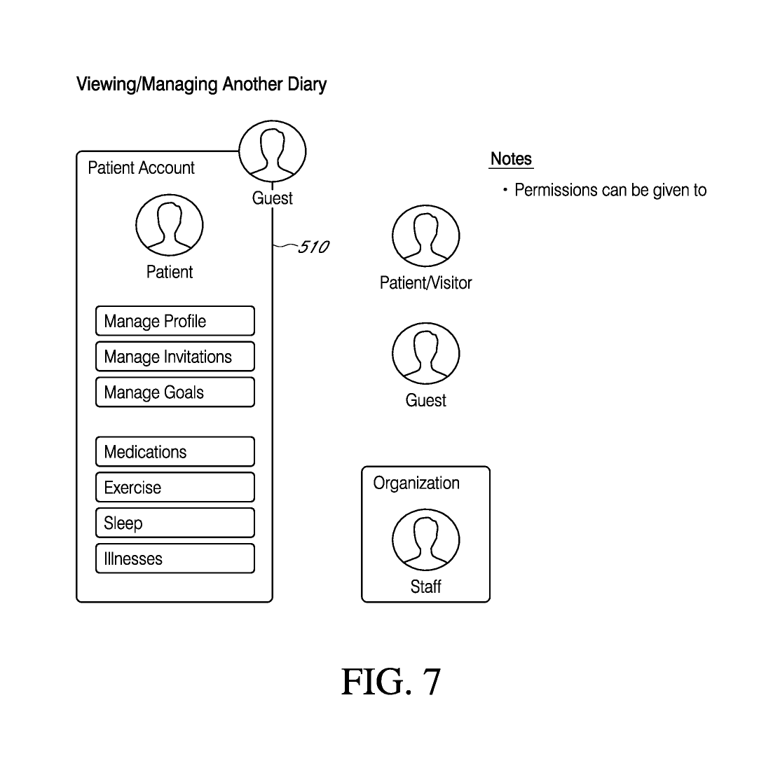

Referring to FIGS. 6 and 7, examples of issuing permissions will be described. Permissions are given from the example patient account 510 to one or more of another patient/owner, a visitor, a guest and a staff member of an organization. The patient/visitor is a third party patient/owner who has permission to view/update an owner's records in the diary.

A single permission controls access to a type of information held by the diary for an owner, or a feature pertaining to an owner or organization. Examples of information that is controlled by a permission are: exercise records, prescriptions and treatments. Examples of features that are controlled by a permission are: user profile and sending invitations to other users.

A permission can be private, read-only (meaning that adding, updating, or deleting information is not allowed) or can give full access (adding, updating, or deleting information is allowed).

In certain embodiments, permissions are applied in sets. A set of permissions can contain any combination of permissions, each of which can be individually read only, full or private. These sets can be given by an owner to third parties via an invitation.

Permissions are hierarchical, such as for example (omitting the concept of read-only): 1) permission to an owner's entire account; 2) permission to all of an owner's diary data; and 3) permission to exercise data.

In this case, if any user of the LHD application attempts to view exercise data for a particular owner, and does not have any of the permissions in the above list, this action would be prevented. The user must have any one of these permissions in order to access exercise data. If the user has only permission 3 and attempts to access any piece of diary data other than exercise data, access would be denied. If the user has permission 2 or 1, this would encapsulate all diary data permissions, so access to all diary data would be allowed.

‘Read-only’ is a concept that can be applied to any permission. In the list above, a user could be given a read-only permission of type 1, 2, or 3. If the user has read-only access of type 2, ‘permission to all of an owner's diary data’, the user can access all diary data, but can make no changes to it. In addition, this user could have a full, non-read-only permission of type 3, ‘permission to exercise data’, in which case they would be able to make changes to exercise data, but all other diary data would remain read-only for that user.

An Owner can give permissions to their data to another individual (termed a guest) via an invitation. Once the invitation process is complete, the guest is able to view selected data from the owner's diary. The guest can see or update a specific piece of data only if the diary's owner has given the guest a corresponding permission.

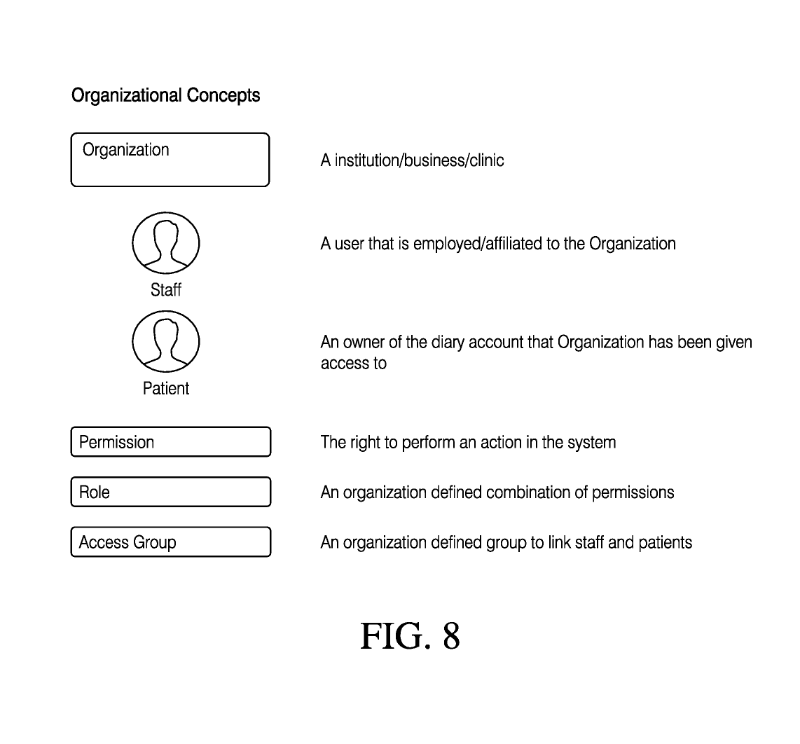

Referring to FIG. 8, examples of organizational concepts will be described. An organization can be an institution, business or clinic, for example. The staff person can be a user that is employed by or affiliated to the organization. The patient/owner is an owner of the diary account that the organization has been given access to. A permission can be the right to perform an action in the system. A role can be an organization-defined combination of permissions. An access group can be an organization-defined group to link staff and patients.

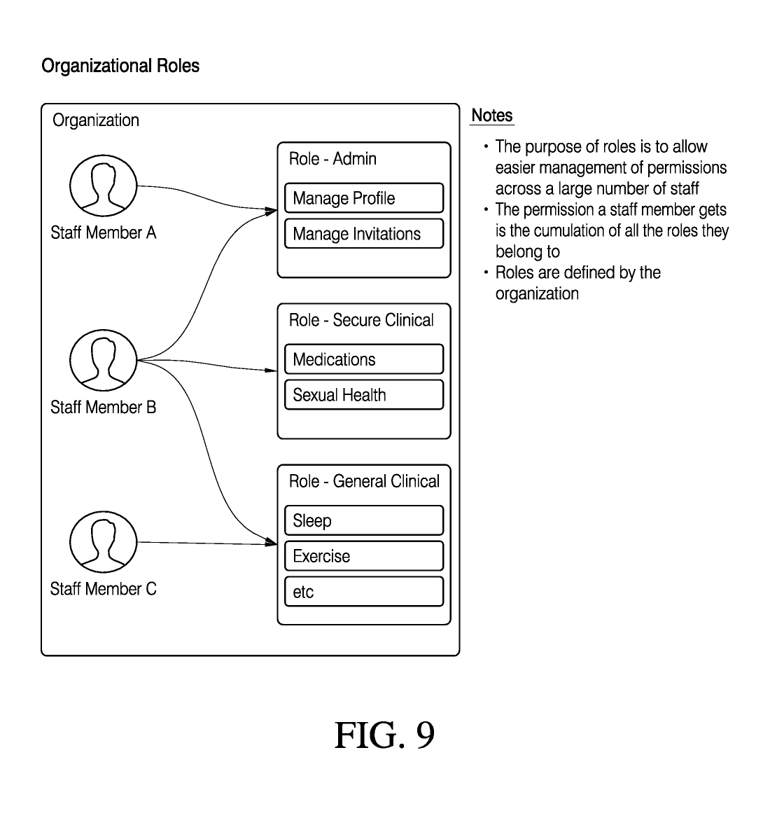

Referring to FIG. 9, examples of organizational roles will be described. In certain embodiments, a purpose of roles is to allow easier management of permissions across a large number of staff members. The permission a staff member obtains is the accumulation of all the roles they belong to. Roles can be defined by each organization. The example shown in FIG. 9 illustrates an administration role, a secure clinical role and a general clinical role with staff members A and B being assigned to the administration role, staff member B being assigned to the secure clinical role, and staff members B and C assigned to the general clinical role.

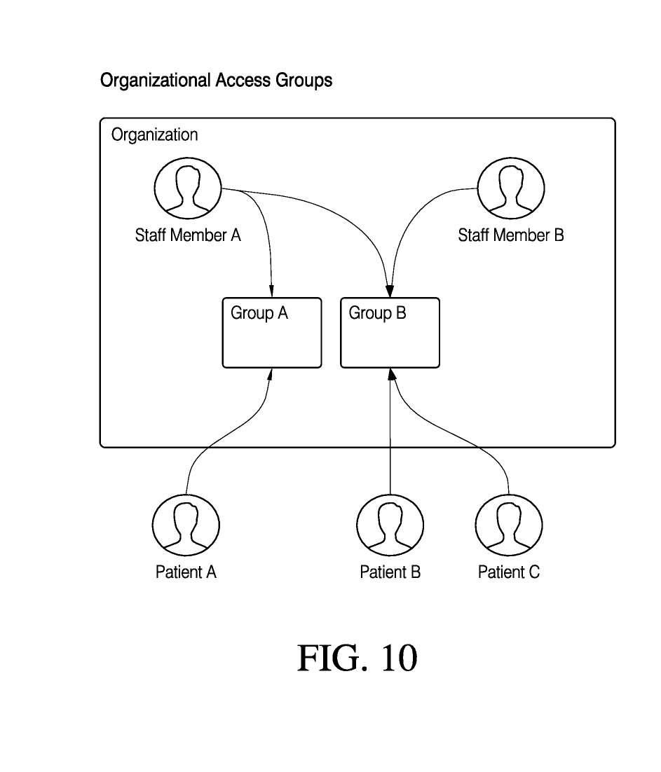

Referring to FIG. 10, an example of organizational access groups will be described. The example shown in FIG. 10 illustrates a group A and a group B, where staff member A is assigned to both group A and B, and staff member B is only assigned to group B, and where patient A is assigned to group A and patients B and C are both assigned to group B.

Thus, permissions can be given by an owner to an organization, similarly to the way an owner would give them to a guest. An organization can create access groups. The organization can then assign its owners or patients to the access groups as it sees fit. An owner may be a member of more than one group at once. Similarly, the organization can assign its staff members to an access group, representing the staff members who will be working with the members of that group. A staff member may belong to more than one group in the same organization.

Organizations can create roles, which are groups of their own staff members. These roles can then be allocated permissions by the organization. Then, staff members in that group may only see or update data from an owner's diary if: 1) the owner has given the organization a corresponding permission, and 2) the organization has also given the staff member's role the same (or more elevated) permission, and 3) the owner is in an access group to which the staff member belongs.

Roles are also used to control access to the organization's administrative functions. There is a distinction between the administrative and health/medical roles.

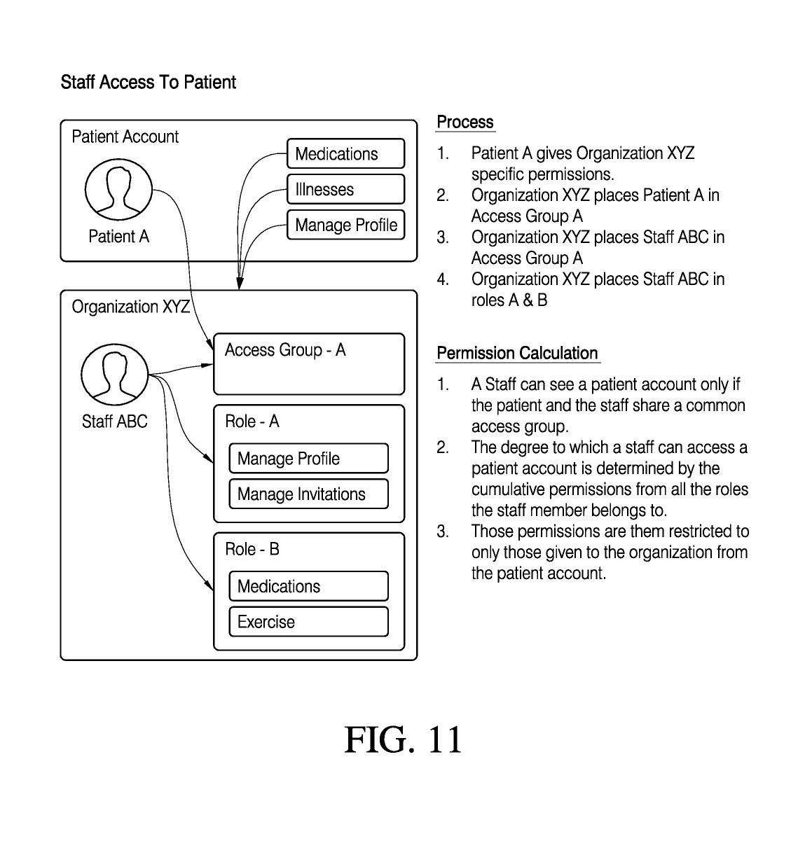

Referring to FIG. 11, an example of staff member access to an owner will be described, including access group/staff role concepts. An organization can place an owner in one or more access groups, and can place staff in one or more roles. Owners can give permissions to organizations, and organizations can specify which permissions are devolved to each role. In this way, the patient (referred to in the LHD ecosystem as an owner) has full control over access to their records at all times. The organization is able to specify the level of access given to its staff members, and which staff members have access to each access group.

In the example of FIG. 11, owner A gives organization XYZ specific permissions. Organization XYZ place owner A in access group A. Organization XYZ places staff member ABC in access group A. Organization XYZ place staff member ABC in roles A and B. A staff member can view an owner's account only if the owner and the staff member share a common access group. The degree to which a staff member can access an owner's account is determined by the cumulative permissions from all the roles the staff member belongs to. Those permissions are then restricted to only those given to the organization from the owner's account.

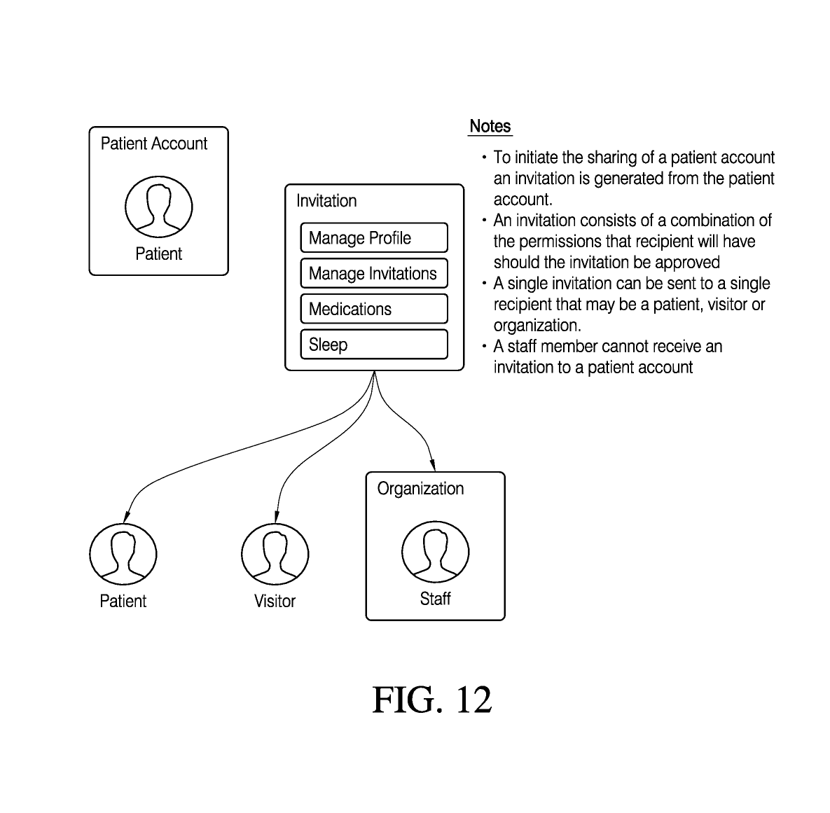

Referring to FIG. 12, an example of an invitation is illustrated. An owner is able to invite a guest (third party) to access the owner's diary records. This process is managed within the diary application. The process varies depending on whether the invitee is a current user or not.

To initiate the sharing of an owner's account an invitation is generated from the owner's account. An invitation consists of a combination of the permissions that recipient will have should the invitation be accepted. A single invitation can be sent to a single recipient that may be a patient, visitor or organization. A staff member cannot directly receive an invitation to an owner's account.

### Data Vault and Module Data Creation

Module data and data vault contents can be generated in several ways, including:
1) from paper documents, via a scanning and data-entry process performed by a diary's owner, or LHD and/or its designated partners;
2) from third-party data repositories, either in bulk as a history import, or on an ongoing basis to update the diary with live data;
3) from devices such as vital signs monitors, electronic scales, exercise monitors, by:
   a) integration with the device via LHD's mobile or desktop application(s);
   b) integration between the device vendor's back-end systems and those of the diary;
   c) any other intermediary;
4) by direct data entry by the owner or their representative of data into one of the diary's user interfaces.

Any incoming data may incorporate data to be stored in the data vault, and/or diary module data. Not every piece of data will include both. Where both are included, a user of the diary can navigate directly to the associated data in the data vault from an item of diary data being viewed. Where possible, this link can be created automatically at the same time the diary and data vault content are created.

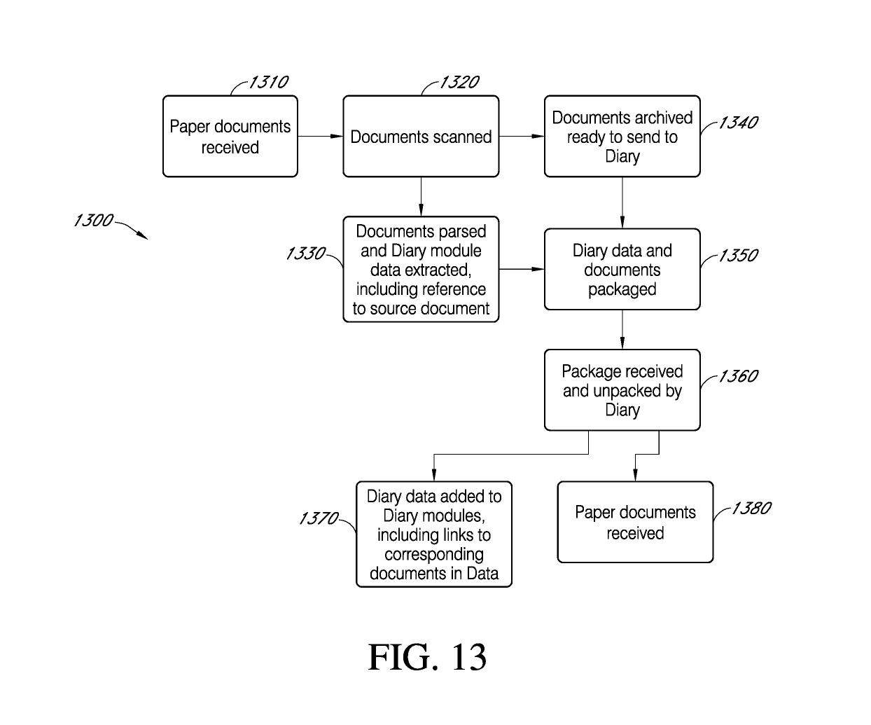

FIG. 13 illustrates an overall process 1300 used to import historical paper documents into the diary. A similar process is used for importing any data from documents (history, updates, paper, and electronic). It is also possible for a user of the diary to establish a link between pre-existing module data and documents in the data vault. The module data and data vault document may have been created by a user, or by some automated process; this process does not depend on the original source of these items.

Beginning at state 1310, source paper documents are received and then scanned at state 1320 by any of well-known scanners. Moving to a state 1330, the scanned documents are parsed and data for one or more of the diary modules is extracted including a reference to the corresponding source documents.

After the documents are scanned at state 1320, the process 1300 archives the documents at state 1340 so as to be ready to be sent to the diary. In certain embodiments, the scanned documents are collated into bulk PDFs, such as with a limited number of pages, or a maximum file size. For example, if 400 pages come in at state 1320, they could be collated into four PDFs of 100 pages each in state 1340. After the completion of states 1330 and 1340, process 1300 advances to state 1350 where the PDFs are made available so that a single chunk of data incorporating the parsed diary data (text/numerical) and the PDFs can be sent to the diary system for inclusion in the owner's diary. At state 1350 the diary data and the documents are packaged. Proceeding to state 1360, the package is received and unpacked by the diary. The diary data is added to the diary modules at state 1370, including links to corresponding documents in the data vault. In addition, the documents are added to the data vault at state 1380.

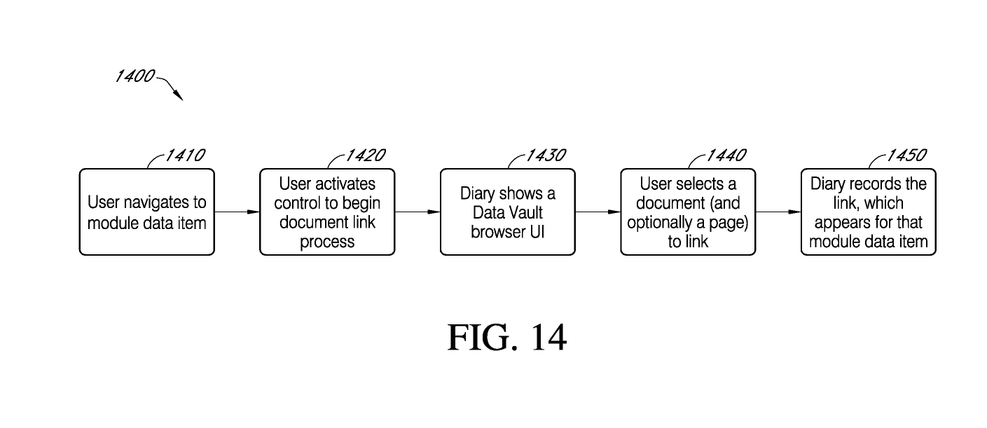

FIG. 14 is a flowchart illustrating an embodiment of a process 1400 for linking documents. In certain embodiments, the process can be performed on the system 100 illustrated in FIG. 2A. Beginning at a state 1410, a user navigates to a module data item. At a state 1420, the user activates a control to begin a document link process. At a state 1430, a data vault browser user interface is shown to the user. At state 1440, the user selects a document (and optionally a page) to link. At state 1450 the diary records the link which appears for that module data item.

Once a link exists between an item of module data and a data vault document, the link appears wherever the details of that module's data items are viewed. When clicked, the document opens (to the correct page where a page index is included and can be viewed), or if the document is not viewable within the application's environment (web browser, mobile app, etc.), the user is prompted to download/view the document externally in a normal way. This can be achieved by implementing the link in a standards-compliant manner, e.g., for PDFs: https://www.1hdservencom/DataVault/medicaldocument.pdf#page=50. Security is maintained with access to documents mediated by the permissions system.

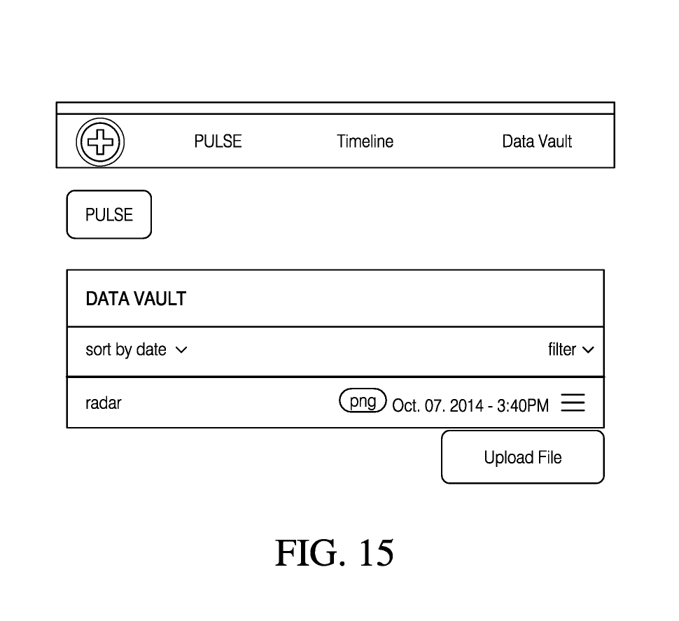

FIG. 15 is an example of a screen display illustrating accessing a data vault. In certain embodiments, FIG. 15 illustrates an embodiment of a main data vault page. The page shows options to sort by date and filter. This is done purely via normal interactions between the User Interface/API and the database. No reference to the backing store is required.

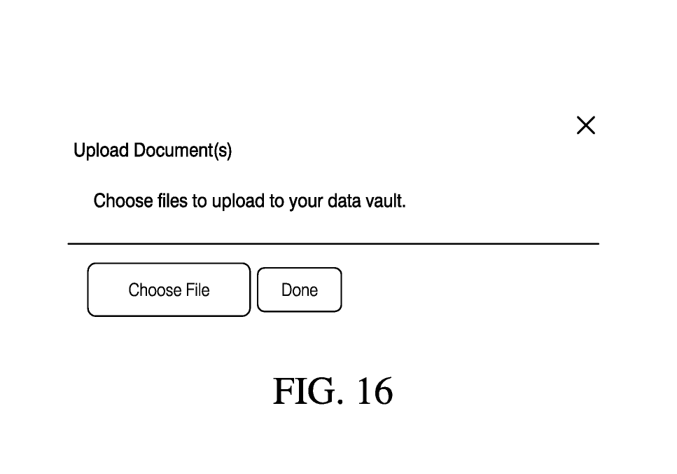

FIG. 16 is an example of a screen display illustrating an upload screen for the data vault. On clicking the upload file button in FIG. 15, the user sees an upload dialog as shown in FIG. 16. The user (e.g., owner) can select the choose file button to choose files to upload to their data vault. For local/disk/flat data vault files, an upload is performed directly to the web application's HTTP server, and the file is written to disk, either local to the server, or as a network share. For a remote data vault, the following is performed:
The client (web or API) notifies the application that it's ready to upload a data vault document.
The application generates a URL for the calling client to use to upload the document. This will usually be created by tools provided by a bulk data storage provider, e.g., Amazon AWS S3. It may be a local URL, however, if the application will proxy the upload itself, or if the file is to be stored locally.
The application responds to the client with the URL generated above, plus other metadata it may need (headers, HTTP action, etc.).
The client performs an HTTP upload (generally a POST) to the specified URL.
The client calls the application to inform it that the upload has completed successfully. The document is now available for use in the data vault.

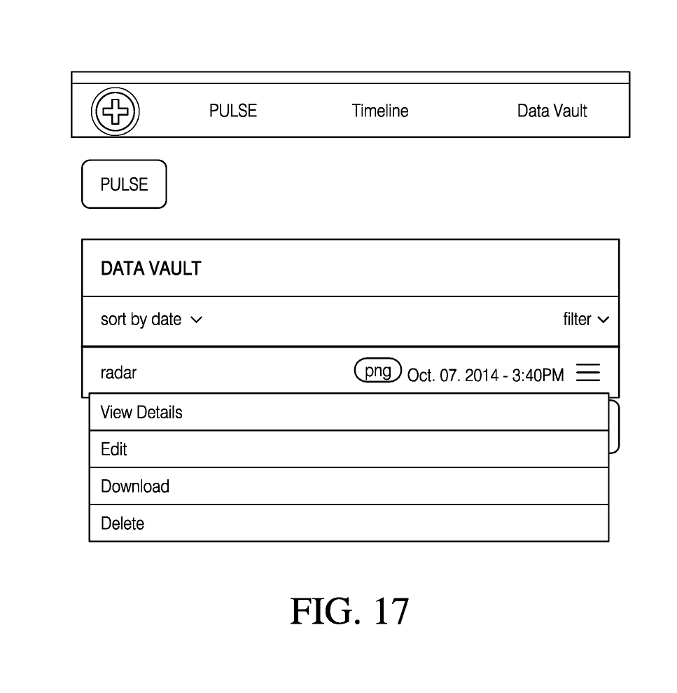

FIG. 17 is an example of a screen display illustrating user options for the data vault. The user can click the context menu control (the three bars icon at the right of the file's entry) to see the context menu for the chosen file. The “view details” option causes a read-only view of the file's details. Furthermore the user can edit, download or delete the file as applicable and allowable.

Retrieving a Data Vault document includes one of two processes as follows:
The client tries to download the document from the application server. This will then serve the file, or respond with a redirect to the third-party bulk data storage facility, as required.
The client requests a URL from which to download the document. The application then sends either a local URL, or a URL that refers to the third-party bulk data storage facility, as required.

In order to support older API clients, the old direct upload/download API functions can be still supported. If they're called, the application performs the steps above on behalf of the client, effectively proxying the upload or download.

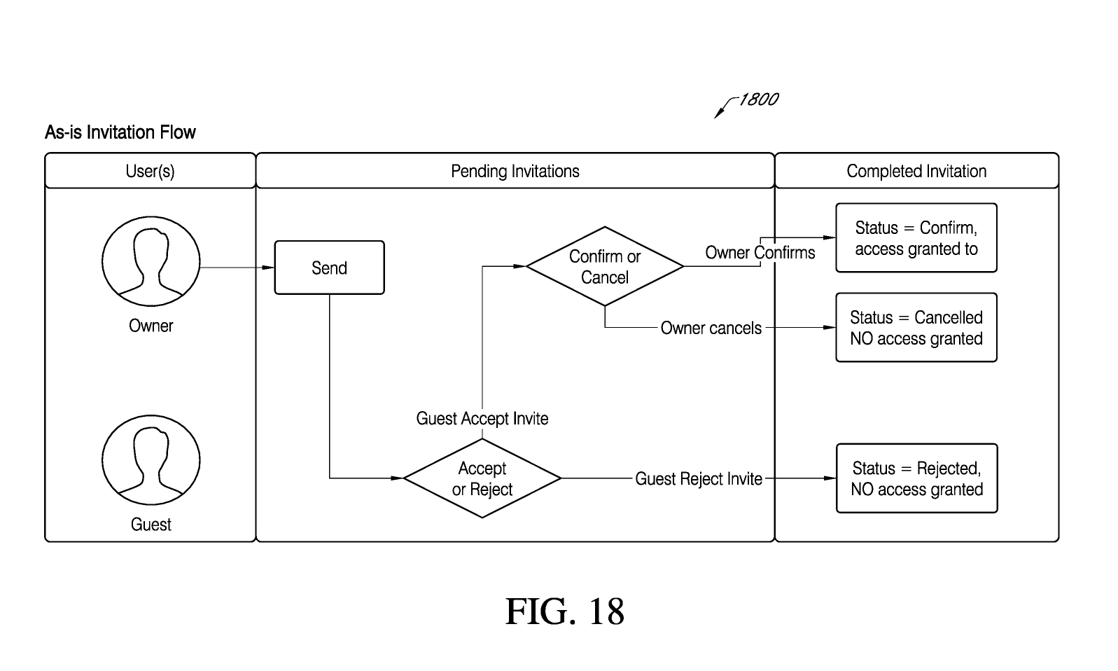

FIG. 18 is a diagram illustrating an invitation flow 1800 between an owner and a guest. In certain embodiments, the process can be performed on the system 100 illustrated in FIG. 2A. The steps of the invitation flow are as follows:

1. Owner issues invitation
Notification sent to Guest—*Note: email does NOT include link
Invitation listed in owners account—Pending, status=“Sent”
Invitation listed in guest account—Pending, status=“Sent”
   2 Guest Accepts or Rejects invitation

a. Accepts—
Invitation listed in guest account—ation/Pending, status=“Accepted”
Invitation listed in owner account—Pending, status=“Accepted”

b. Rejects—
Invitation listed in guest account—/Invitation/Completed, status=“Rejected”
Invitation list in owner account—/Invitation/Completed, status=“Rejected
Invitation workflow complete—NO access granted
NO notification is sent to owner informing of invitation rejection
   3. Owner Confirms or cancels invitation

a. Confirms—
Invitation listed in owner account—/Invitation/Completed, status=“Confirmed”
Invitation listed in guest account—/Invitation/Completed, status=“Confirmed”
Invitation workflow complete—Guest can now access owner account

b. Cancels—
Invitation listed in owner account—/Invitation/Completed, status=“Cancelled”
Invitation listed in guest account—/Invitation/Completed, status=“Cancelled”
Invitation workflow complete—NO access granted
*NO notification is sent to guest informing of invitation cancellation

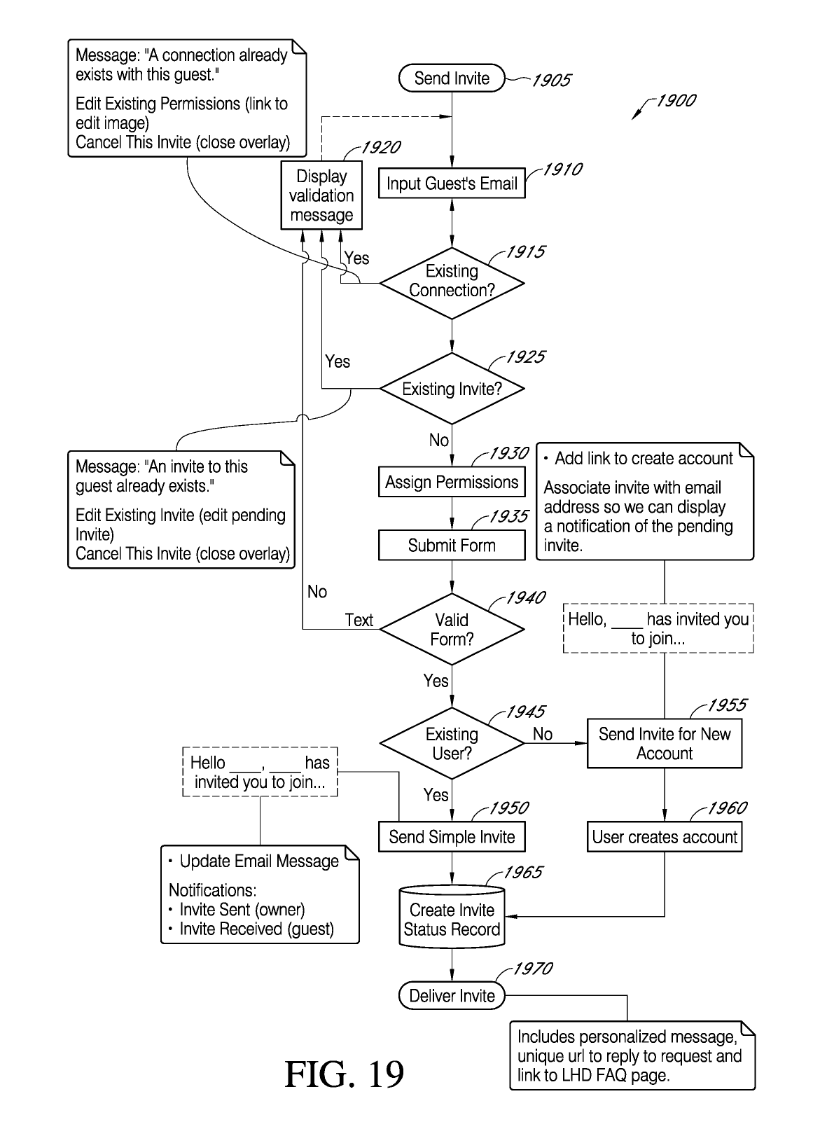

FIG. 19 is a flowchart illustrating an embodiment of a process 1900 for inviting a guest. In certain embodiments, the process can be performed on the system 100 illustrated in FIG. 2A.

Beginning at a state 1905, process 1900 advances to state 1910 to receive an input of an electronic address, such as an email address of a non-owner, e.g., a guest. Process 1900 moves to a decision state 1915 to determine if there is an existing connection with the guest having the electronic address entered at state 1910. In certain embodiments, this check is always made. It prevents a new invitation being sent to someone who already has a connection or invite; the user must edit those instead of sending a new invite. After that point, process 1900 checks for existing user controls as to whether the invitee is invited to create an account first, or their current account will be used to form the connection. If there is an existing connection as determined at decision state 1915, process 1900 moves to state 1920 and displays a validation message such as the phrase “a connection already exists with this guest” and options to edit existing permissions via a link to an edit page, or to cancel this invite which closes the overlay.

If there is no existing connection as determined at decision state 1915, process 1900 moves to a decision state 1925 to determine if there is an existing invitation. If there is an existing invitation, process 1900 moves to state 1920 and displays a validation message such as the phrase “an invite to this guest already exists” and options to edit the existing invite (to edit the pending invite), or to cancel this invite (which closes the overlay). After the completion of state 1920, process 1900 then continues at the state 1910 to input an electronic address for a guest.

If there is no existing invite as determined at decision state 1925, process 1900 proceeds to state 1930, the owner of the data assigns permissions to be granted to the guest. The assigning of permissions will be further described hereinbelow. Continuing at state 1935, a form with the invitation information is submitted such as when the user clicks the OK button on the dialog. This dialog has fields completed in states 1910 and 1930, identifying the invitee and the permissions to be given. At state 1935 process 1900 sends this data to the LHD web application server 150′, which can check the validity of the information (e.g., having a well-formed email address), and prompt the user to fix any problems found. Moving to a decision state 1940, process 1900 determines whether the form is valid. If the form is determined to not be valid at decision state 1940, a validation message is displayed at state 1920 and process 1900 then continues at the state 1910 to input the electronic address for a guest. If the form is valid as determined at decision state 1940, process 1900 advances to a decision state 1945 to determine if there is an existing user. If there is an existing user, process 1900 moves state 1950 to send a simple invitation including, for example, the phrase “Hello <user>, <owner> has invited you to join . . . ”. Along with the email message, updates are done by process 1900 which generates notifications of the invite sent (to owner) and invite received (for guest).

However, if there is no existing user as determined at the decision state 1945, process 1900 advances to state 1955 to send an invitation for a new account and prepares, for example. a phrase “Hello, <owner> has invited you to join . . . ” and adds a link to create an account. The invitation is associated with the email address so the system can display a notification of the pending invitation. Process 1900 then continues at state 1960 so the user can create an account. At the completion of either state 1950 or state 1960, process 1900 advances to state 1965 where an invite status record is created and stored in the core database 163 (FIG. 2B). Proceeding to state 1970, the invitation is delivered to the recipient, including a personalized message, a unique uniform resource locator (URL) to reply to the request and a link to a frequently asked questions (FAQ) page associated with the system 100.

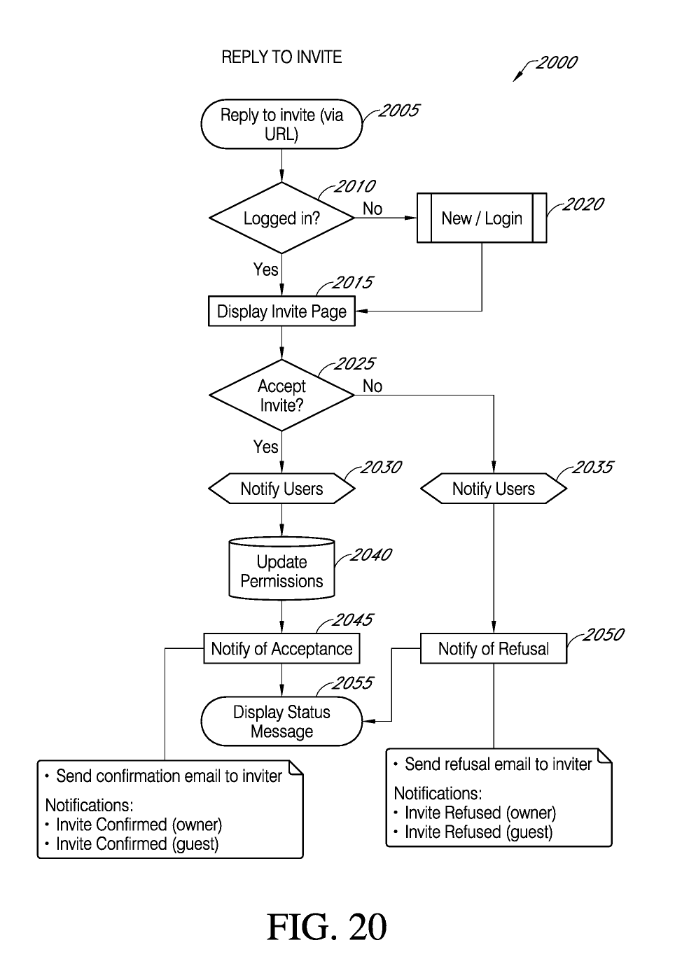

FIG. 20 is a flowchart illustrating an embodiment of a process 2000 for replying to an invitation via a URL. In process 2000, the user is a guest. In certain embodiments, the process can be performed on the system 100 illustrated in FIG. 2A. Beginning at a start state 2005, process 2000 advances to a decision state 2010 to determine if the user is logged in. If the user is logged in, process 2000 moves to a state 2015 to display an invite page. If the user is not logged in as determined at decision state 2010, process 2000 moves to state 2020 to request that the user login or begin a process for a new login. After completion of state 2020, the user is logged in and advances to state 2015 to display the invitation page.

Advancing to a decision state 2025, process 2000 determines whether the invitation is accepted. If the invitation is accepted, the process 2000 notifies the appropriate user of the system 100 at state 2030 of the accepted invitation. The user can be the inviter (if an owner sent an invitation to their own diary), or both an inviting guest and the owner (if a guest sent an invite to the diary of someone they are a caregiver for). However, if the invitation is not accepted at the decision state 2025, the process 2000 notifies the users of the system 100 at state 2035 of the declined invitation. At state 2050, process 2000 sends a refusal email to the inviter and generates notifications of invite refused (for owner) and invite refused (for guest), and moves to state 2055 to display a status message associated with the refusal of the invitation.

However, if the invitation is accepted and users are notified at state 2030, process 2000 proceeds to state 2040 and the permissions corresponding to the invitation are updated, such as in a permissions database. If the permissions are updated at state 2040, process 2000 advances to state 2055 to notify the appropriate user of acceptance of the invitation including sending a confirmation email to the inviter and notifications are generated that the invitation is confirmed for the owner and for the guest. Process 2000 displays a status message regarding the accepted invitation at state 2030 and the process 2000 ends.

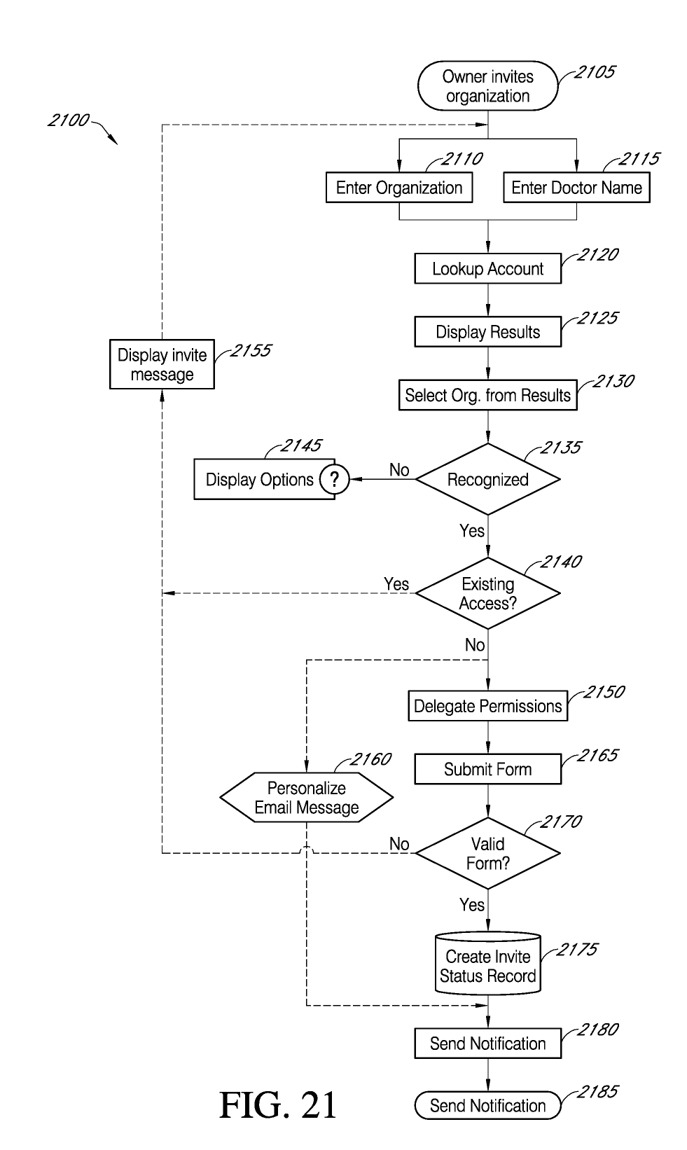

FIG. 21 is a flowchart illustrating an embodiment of a process 2100 for an owner of data to invite an organization. In certain embodiments, the process can be performed on the system 100 illustrated in FIG. 2A. Beginning at a start state 2105, the owner invites an organization by either entering the name of the organization at state 2110 or entering the name of a doctor at state 2115, where an organization to which the doctor can be determined at states 2120 to 2130, for example. At state 2120 process 2100 looks up the account of the organization. Proceeding to state 2125 results of the look-up for the organization are displayed by the process 2100, and a selection of a desired organization is received at state 2130. Advancing to a decision state 2135, process 2100 determines if the selected organization is recognized by the system 100. If the organization is not recognized, process 2100 moves to a state 2145 and displays possible options to the owner. If the organization is recognized as determined at the decision state 2135, process 2100 continues at a decision state 2140 to determine if the organization has existing access to the owner's account. If the organization has existing access, process 2100 moves to state 2155 and displays an inline message to the owner about the existing access. After the message is displayed, process 2100 returns to a screen where the name of an organization or doctor can be entered at states 2110 or 2115.

Continuing at the decision state 2140, if there is no existing access, process 2100 moves to state 2150 to delegate permissions to the organization. In parallel to the path to delegate permissions, state 2160 allows the owner to personalize the email message to the organization. After the delegation of permissions at state 2150, process 2100 advances to state 2165 where the form with the permissions is submitted and is checked for validity at a decision state 2170. If the form is not valid, process moves to state 2155 and displays a corresponding inline message to the owner. If the form is valid as determined at the decision state 2170, process 2100 proceeds to state 2175 where an invite status record is created, such as in an Invitation table of the core database 163. Proceeding to state 2180, the personalized email message from state 2160 is sent as part of the notification and the process 2100 ends with the sent notification.

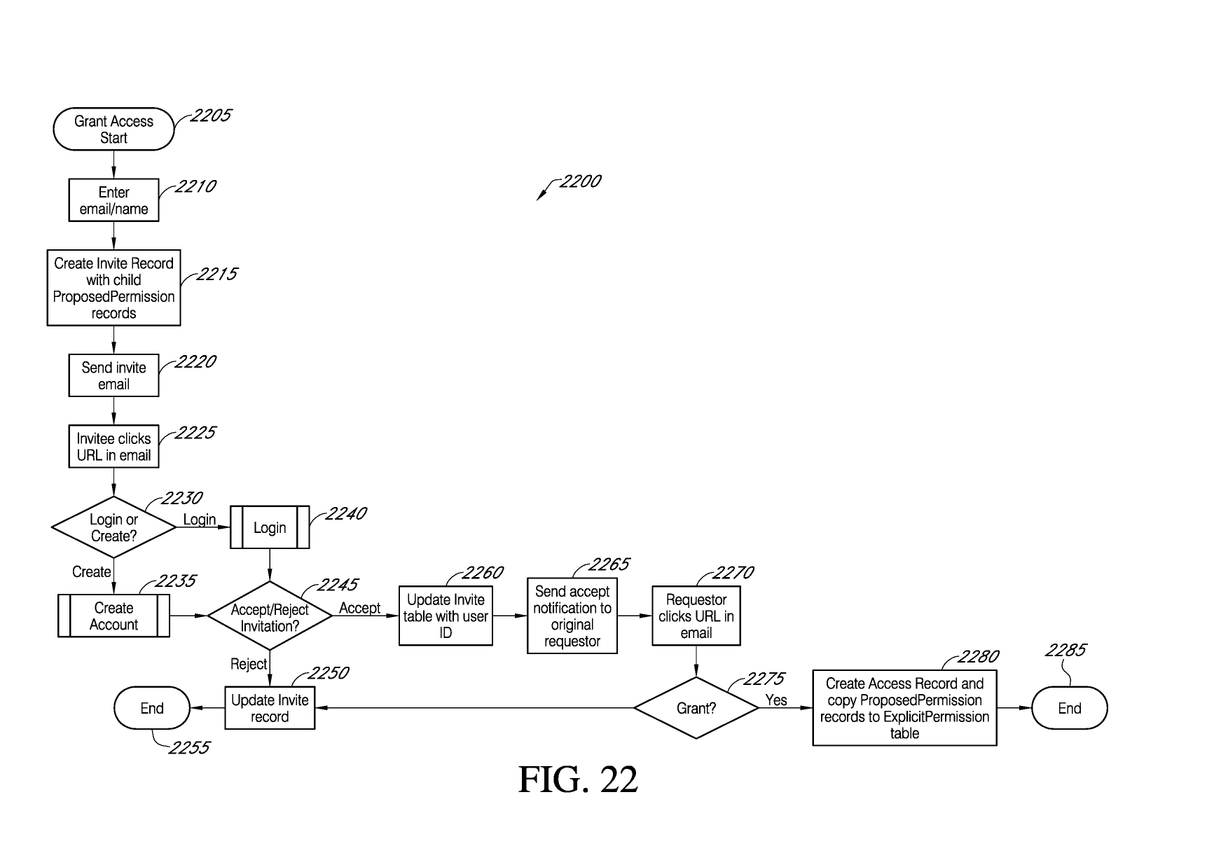

FIG. 22 is a flowchart illustrating an embodiment of a process 2200 for granting access to a diary. In certain embodiments, the process can be performed on the system 100 illustrated in FIG. 2A. Beginning at a state 2205, process 2200 moves to a state 2210 enters a name of a person or entity in an electronic communication, such as an email. Proceeding to state 2215, process 2200 creates an invitation record with child proposed permission records. Continuing at state 2220, process 2200 sends an invite email to the person or entity. Continuing at state 2225, the invitee clicks on a URL in the email and the process 2200 determines whether a login or a create account action is needed at the decision stale 2230. If the invitee does not have an account with the system 100, process 2200 moves to function 2235 where an account is created for the invitee and a login can be performed after the account is created. If the invitee has an account with the system 100 as determined at decision state 2230, process 2200 moves to function 2240 where the invitee can login to the system. At the completion of function 2235 or 2240, process 2200 determines if the invitation is accepted or rejected by the invitee. If the invitation is rejected, process 2200 continues at state 2250 where the invite status record is updated based on the rejection and the process ends at end state 2255.

However, if it is determined at decision state 2245 that the invitation is accepted, process 2200 advances to state 2260 and updates the Invitation table in the core database 163 with the user identification. Continuing at state 2265, process 2200 sends an acceptance notification to the original owner requestor via an electronic communication channel (e.g., email). Moving to state 2270, the owner requestor selects the URL corresponding to the invitee in the email and then as determined at a decision state 2275 either grants or confirms the acceptance by the invitee or cancels the invitation. If the invitation is canceled, process 2200 proceeds to state 2250 where the invitation record is updated to reflect the cancellation. If the invitation is granted as determined at decision state 2275, process 2200 advances to state 2280 and creates an access record and copies the proposed permission records to an Explicit Permission table in the core database 163. Process 2200 ends at an end state 2285.

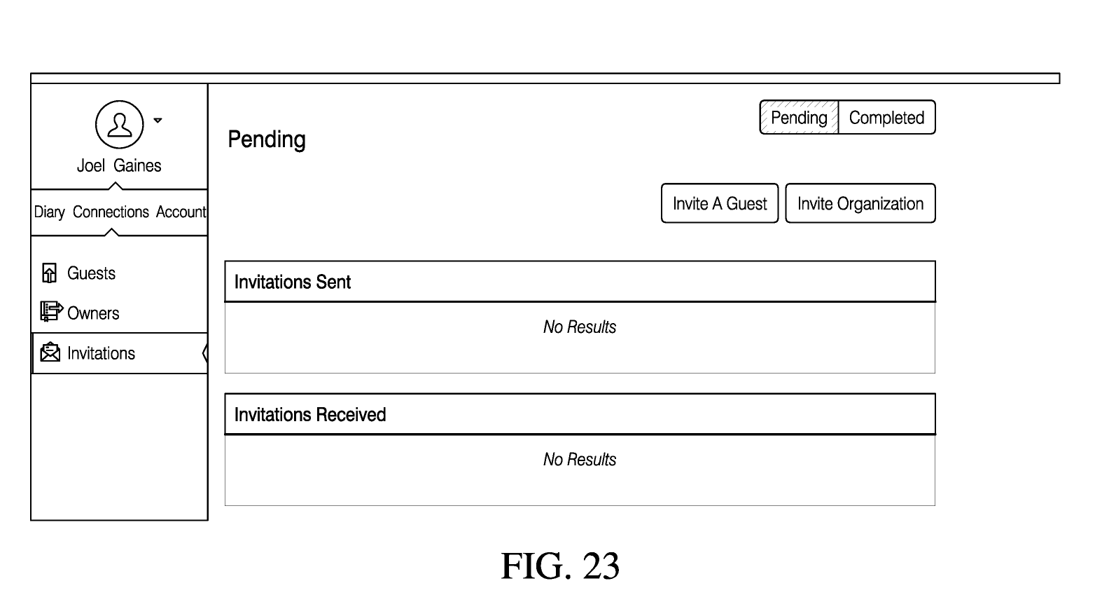

FIGS. 23-29 illustrate examples from the invitations user interface. FIG. 23 is an example of a screen display illustrating a screen for viewing current invitations and for new invitations to be made in the invitations process. An Owner can view their current invitations. On this page, they can also invite a new guest to view their diary, or invite an organization to participate in the owner's healthcare.

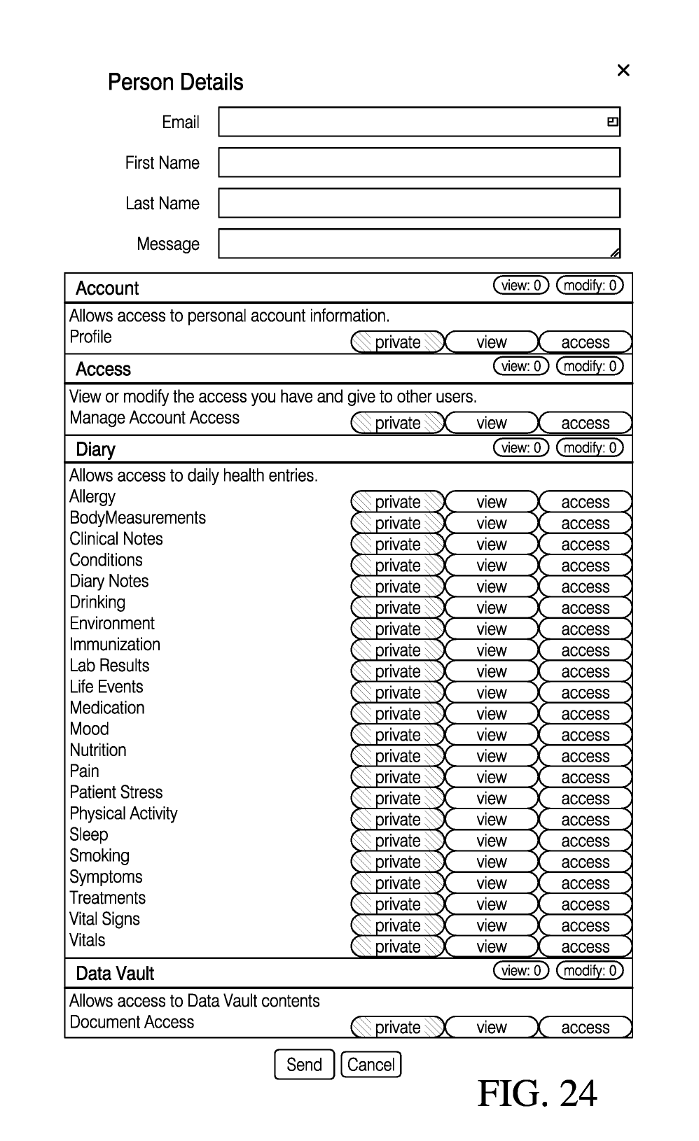

FIG. 24 is an example of a screen display illustrating an interface screen for entering information about a guest and permissions to grant for the guest. On clicking the ‘Invite a Guest’ button, a dialog is shown in which the owner can enter the details of the person to invite, and the permissions they would like to give that person. For example, the owner can select among private (none), view (read) and access (write) permissions from account, access, diary, and data vault group headings where, in certain embodiments, the diary has multiple categories/types/modules of data each having the option for private, view and access permissions.

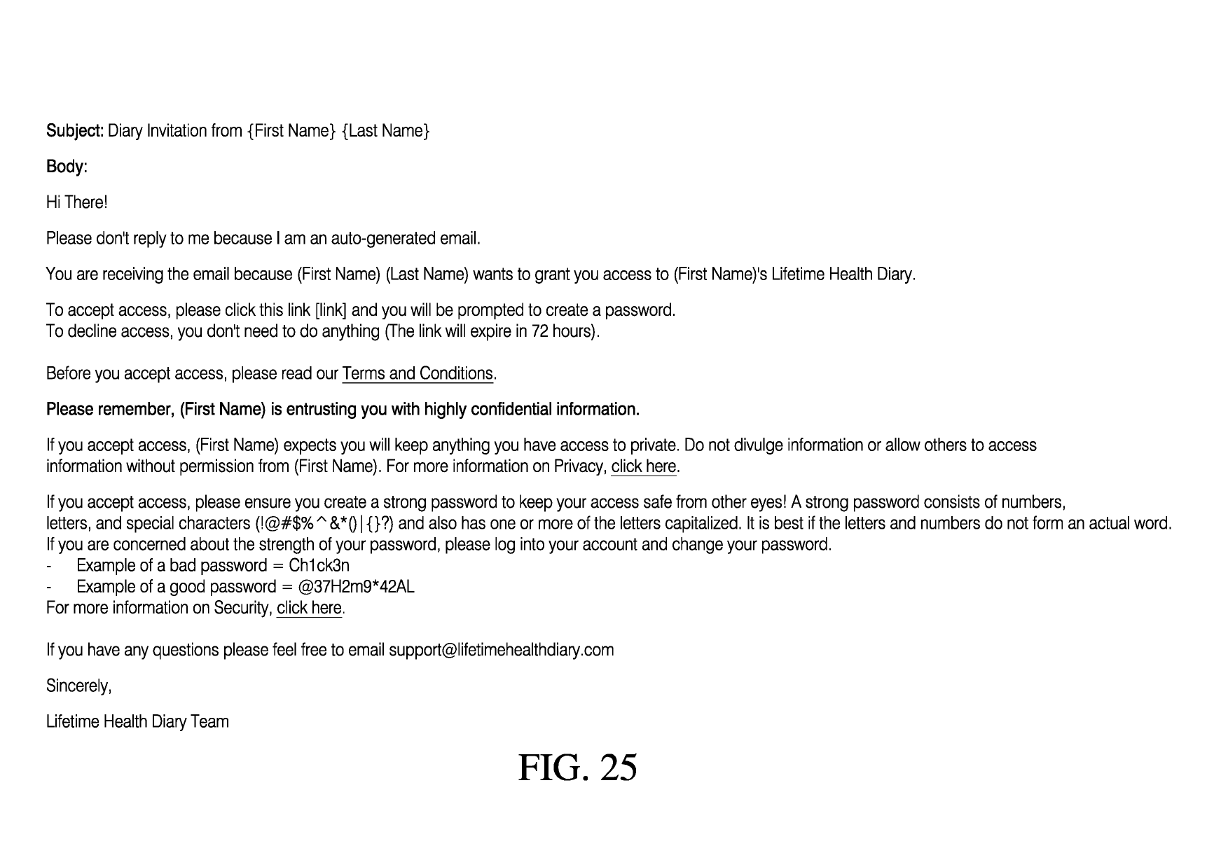

FIG. 25 is an example of a screen display illustrating an example emailed invitation. Once the details are complete and the permissions are chosen, the send button can be clicked in the display screen of FIG. 24, thus sending an invitation email to the invitee, such as the example email shown in FIG. 25. The invitee can be asked to create a strong password and to read the terms and conditions for using the system 100.

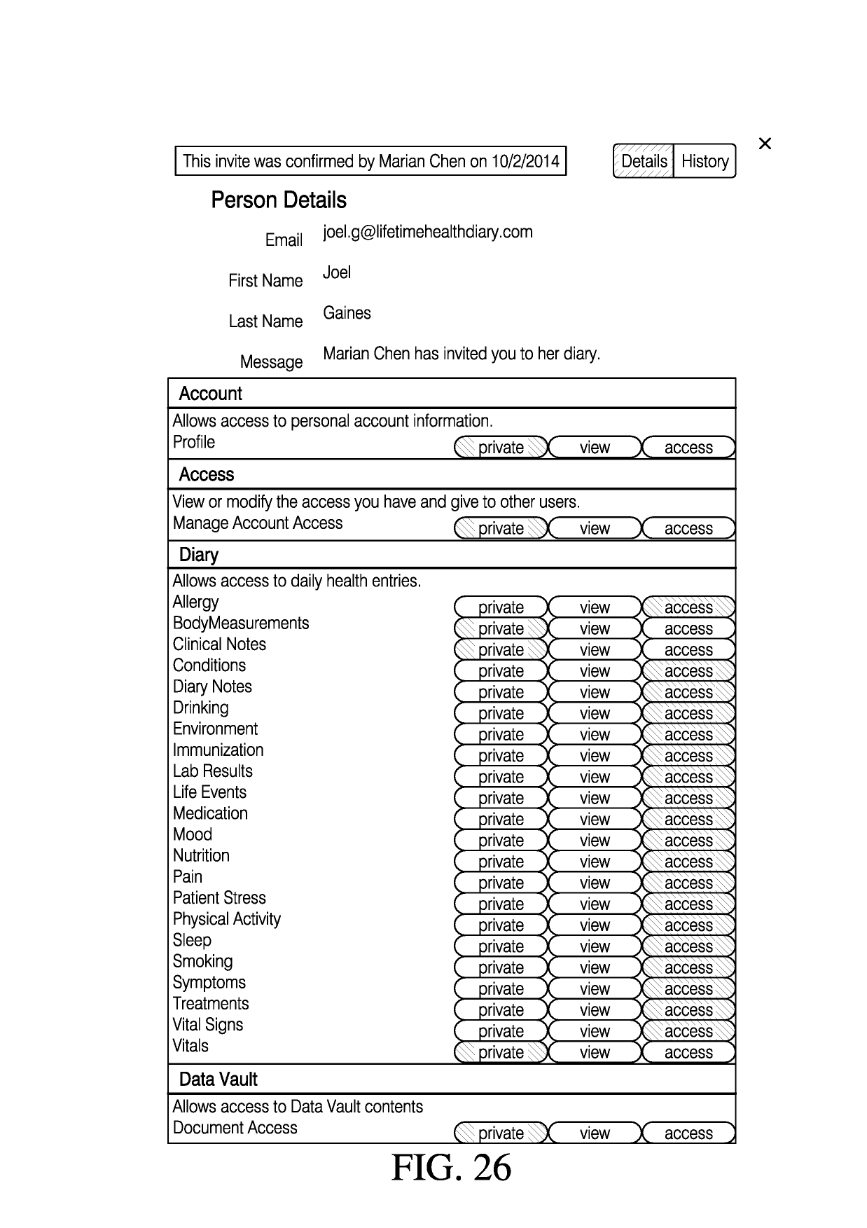

FIG. 26 is an example of a screen display illustrating invitation details retrieved such as using the display of FIG. 23. The invitation can appear in the Invitations Sent panel of the Pending page (see FIG. 23). The details of each invitation can be retrieved and viewed by the owner and by the invitee.

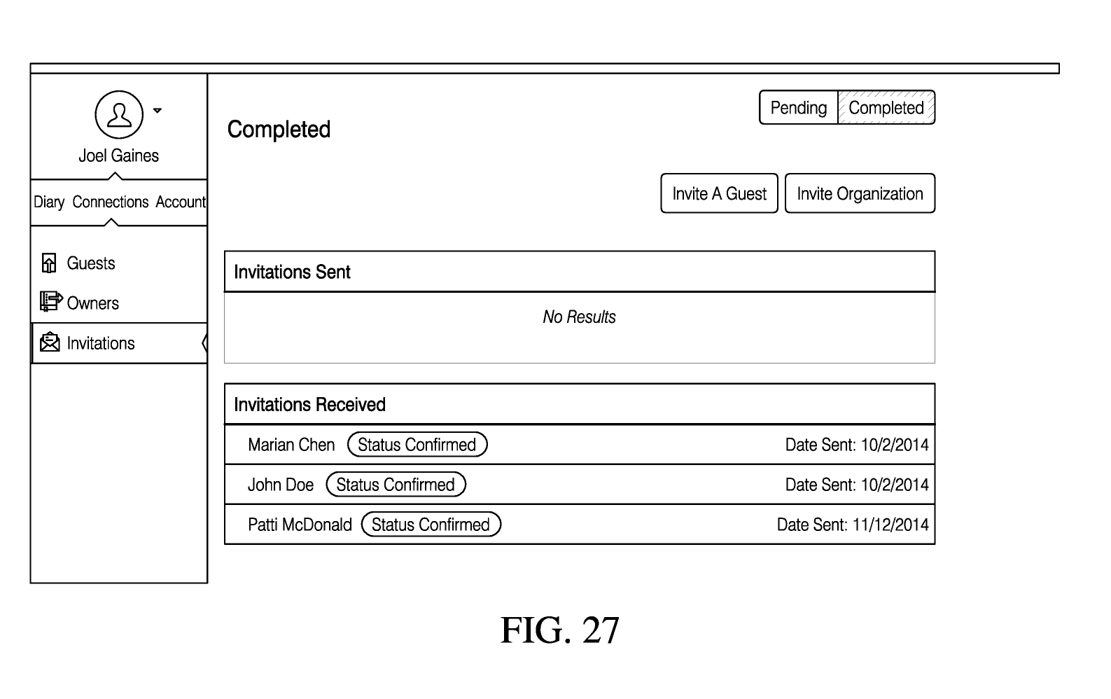

FIG. 27 is an example of a screen display illustrating an invitation completed screen where the completed invitations can be viewed by the owner and guest. Once the invitation process has been completed, the owner and guest can view these invitations as shown in FIG. 27.

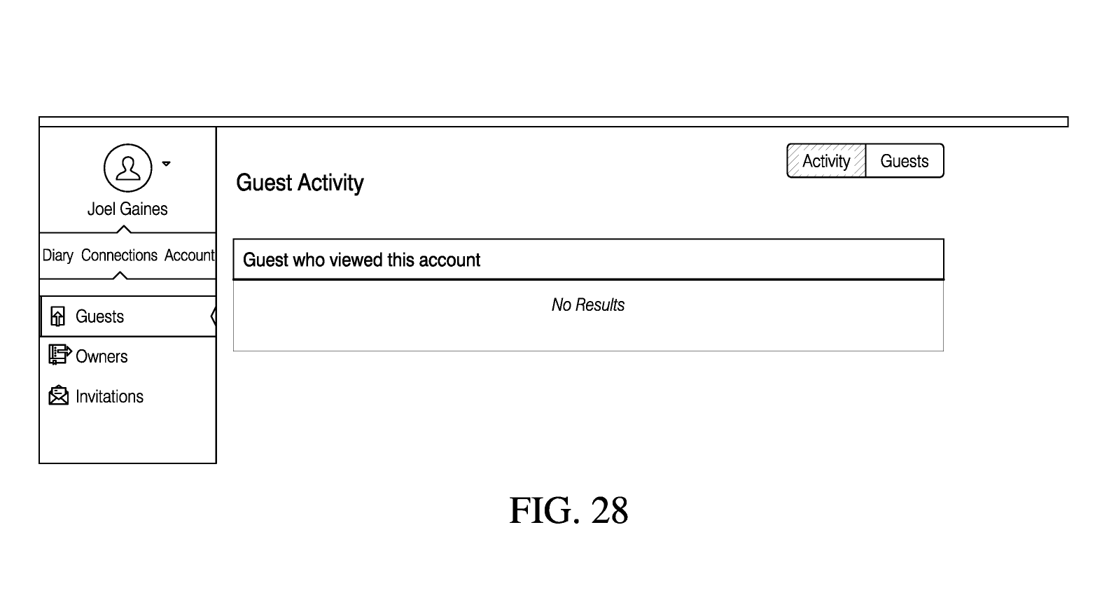

FIG. 28 is an example of a screen display illustrating an audit of activity by a guest on an owner's account. An audit of actions taken by a guest on an owner's account can be viewed by the owner.

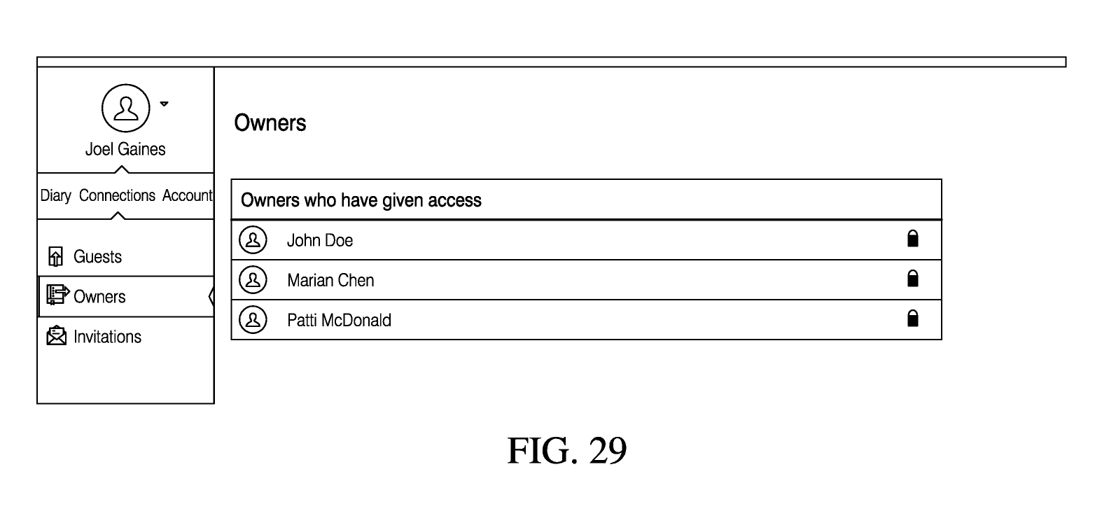

FIG. 29 is an example of a screen display illustrating a summary of owners who have given access to their diaries for a particular guest. A guest can see a summary of owners who have given the guest access to their diaries.

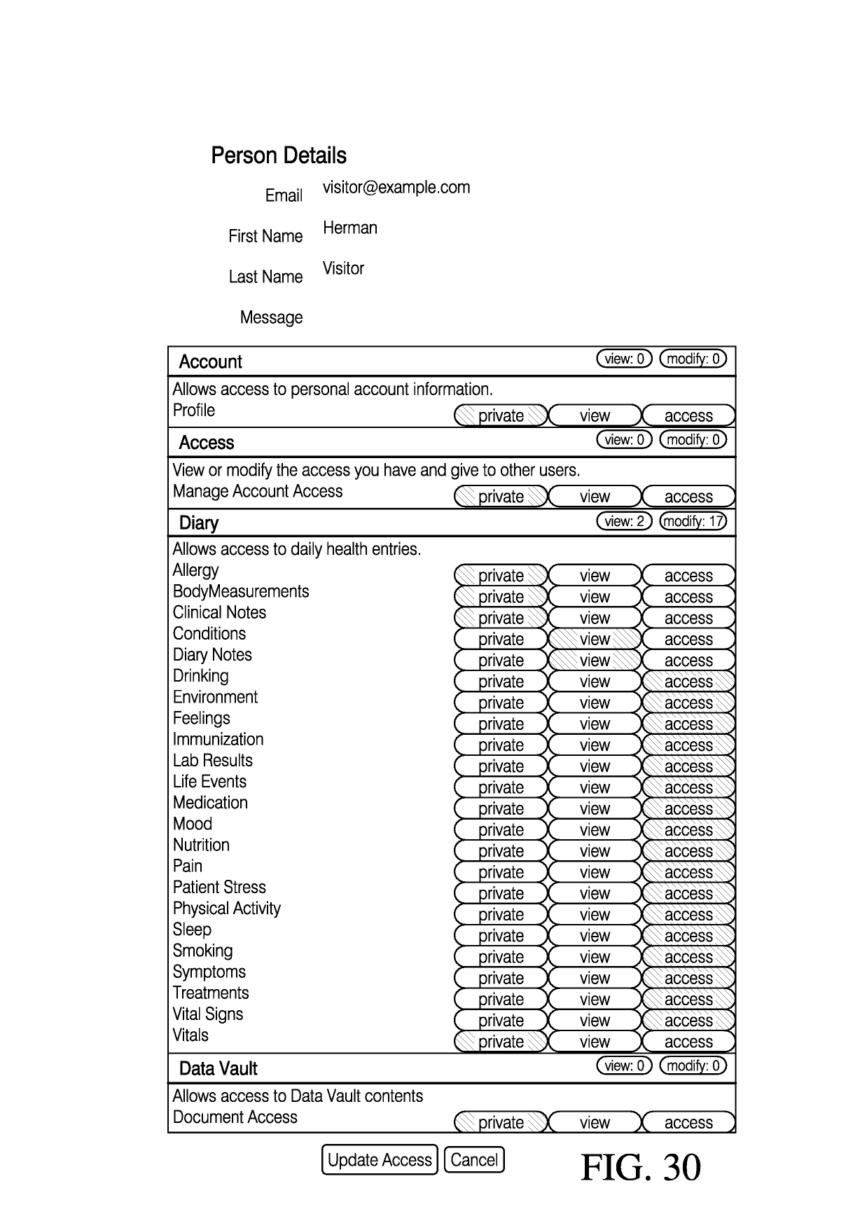

The diary implements an easy to use interface to allow owners to control access to their diary data. FIG. 30 is an example of a screen display illustrating an interface screen seen by a user (e.g., owner) when they wish to update the permissions assigned to a guest. The example of FIG. 30 is the interface seen by a user of the web application when they wish to update the assigned guest permissions. The guest's information is shown at the top of this pop-up dialog. There are four groups of permissions shown in this example—Account, Access, Diary, and Data Vault. Each includes one or more finer-grained permissions. The owner can choose between three settings for each permission as follows:
Private—Data controlled by this permission cannot be accessed by this guest.
View—Data controlled by this permission can only be viewed by this guest (read-only). The guest cannot add, edit or delete any of this data or metadata associated with it.
Access—Data controlled by this permission can be viewed, added, edited or deleted by this guest.

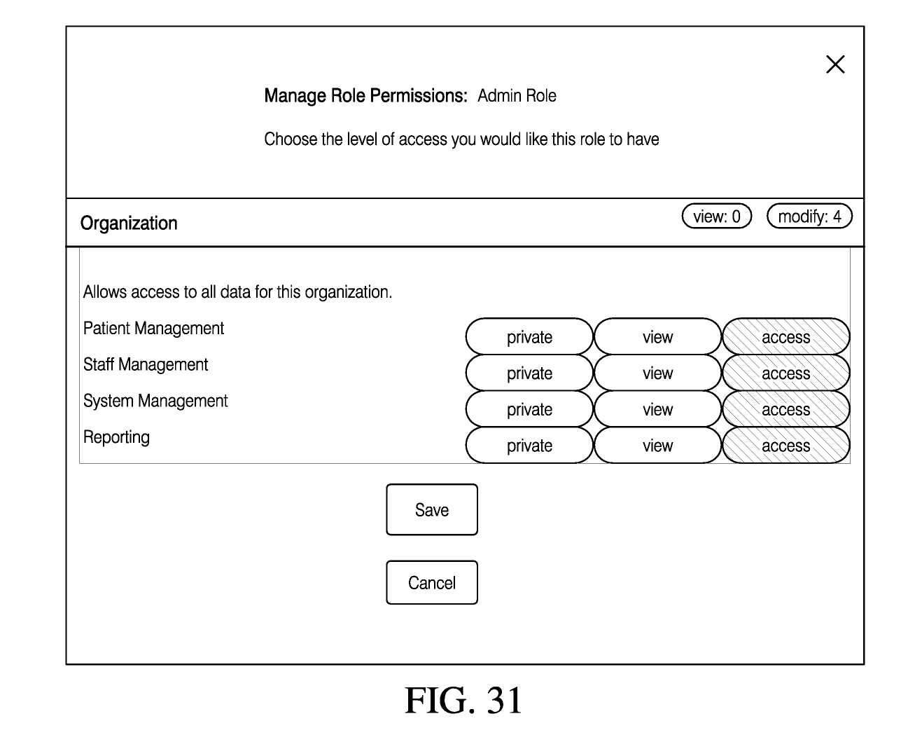

FIG. 31 is an example of a screen display illustrating an interface screen seen by a user (e.g., owner) when they wish to assign permissions to roles in an organization. There are permissions that relate specifically to the administration of organizations. These can only be assigned to roles, so that they can be passed on to staff members.

Organizations can create and delete Access (Patient) Groups and Roles, and control their membership.

### Roles

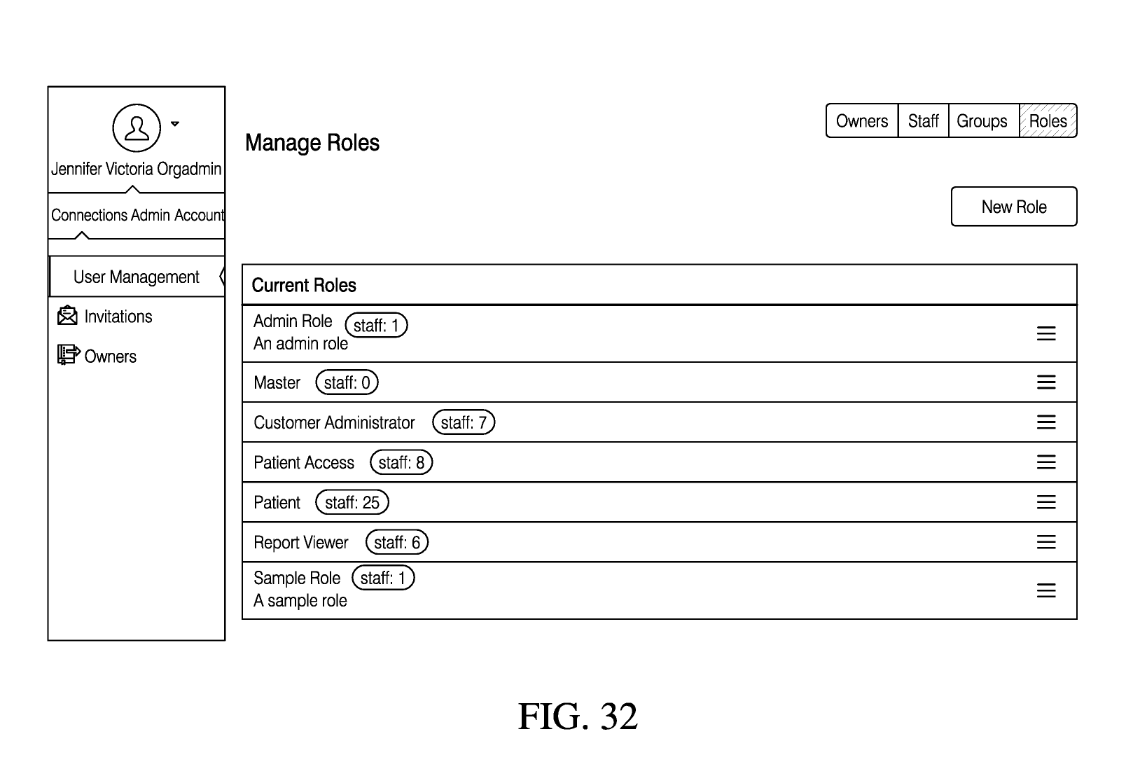

FIG. 32 is an example of a screen display illustrating an interface screen seen by an administrator of an organization when they wish to manage roles in the organization. The figure illustrates an example of a list of roles as can be seen by an organization's administrator. From this page, the administrator can add a new role, delete a role, edit a role's permissions, and choose which staff members belong to a role.

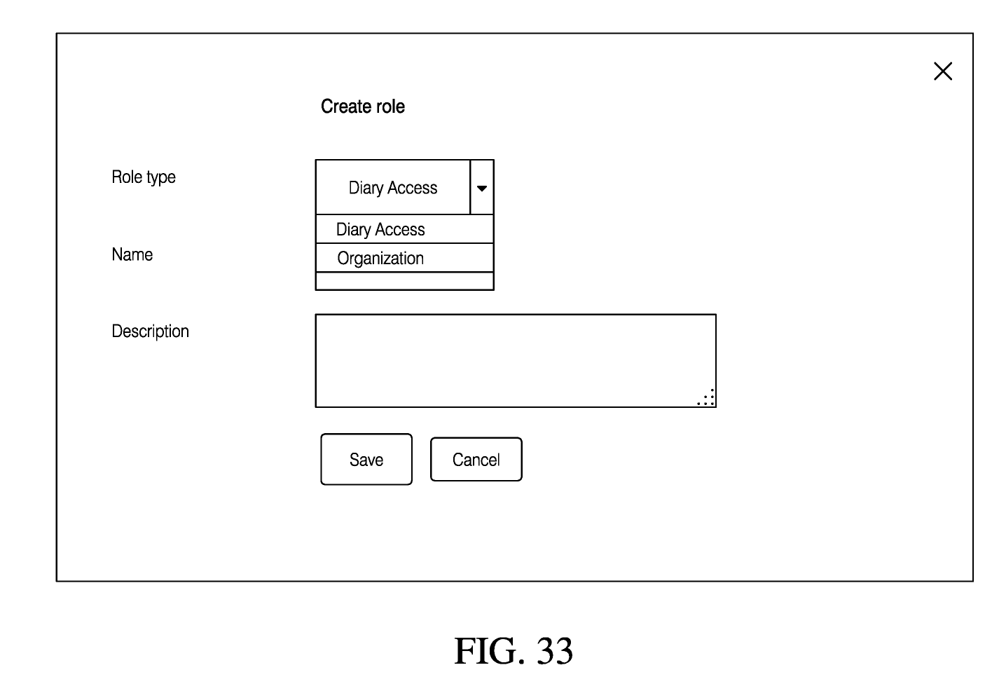

FIG. 33 is an example of a screen display illustrating an interface screen seen by an administrator of an organization when they wish to create a role in the organization. Clicking the new role button shown in FIG. 32 leads to a dialog as illustrated in FIG. 33. In the dialog box, the user can at this point choose a name and enter a description for this new role. The user must choose whether the role is a diary access role (allowing access to owners' diaries depending on permissions), or organization roles (allowing access to administrative functions for this organization). This choice will control which kinds of permissions can be assigned to this role.

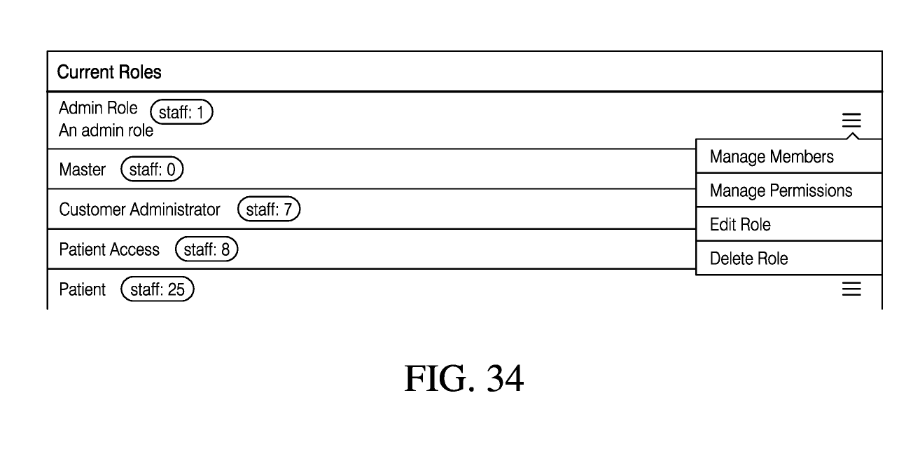

FIG. 34 is an example of a screen display illustrating an interface screen seen by an administrator of an organization of operations that can be performed on corresponding roles in the organization. Clicking on the menu control brings up a menu of operations that can be performed on the corresponding role, such as, for example, managing members, managing permissions, editing a role or deleting a role.

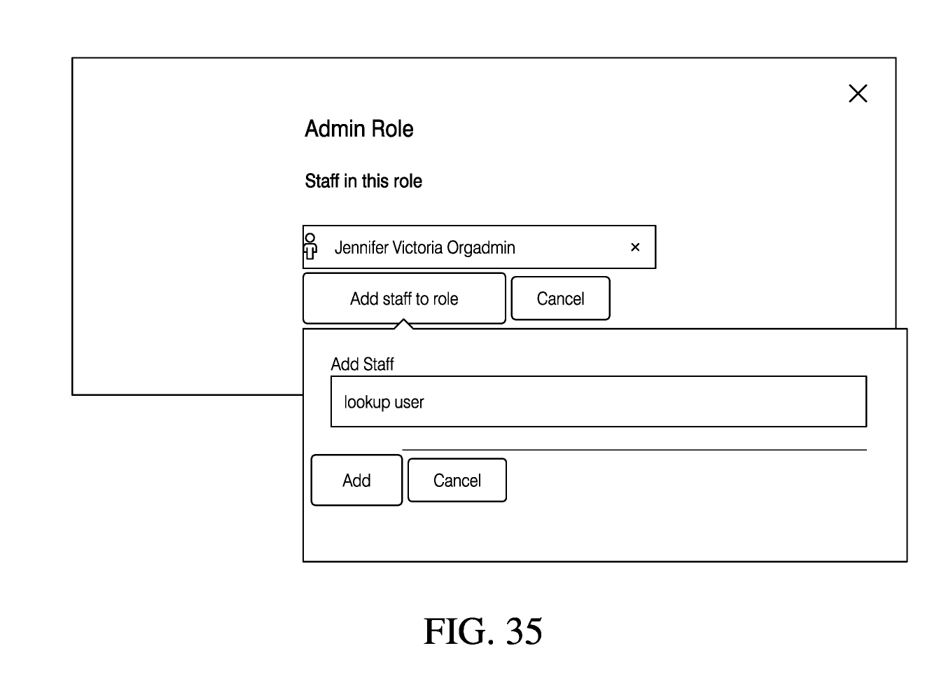

FIG. 35 is an example of a screen display illustrating an interface screen seen by an administrator of an organization in managing staff members. Selecting ‘Manage Members’ in the display of FIG. 34 results in an example dialog as illustrated in FIG. 35. Here it is possible to click the ‘X’ control beside a member to remove them from that role. The ‘Add Staff to Role’ button shows a control that allows the user to locate and select a staff member to add to the role.

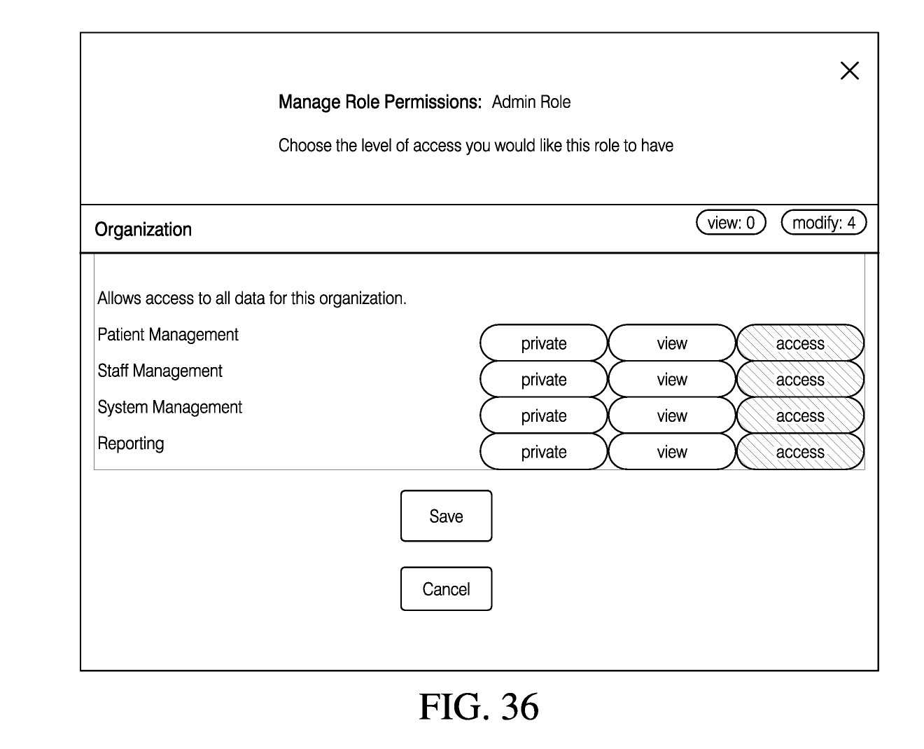

FIG. 36 is an example of a screen display illustrating an interface screen seen by an administrator of an organization to manage role permissions for an organizational role. This is arrived at depending on which option (diary access or organization) is chosen in the dialog illustrated in FIG. 33. A role is for either diary access or organization, and is only able to be allocated suitable permissions. Selecting the menu's ‘Manage Permissions’ item of FIG. 34 results a list of permissions that the organization can choose to delegate to its staff members via membership of this role. In this case, for an organization role example permissions (e.g., access) are as shown in FIG. 36.

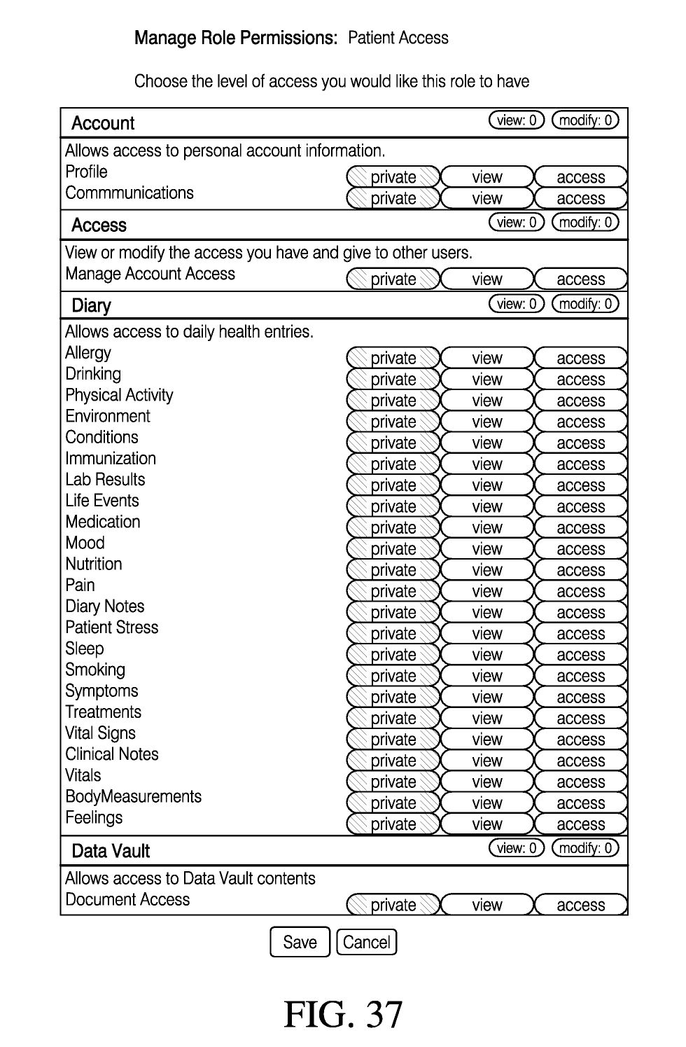

FIG. 37 is an example of a screen display illustrating an interface screen seen by an administrator of an organization to manage role permissions for a diary access role. For a diary access role, example permissions (e.g., private) for the multiple categories of the diary are as shown in FIG. 37.

### Access Groups

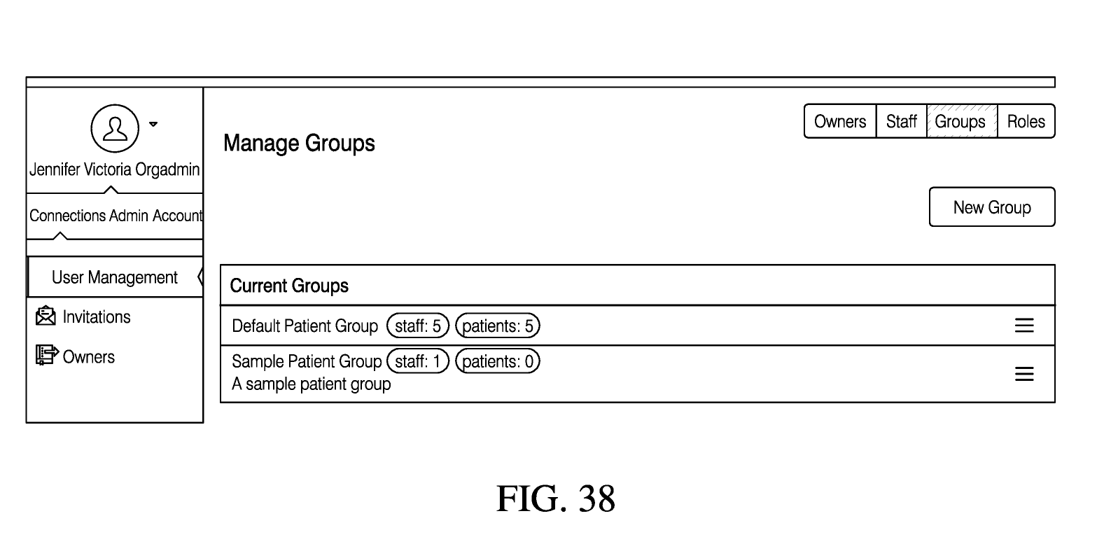

FIG. 38 is an example of a screen display illustrating an interface screen seen by an administrator of an organization to manage owner groups. A list of owner groups is displayed in FIG. 38 as can be seen by an organization's administrator. From this page, the administrator can add a new group, delete a group, and choose which owners and staff members belong to a group.

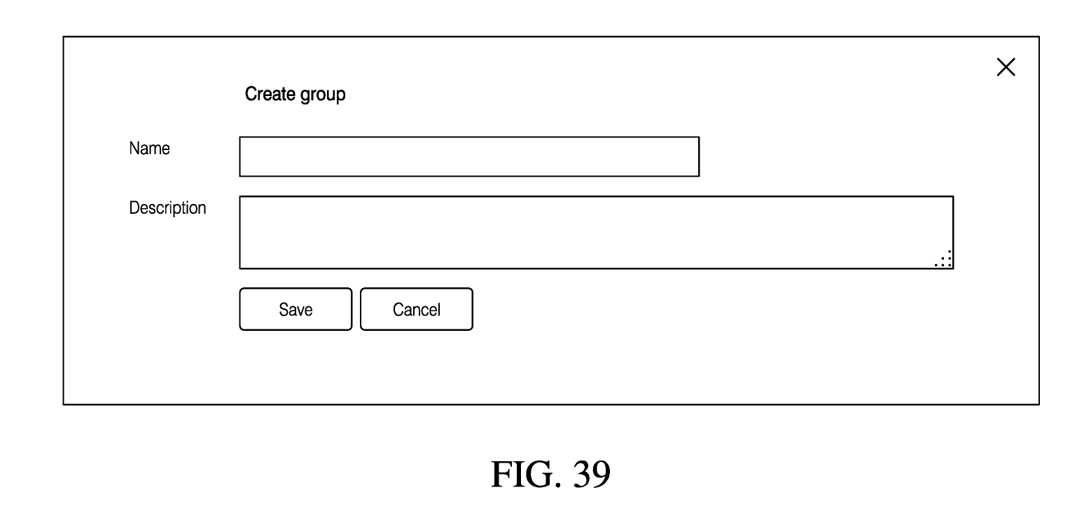

FIG. 39 is an example of a screen display illustrating a screen seen by an administrator of an organization to enter a name and description for a new group. Clicking the ‘New Group’ button in FIG. 38 leads to showing a dialog in FIG. 39 that invites the user to enter a name and description for the new group.

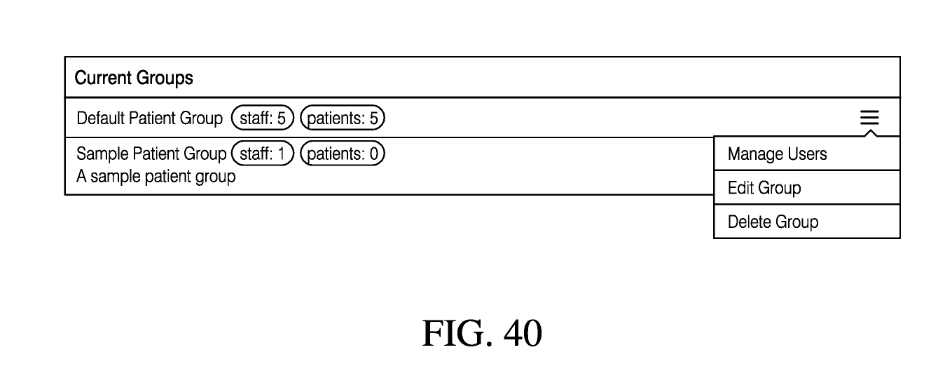

FIG. 40 is an example of a screen display illustrating an interface screen seen by an administrator of an organization for displaying operations that can be performed on a corresponding group. Clicking on the menu control brings up a menu of operations that can be performed on the corresponding role. For example, operations can include: manage users (group), edit group and delete group.

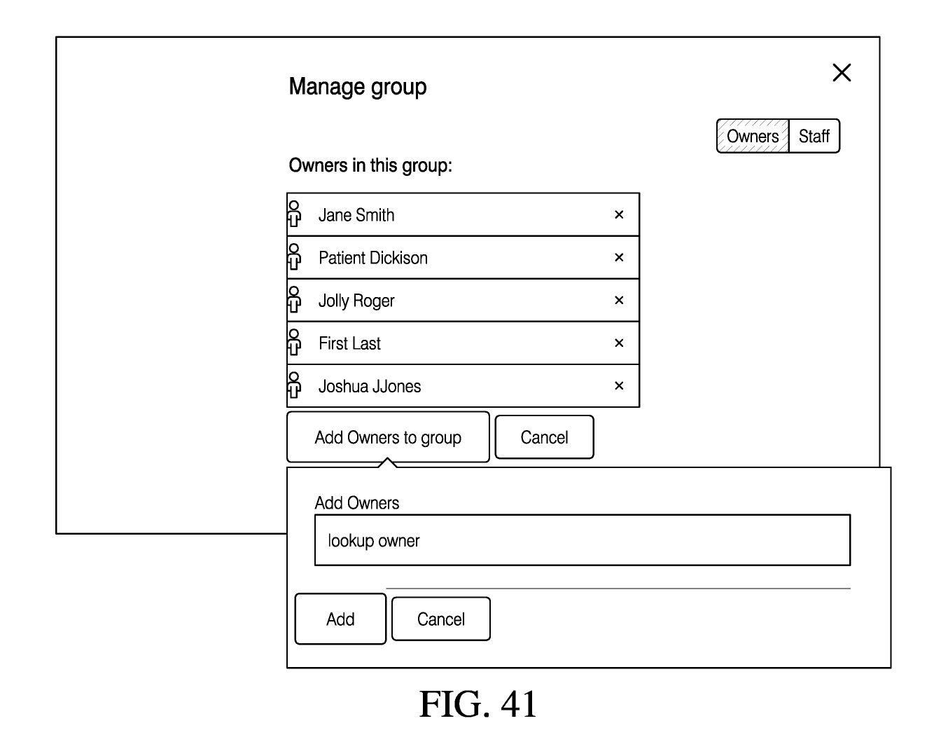

FIG. 41 is an example of a screen display illustrating an interface seen by an administrator of an organization to select owners for a corresponding group. The ‘Manage Group-Owners’ operation allows the user (e.g., organization's administrator) to select which owners are to be in the corresponding group. The display includes an option to look-up additional owners in the system 100.

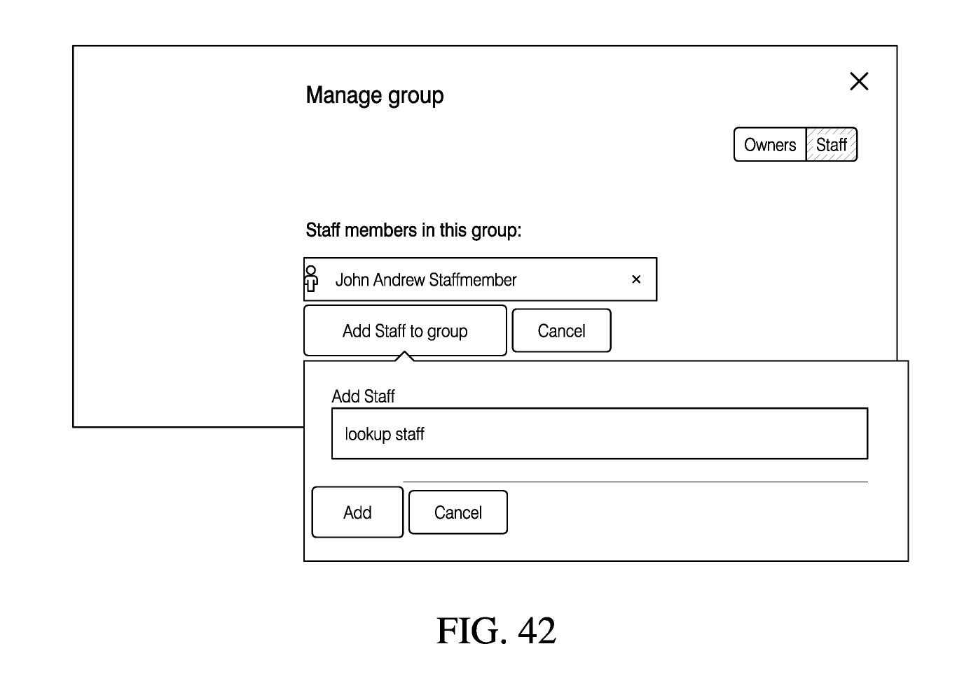

FIG. 42 is an example of a screen display illustrating an interface seen by an administrator of an organization to select staff members for a corresponding group. The ‘Manage Group-Staff’ operation allows the user (e.g., organization's administrator) to select which staff members are to be in the corresponding group. The display includes an option to look-up additional staff in the system 100.

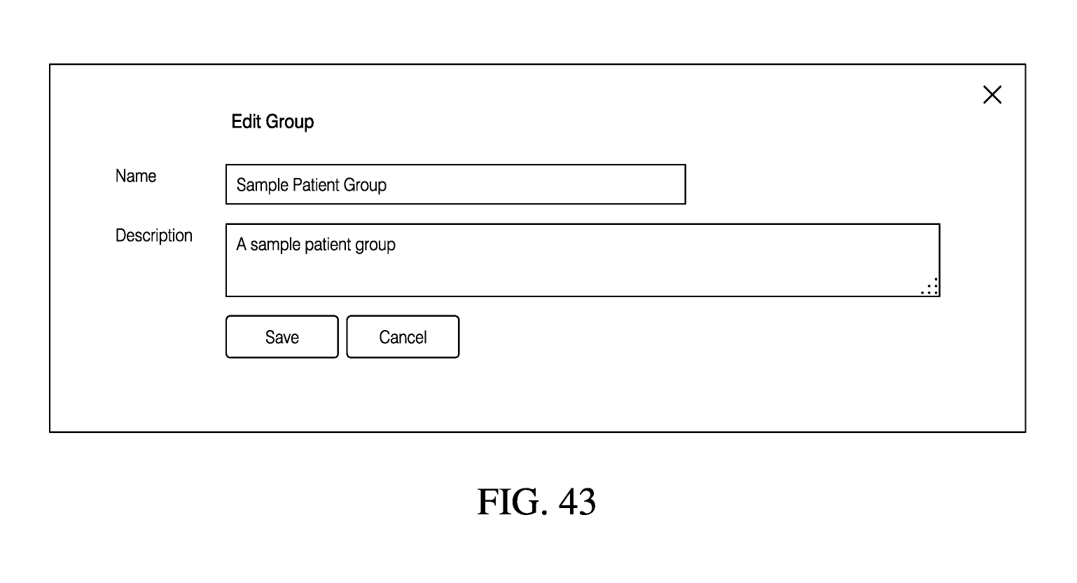

FIG. 43 is an example of a screen display illustrating an interface seen by an administrator of an organization for editing a name and description of a corresponding group. The ‘Edit Group’ operation, as shown in FIG. 40, allows the user (e.g., organization's administrator) to edit the corresponding group, including changing the name and description of the group, for example.

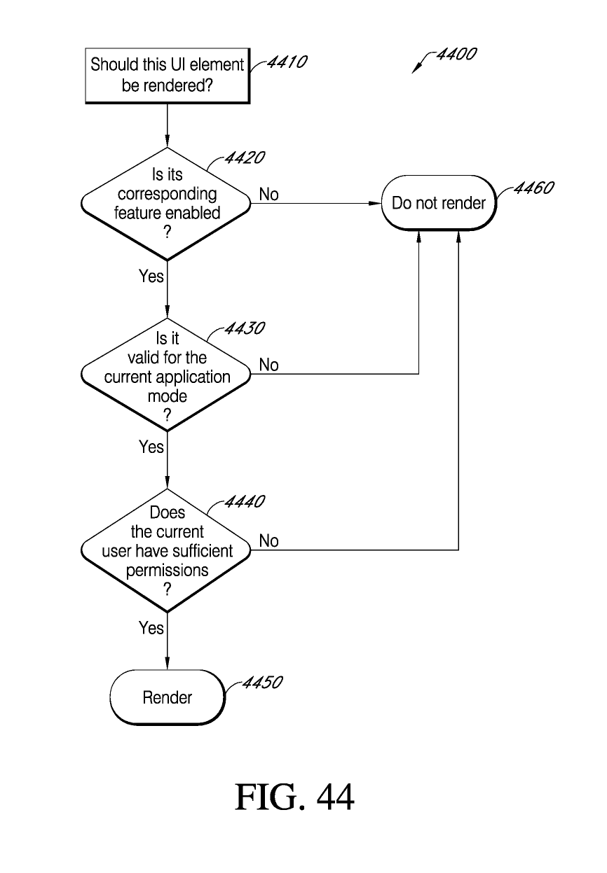

FIG. 44 is a flowchart illustrating an embodiment of a process 4400 for determining whether a user interface element is to be rendered, that is, determining the visibility of a specific user interface element. In certain embodiments, the process can be performed on the system 100 illustrated in FIG. 2A. An XML definitions file (e.g., the sitemap described below) is utilized as part of the process 4400. In certain embodiments, each UI element needs a conditional check as to whether it should be rendered or not.

Beginning at start state 4410 for each user interface (UI) element, process 4400 proceeds to a decision state 4420 to determine if its corresponding feature is enabled. The application has a number of features that can be turned on or off on a per-user basis. In certain embodiments, these features are: language selection, care team and lab result document link, but more features can be added. The assignment of these to users is recorded in a simple link table, PersonApplicationFeature, for example. The check process can be:
Determine whether the given UI element is part of an application feature. If it isn't, this check passes.
Determine whether the current user has been assigned access to the UI element's application feature. If so, the check passes.
Otherwise, the check fails and the process moves to a do not render state 4460.
   The element is only shown if the check passes.

Proceeding to a decision state 4430, process 4400 determines is the UI element is valid for the current application mode. The application context for a user can be in one of a number of modes. This presents the user with a UI that's suited to the functionality required for that mode. In certain embodiments, the modes are: patient, staff and visitor. This system is designed such that it's possible for multiple modes to be supported for a user session at once if that becomes a requirement. The check process is:
If this element is not specific to any mode, the check passes.
If this element's mode corresponds to the user's current mode, the check passes.
Otherwise, the check fails and the process moves to the do not render state 4460.
   The element is only shown if the check passes.

Advancing to a decision state 4440, process 4400 determines whether the current user have sufficient permissions. Each user has a set of permissions to data, including diary (medical/health) data, data vault documents, organization configuration settings, user profile settings, and so forth. In certain embodiments, a user has implied permission to work with their own data, and may have permissions to access and update other users' data. If this element is recorded as being related to a specific type of permission (in the sitemap.xml file previously mentioned), the current user will need to have that permission in order to be able to see the UI element. Permissions determinations for organization members and for a guest are further described hereinbelow. If the user has sufficient permissions process 4400 proceeds to state 4450 to render the UI element. Otherwise, the check fails and the process moves to the do not render state 4460. Process 4400 is repeated for other UI elements so as to complete the user interface.

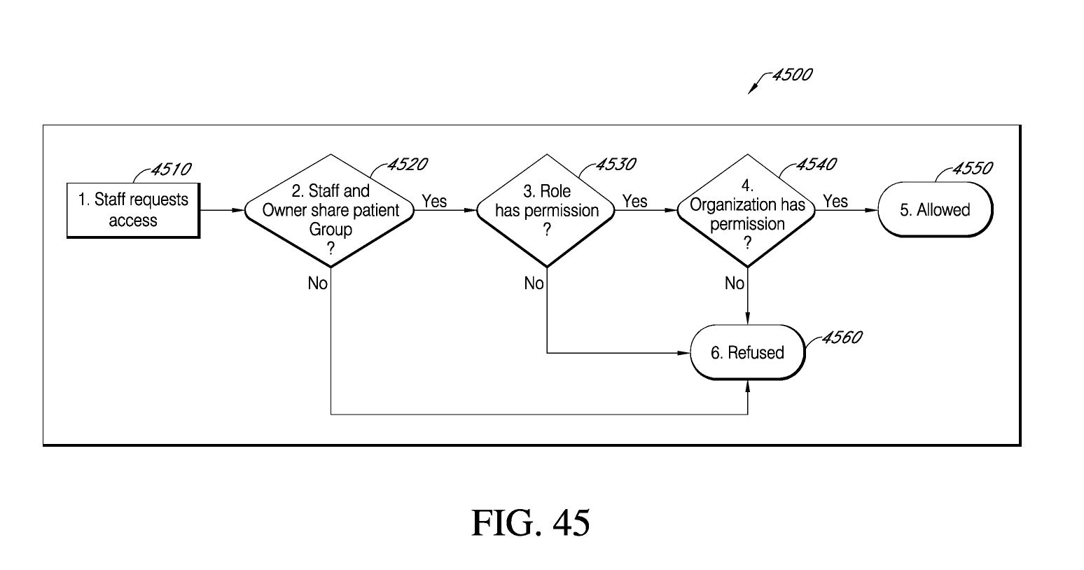

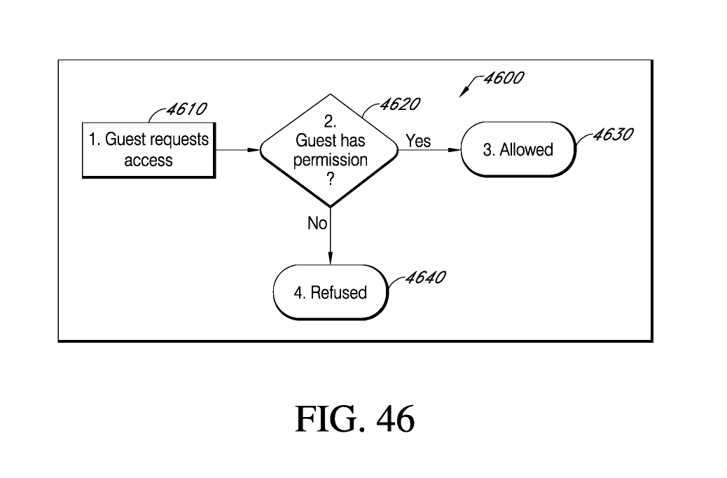

A description of the permissions calculation process is provided in conjunction with FIGS. 45 and 46 described below. This section deals with an example database structure used to store permission-related data. The Permission table in the permissions system defines the set of permissions that can be assigned. This data is generally static. An example Permission table is as follows:

| | | Parent | | |
|---|---|---|---|---|
| Id | Name | PermId | Description | Visible |
| 1 | Organization | NULL | Allows access to all data for this organization. | 1 |
| 2 | Patient | 1 | Allows management of patients. | 1 |
| | Management | | | |
| 3 | Staff Management | 1 | Allows management of staff. | 1 |
| 4 | System | 1 | Covers administration tasks for this organization. | 1 |
| | Management | | | |
| 5 | Reporting | 1 | Allows generation and viewing or reports. | 1 |
| 6 | Patient | NULL | Allows access to all patient data. | 1 |
| 7 | Account | 6 | Allows access to personal account information. | 1 |
| 8 | Profile | 7 | Allows setting up of profile information for a user. | 1 |
| 9 | Communications | 7 | Gives access to a user's communications. | 0 |
| 10 | Access | 6 | View or modify the access you have and give to other | 1 |
| | | | users. | |
| 11 | Manage Account | 10 | Allows maintenance of security and access settings, and | 1 |
| | Access | | sharing of data. | |
| 12 | Diary | 6 | Allows access to daily health entries. | 1 |
| 13 | Allergy | 12 | Covers allergy data. | 1 |
| 14 | Drinking | 12 | Covers drinking data. | 1 |
| 15 | Physical Activity | 12 | Covers physical activity data. | 1 |
| 16 | Environment | 12 | Covers environmental data. | 1 |
| 17 | Conditions | 12 | Covers conditions data. | 1 |
| 18 | Immunization | 12 | Covers immunization data. | 1 |
| 19 | Lab Results | 12 | Covers lab result data. | 1 |
| 20 | Life Events | 12 | Covers life event data. | 1 |
| 21 | Medication | 12 | Covers medication data. | 1 |
| 22 | Mood | 12 | Covers mood data. | 1 |
| 23 | Nutrition | 12 | Covers nutrition data, | 1 |
| 24 | Pain | 12 | Covers pain data. | 1 |
| 25 | Diary Notes | 12 | Covers diary notes data. | 1 |
| 26 | Patient Stress | 12 | Covers patient stress data. | 1 |
| 27 | Sleep | 12 | Covers sleep data. | 1 |
| 28 | Smoking | 12 | Covers smoking data. | 1 |
| 29 | Symptoms | 12 | Covers symptom data. | 1 |
| 30 | Treatments | 12 | Covers treatment data. | 1 |
| 32 | Vital Signs | 12 | Covers vital signs. | 1 |
| 33 | Data Vault | 6 | Allows access to Data Vault contents | 1 |
| 34 | Document Access | 33 | Allows access to documents | 1 |
| 35 | Clinical Notes | 12 | Covers clinical notes data. | 1 |
| 36 | Vitals | 12 | Covers vitals data. | 1 |
| 37 | Body | 12 | Covers body measurements data. | 1 |
| | Measurements | | | |
| 38 | Feelings | 12 | Covers feelings data. | 1 |

The ParentPermId field indicates a parent permission identifier that can be used to identify a permission that encompasses the requested permission. For example, the parent permission identifier of Smoking is Id 12, which is the Diary, and which encompasses Id 28, Smoking. Not all permissions may be usable or visible—the ‘visible’ column controls this.

Permissions can be assigned to other entities via link tables as follows:
ExplicitPermission: Id, PersonId, PatientId, PermissionId, ReadOnly. Used to assign a permission (PermissionId) to use data from a specific Owner (PatientId) to a given user of the Diary (PersonId) with full or read-only access (ReadOnly).
OrganizationExplicitPermission: Id, OrganizationId, PatientId, PermissionId, ReadOnly. Used to assign a permission (PermissionId) to use data from a specific Owner (PatientId) to a given Organization (OrganizationId) with full or read-only access (ReadOnly). Data allowed by this permission can then be accessed by StaffMembers of this Organization when they have been given suitable permissions by the Organization via their Role.
ProposedPermission: Id, InvitationId, PermissionId, ReadOnly. When an invitation is sent (to another user, or to an Organization), a set of proposed permissions is associated with it. This table contains the mapping of permissions to invitation. When the invitation process completes successfully, it is converted to an ExplicitPermission or OrganizationExplicitPermission.
RolePermission: Id, RoleId, PermissionId, ReadOnly. Assigns a permission to a Role. The Role's Organization controls this mapping between Roles and Permissions. It also controls which Staff Members are members of which Roles, and which Staff Members belong to which Patient Group.
PatientGroup: Id, Name, OrganizationId, UID, Description. A PatientGroup belongs to a single Organization (OrganizationId). The Organization controls which Patients (Owners) belong to each Patient Group.
PatientToPatientGroupLink: PatientId, PatientGroupId. A link table that records the assignment (performed by the Organization that owns the Patient Group) of Patients (Owners) to Patient Groups.
StaffMemberToPatientGroupLink: StaffMemberId, PatientGroupId. A link table that records the assignment (performed by the Organization that owns the Patient Group) of Staff Members to Patient Groups. A Staff Member can only access data of an Owner (Patient) if they share a common Patient Group.
StaffMemberToRoleLink: StaffMemberId, RoleId. A link table that records the assignment (performed by the Organization that owns the Role) of Staff Members to Roles. A Staff Member can only access data of an Owner (Patient) if they inherit from their Role a suitable permission for the data they want to access.

### Permissions Hierarchy

In certain embodiments, the permissions hierarchy is as follows:

Organization
Patient Management
Staff Management
System Management
Reporting

Patient
Profile
   Communications
   Account
Access
   Manage Account Access
Diary
   Allergy
   Drinking
   Physical Activity
   Environment
   Conditions
   Immunization
   Lab Results
   Life Events
   Medication
   Mood
   Nutrition
   Pain
   Diary Notes
   Patient Stress
   Sleep
   Smoking
   Symptoms
   Treatments
   Vital Signs
   Clinical Notes
   Vitals
   Body Measurements
   Feelings
Data Vault
   Document Access

### Permissions Calculations

When a user of the diary wants to view or change data in an owner's diary, the application needs to check whether the current user has permission to do so. A diary permission specifies the kind of data that is to be accessed, and whether that access only requires viewing, or whether it involves making changes to the diary (creating, updating, or deleting records). Once the application calculates what kind of data is to be accessed, and whether the user is editing vs. only viewing, it can follow the processes below to determine whether the access should be allowed. A user will be acting in the context of a staff member or a guest, and the process differs based on this distinction.

FIG. 45 is a flowchart illustrating an embodiment of a process 4500 for determining whether a particular staff member of an organization has access to an owner's data based on permissions. In certain embodiments, the process can be performed on the system 100 illustrated in FIG. 2A. This process 4500 is followed when a staff member of an organization (e.g., doctor) tries to access any piece of diary data from an owner.

Beginning at a start state 4510 when a staff member of a given organization attempts to access an item of data from an owner's diary, process 4500 moves to a decision state 4520 where a determination is made whether the staff member and the owner share an access group, which in certain embodiments is a patient group. The staff member must be assigned to an access group that contains the given owner (patient). If not, the organization has not authorized the staff member to access that group (and therefore that patient), so permission is refused at state 4560.

Proceeding to a decision state 4530, process 4500 determines if the role(s) have permission. For each role to which the staff member belongs, check whether the organization has given a permission that matches the request. This means that either the specific permission has been given, or any permission that encompasses the requested permission has been given, such as described above. If no role has permission, access is refused at state 4560.

Advancing to a decision state 4540, process 4500 determines if the organization has permission. A check is made as to whether the owner has given the particular organization a permission that matches the request. This means that either the specific permission has been given, or any permission that encompasses the requested permission has been given. If the organization does not have permission, access is refused at state 4560. If all prior requirements at decision states 4520, 4530 and 4540 have been fulfilled, the staff member is granted access by both the owner and the organization. Therefore, they are allowed to view the requested data. Note that the checks at decision states 4520, 4530 and 4540 can be performed in any order.

FIG. 46 is a flowchart illustrating an embodiment of a process 4600 for determining whether a particular guest has access to an owner's data based on permissions. In certain embodiments, the process can be performed on the system 100 illustrated in FIG. 2A. This process 4600 is followed when a guest tries to access any piece of diary data from an owner.

Beginning at a start state 4610 when a guest attempts to access an item of data from an owner's diary, process 4600 moves to a decision state 4620 where a determination is made whether the guest has permission. Process 4600 check whether the guest has been given a permission by the owner that matches the request. This means that either the specific permission has been given, or any permission that encompasses the requested permission has been given. If the guest has permission as determined at the decision state 4620, process 4600 proceeds to state 4630 and the guest is granted access by the owner. The guest is allowed to view the requested data. If the guest does not have permission as determined at the decision state 4620, process 4600 proceeds to state 4634 and access is refused. This process 4600 for a guest is simpler than that for a staff member, because permissions are given directly by an owner to a guest, and form links between them.

### Implementation Details and Dynamic User Interface

Permission-based security is implemented in multiple layers. In certain embodiments, it is intended that the user interface only shows options that are available to the user. e.g., if the current user has no permission to see the data vault, the data vault control in the navigation bar should not be visible.

In certain embodiments, the site layout is defined by a flexible sitemap, defined in XML. An example sitemap (simplified) is as follows:

<sitemap>
<group title=″Diary″ resourcekey=″Navigation_Diary″ modes=″Patient″>
<item title=″Pulse″ resourcekey=″Navigation_Pulse″ controller=″Pulse″
action=″Index″ area=″PatientDiary″ cssclass=″pulse″>
<systemmoduleitems />
</item>
item id=″Timeline″ title=″Timeline″ resourcekey=″Navigation_Timeline″
controller=″Timeline″ action=″Index″ area=″PatientDiary″
cssclass=″timeline″ />
<item title=″Data Vault″ resourcekey=″Navigation_DataVault″
controller=″DataVault″
action=″Index″ area=″PatientDiary″ cssclass=″datavault″ />
</group>
<group title=″Connections″ resourcekey=″Navigation_Connections″>
<item title=″User Management″ controller=″Users″ action=″Index″
area=″Organization″ modes=″Staff″ permission=″StaffManagement″
cssclass=″user-management″>
<linkeditem controller=″PatientGroups″ action=″Index″ area=″Organization″ />
<linkeditem controller=″Roles″ action=″Index″ area=″Organization″ />
<linkeditem controller=″Staff″ action=″Index″ area=″Organization″ />
</item>
<item title=″Invitations″ controller=″Invitation″ action=″Index″
area=″Organization″ modes=″Staff″ permission=″PatientManagement″
cssclass=″invitations″>
<linkeditem controller=″Invitation″ action=″Completed″ area=″Organization″
/>
</item>
<item title=″Owners″ controller=″StaffProfile″ action=″Owners″
area=″Organization″
modes=″Staff″ permission=″PatientManagement″ cssclass=″owners″/>
<item title=″Guests″ controller=″AccessGiven″ action=″Index″
area=″PatientAccount″
modes=″Patient″ permission=″PatientAccess″ cssclass=″guests″>
<linkeditem controller=″AccessGiven″ action=″Friends″
area=″PatientAccount″ />
</item>
<item title=″Owners″ controller=″AccessReceived″ action=″Index″
area=″PatientAccount″ modes=″Patient″ permission=″PatientAccess″
cssclass=″owners″ />
<item title=″Care Team″ resourcekey=″Navigation_CareTeam″
controller=″CareTeam″
action=″Index″ area=″PatientDiary″ cssclass=″careteam″
requiresenabledfeature=″CareTeam″ />
</group>
<group title=″Admin″ permission=″StaffManagement″ modes=″Staff″ >
<item title=″Organization Profile″ controller=″Profile″ action=″Index″
area=″Organization″ permission=″SystemManagement″ cssclass=″organization-
profile″ />
</group>
<group title=″Account″ resourcekey=″Navigation_Account″>
<item title=″Profile″ controller=″Profile″ action=″Index″ area=″Visitor″
modes=″Visitor″ cssclass=″profile″>
<linkeditem title=″Password″ controller=″Profile″ action=″ChangePassword″
area=″Visitor″ />
</item>
<item title=″Profile″ controller=″MinimalProfile″ action=″Index″
area=″PatientAccount″ modes=″Patient″ permissionabsent=″Profile″
cssclass=″profile″ />
<item title=″Notification″ controller=″Notification″ action=″Index″
area=″PatientAccount″ modes=″Patient″ permission=″Profile″ disabled=″true″
cssclass=″notification″ />
<item title=″Preferences″ controller=″Settings″ action=″Index″
area=″PatientAccount″ modes=″Patient″ permission=″Profile″
cssclass=″preferences″ />
</group>
</sitemap>

The sitemap defines when each part of the user interface should be shown. For each item, it defines the title text, the corresponding action (via the Area, Controller, and Action attributes), and in which situations it should be shown. The terms Area, Controller, and Action are all ASP.Net MVC standard terms and are used such as follows: <item title=“User Management” controller=“Users” action=“Index” area=“Organization” modes=“Staff” permission=“StaffManagement” cssclass=“user-management”>. The core application functionality is split up into areas. An example of an area (shown here) is ‘Organization’. This area contains all functionality that is specific to Organization users. In this case, this item includes management of users (for this Organization)—Patient Groups, Roles, and Staff. If the current login isn't operating in an organizational context, this item won't be shown, as the Organization area isn't relevant. ‘Users’ is the name of this particular controller within the Organization area, and Index is the action on this controller. The Users controller is a manager for all user-based functionality within the Organization area. Index is a particular action on this controller; in this case, it's the main, default page that's shown when a user enters this area of the application. In summary, if the current user is a staff member (modes=“Staff) with the StaffManagement permission (permission=“StaffManagement”), they can see the action ‘index’ on the controller ‘users’ in the area ‘organization’.

‘Modes’ control visibility based on the type of user currently logged in, for example:
Patient: an owner is viewing their own diary;
Visitor: a guest is viewing an owner's Diary;
Staff: a staff member is using the application, and may be able to see organization administration functions (with suitable permissions).

Visibility is also controlled by the presence of permissions. The ‘permission’ and ‘permissionabsent’ attributes can be used to show a user interface feature only when a given permission is present or absent, respectively. Also note the ‘requiresenabledfeature’ attribute, which controls the visibility of user interface components based on whether certain application features are currently enabled.

### Back-End Permission Enforcement

In addition to modifying the UI in order to present only the relevant feature set to the user, the back end enforces permission restrictions. This is achieved mainly by application of attributes to MVC Controllers in an ASP.NET framework, such as in the following example:

```
[PatientRequired]
  [PatientAuthorize(PermissionsIdentity.Profile, true) ]
  public class PatientProfileController:
PasswordEnabledMultiModelDomainController<Patient, PatientListViewModel,
PatientSummaryViewModel, PatientSummarvViewModel>
  {
...
}
```

The PatientRequired attribute enforces the requirement that the current user must have a current patient context. An owner viewing their own diary, or a guest viewing another owner's diary, satisfies this requirement. A staff member whose only permissions are organizational administration related permissions would not be operating within a patient context, so would be refused access in this example.

The PatientAuthorize attribute specifies which permission the current user must have for the current diary in order to proceed. In this case, the ‘Profile’ permission must be present. Additionally, it is specified that the user must have full permissions, read-only is not sufficient (the second parameter, ‘true’ in this example).

Additionally, it is possible for certain actions on a MVC controller to require full access when the controller overall only requires read-only permission. See the following example:

[PatientAuthorize(PermissionsIdentity.LabResults, false)]
public class LabResultController :
PatientDataControllerBase<PatientTestResultLog,
LabResultListModel, LabResultViewModel, LabResultViewModel>
{
...
public override ActionResult Index( )
{
return View(UserApplicationContext.State.SelectedPatient.PatientId) ;
}
[NoCache]
[HttpGet]
[LHDAuthorizeRequireWrite]
public override ActionResult Edit(int id)
{
return HttpNotFound( ) ;
}
...
}

In the above example, access to the LabResultController's actions will by default require only the ‘LabResults’ permission, whether read-only or full access. However, for the Edit action to be accessible to a user, the full-access ‘LabResults’ permission must be present; the read-only version is not sufficient and will result in the action being refused.

### Updating the User Interface Based on Permissions

The view of an owner's diary can be different depending on who is viewing it. The owner of the diary can see the entire diary without restriction. Users viewing a diary via an invitation (e.g., guests and staff members of organizations) may have their view restricted to a subset of the diary by the permissions set by the owner (and by the organization, for staff members). This manifests in a different user interface (UI) experience in each case.

### Effects on the User Interface

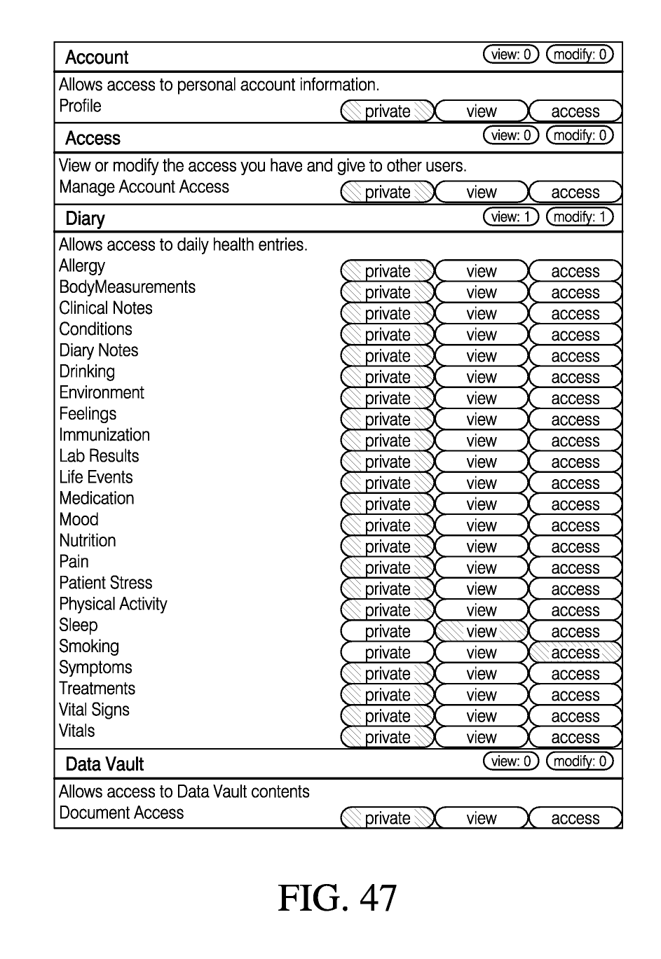

FIG. 47 is an example of a screen display illustrating an interface screen seen by the owner who has set certain permissions as illustrated. In the example of FIG. 47, the owner has set certain permissions. Note that access is given only to the Sleep and Smoking modules. Sleep is read-only, and full write access is given to Smoking. When this guest chooses to view this diary, the options they see in the UI are limited.

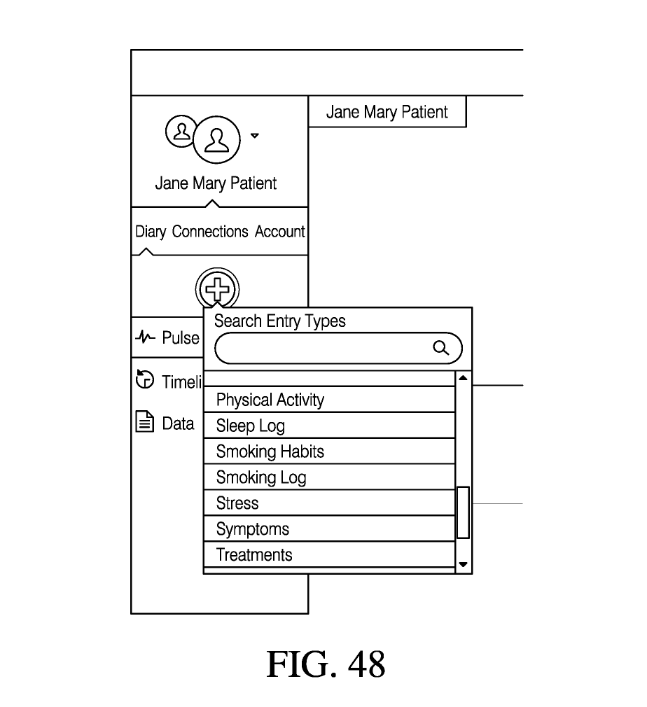

FIG. 48 is an example of a screen display illustrating an interface to display a list of data modules when the owner clicks the ‘Plus’ icon to see to which modules they can add data. FIG. 48 illustrates a portion of an example list shown when the Owner clicks the ‘Plus’ icon. All modules are shown in the dropdown list illustrated in FIG. 48. In other embodiments, the Plus icon opens an entry screen, such as for entering new data.

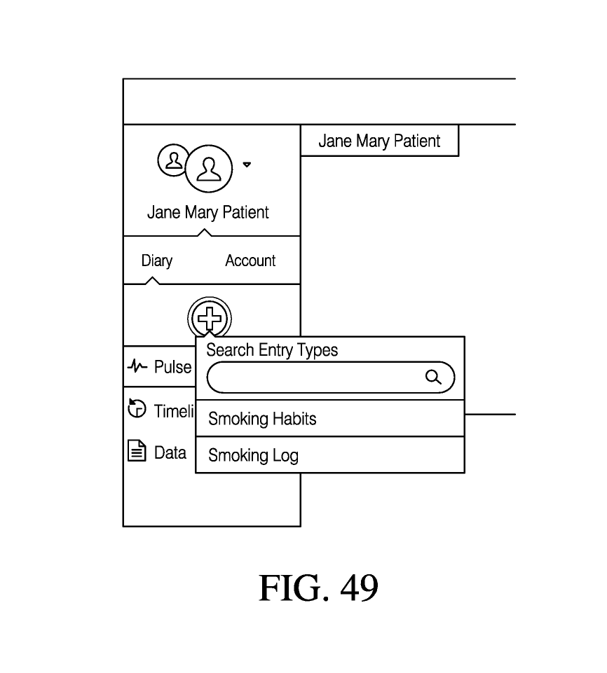

FIG. 49 is an example of a screen display illustrating an interface to display a list of modules restricted to relevant items when a guest performs the same action for the diary of FIG. 48. All modules are shown in the dropdown list illustrated in FIG. 48. However, if the guest performs the same action for this diary, their permissions only allow them to enter data for the Smoking modules, so the list is restricted to the relevant items.

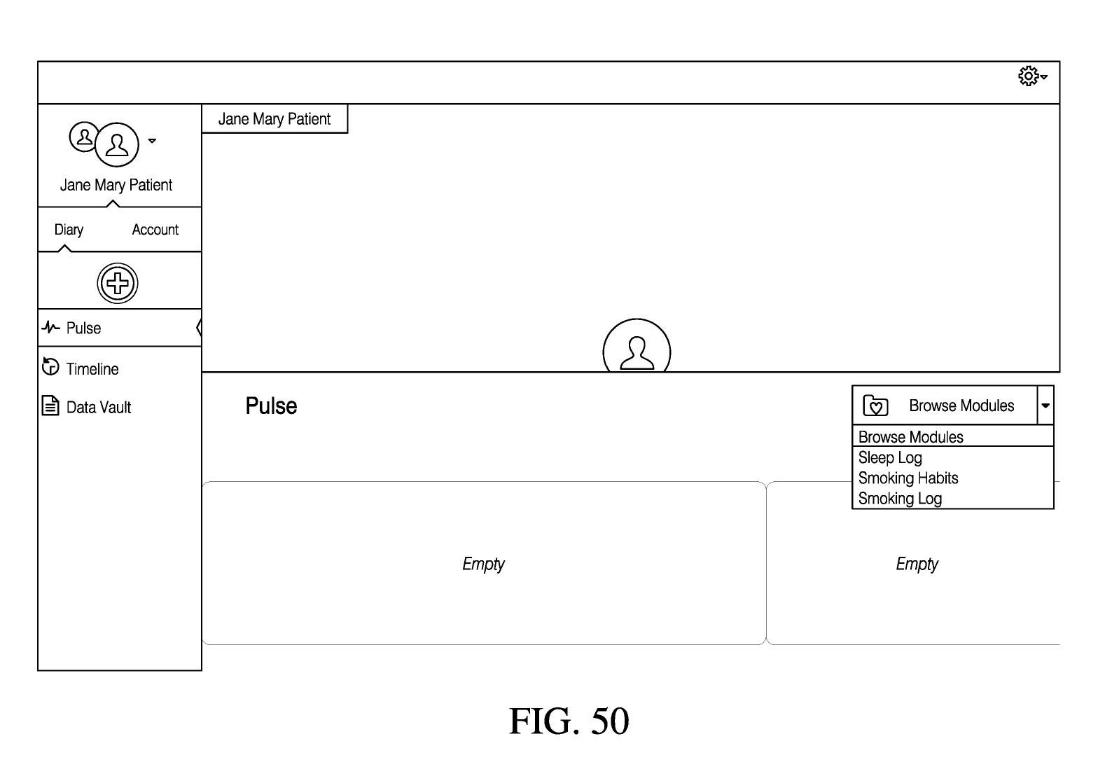

FIG. 50 is an example of a screen display illustrating an interface for a pulse page seen by the guest for a diary. When viewing data from modules, a similar rule is applied. In certain embodiments, the pulse page is a dashboard for an owner's diary, which allows users to place the important aspects of their diary on one page. This is so owners and guests can glance at what is relevant to them without having to navigate throughout the application. On the pulse page seen by the guest for this diary in FIG. 50, only modules for which the guest has view permissions can be selected.

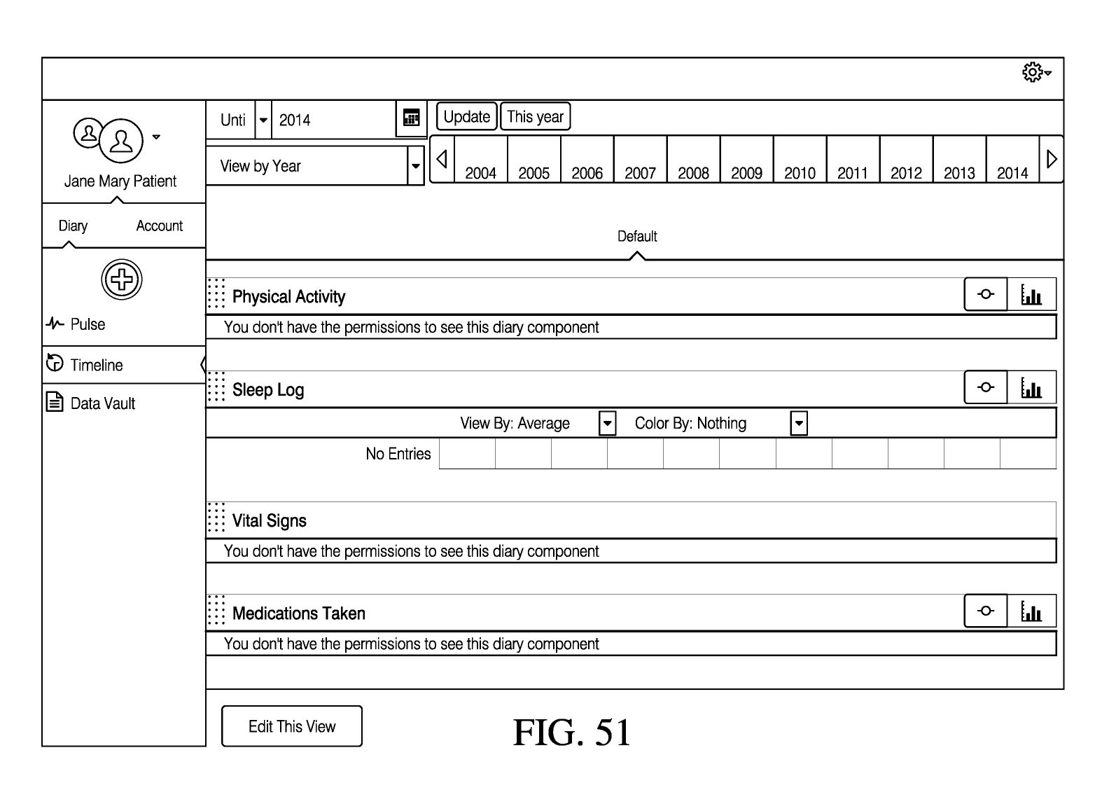

FIG. 51 is an example of a screen display illustrating an interface screen for a timeline which has been chosen but which cannot retrieve data for all modules due to permission limitations. In the timeline view of FIG. 51, any timeline module which has been chosen but which cannot retrieve data due to permission limitations displays a message informing the user that certain data is not available to them.

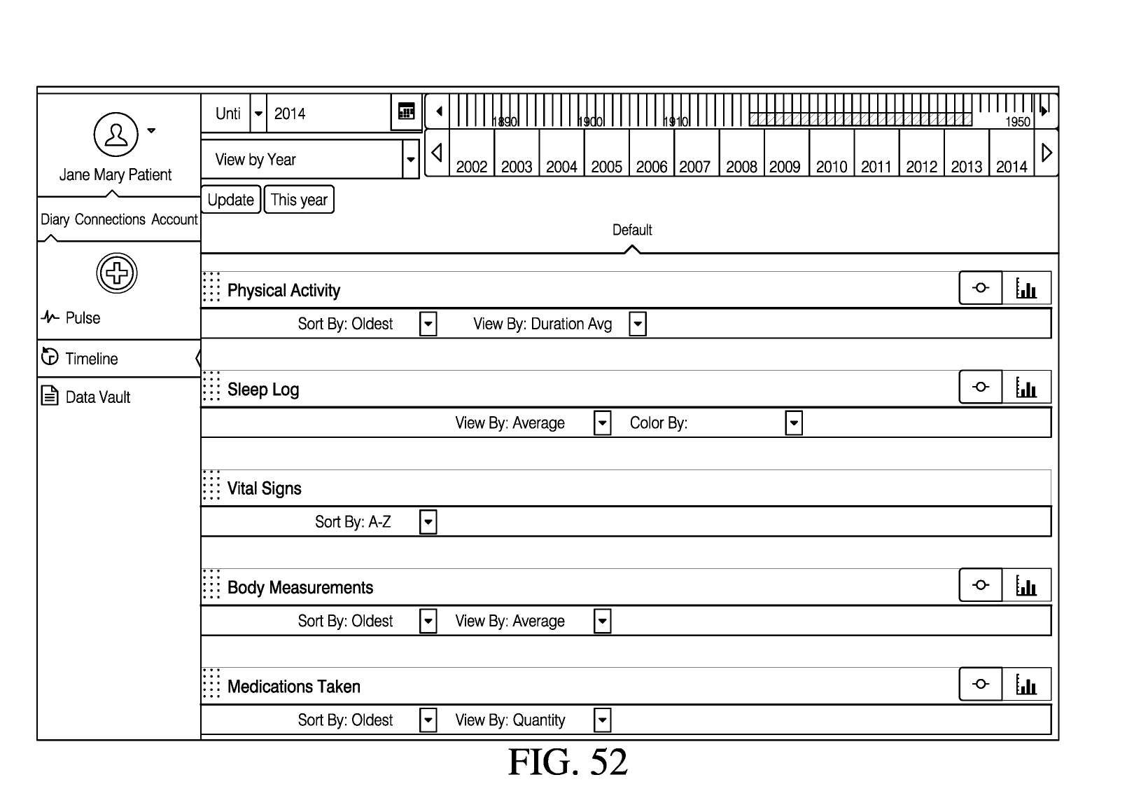

FIG. 52 is an example of a screen display illustrating an interface screen for a timeline page showing a time-based summary of an owner's data. The user can choose which modules to view (subject to permissions if the user is not the owner). The timeline page shows a time-based summary of an owner's data. The data is shown in a linear calendar-like view, with time on the X axis, and different modules (and the data within them) on the Y axis.

The timeline is a temporally correlated, aggregated view of the data that comprises a specific diary. The viewport (currently displayed region) of the timeline can be zoomed in and out (e.g., by day, week, month, and year) and also panned back and forward through time. Any subset of the diary data can be selected for viewing and in the order that suits the needs of the viewer. In certain embodiments, modules can be arranged in an order selected by the user, such as, for example, drag and drop re-ordering or other ways to re-order.

Timelines provide an ability to identify correlations between different data types, and provide an ability for a viewer to rapidly familiarize themselves with the condition/history of the individual the diary represents. Timelines also provide an ability for the data display to be easily configured to meet the goals/interests of the current viewer, and provide an ability to easily/quickly navigate to any specific data at any point in the health history.

Typically, patient medical information is collected using XML data export files into a comma separated values (CSV) file, and then manually displayed in a spreadsheet. Different views of such a spreadsheet makes temporal correlation extremely challenging as the dates can be out of order if sorted by drug name, as are the dosages and any change in medication. On the other hand, other sort orders will mean that drug names are jumbled up, making the view even more confusing. The overall effect of the state of the art is to make decisions and judgment time consuming, non-intuitive, complicated, and usually requiring specific expertise.

By contrast, set forth in FIGS. 51 and 52 are examples of display formats that provide relevant data in a manner that is especially intuitive and helpful for all care team members. The timeline displays each module for the owner and modules for which a guest or a staff member at an organization have been granted permissions. Each module can have additional controls, such as list view or graph view, and drop-down menus such as for example: 1) Sort by, 2) View by and 3) Color by.

The module data may be displayed using a consistent set of symbols to denote physiological measurements, drugs, dosages, starts and stops, and so forth. In certain embodiments, each line contains data for one variable (e.g., a drug) or optionally, a set of related variables. In other embodiments, the data for one variable can be displayed on multiple lines. A set of these lines is displayed on the screen or page according to the granted permissions. These lines may be synchronized, such that all have the same starting time, ending time, and time scale. This allows the user to see correlations and other relationships across data. In prior systems, these correlations and relationships were difficult or impossible to understand as they come from data that are provided by different sources and which is stored separately.

The timeline display has three levels of control with fine granularity under the control of the owner: 1) sending and accepting an invitation, 2) choosing the access type (none or private, read, or edit) at the group level and down to the module (category or type) level, and 3) permissions down to the module level. Furthermore, the user (e.g., guest) has controls at the timeline level in selecting the timeframe for display and at the module level for example, list or graph view, and menus for filtering, view by, color by, and sort by. In certain embodiments, data filtering includes options such as filtering sensitive data, data entered by clinicians versus non-clinicians, and/or data entered by a specific user, for example. Other filtering options can be used in other embodiments. In certain embodiments, the “sort by” drop-downs for a module can include options for name, date, ascending or descending, for example. The “view by” drop-downs for a module can include options for average, minimum or maximum, for example. The “color by” drop-downs for a module can include options for coloring cells depending on various quantities represented in that cell, for example.

Therefore, the owner controls who can access their data, the type of access, and permissions all down to a category or module level of granularity. Modules for which the user does not have permission to at least view can display a statement that the user does not have permission for view the module data.

In certain embodiments, the display screen presents clear information about the medications the patient is taking as well as modifications to the medication regime. These screens illustrate a way to better assimilate and view and analyze complex drug regimes and their relative changes over time.

It may be noted that in these embodiments, the dates of starting a medication, and in some embodiments, stopping the medication are shown and correlated with one or more of the medication name, amount, and modification. This makes reading, comprehending, and judging the medication data significantly easier and quicker to undertake. This may allow not just specialists (such as rare and expensive clinical pharmacologists) to view and comprehend the data, but also physicians, clinicians, junior staff, researchers, caregivers, patients and their families and any other invited and permitted party.

In some embodiments, a step of displaying a time line or other graphic, showing data including but not limited to patients' ages, genders, locations, jobs, lifestyle choices, life events, underlying health conditions including pre-existing conditions, genetic identifiers, symptoms, treatments, drugs, or other health-related variable is provided. In other embodiments, the data includes clinical data along with lifestyle data, psycho-social data, environmental data and genetic data. This step can be useful in assessing medication or treatment success across the above-mentioned patient variables, discovering contraindications, or otherwise assessing medication efficacy and/or patient response.

The example timeline screen display illustrates a summary page of medical information for a particular patient, which may be plotted against patient background/symptom information using temporal correlation. Advantages of such a display include, without limitation, enriching clinical understanding of the patient history, reducing oversights and mistakes, and improving health outcomes and patient engagement. FIG. 52 can include an easy to understand graphic analysis of a patient's response to a possible drug or change in their drug regime. Such a graphic analysis helps to identify possible contraindications and the likely source of adverse effects.

Alternative forms of health treatment can be more easily compared to conventional treatment regimes, either for an individual patient as illustrated in FIG. 52 or on a population health basis (not shown). Other areas for optimization made possible by the system and method include, but are not limited to, optimization of supplements and nutrition, life events, lifestyle, traditional, complementary and alternative medicine, treatment regimes, patient inputs and feedback and other health professionals inputs and feedback.

Various data sets (e.g., lab tests, medications, vital signs, symptoms, and so forth) are rendered onto a single page summary for each particular patient. The data sets may be graphically summarized with data sourced from different parties shown in a different color or other indicia.

Even if the timeline page is larger than a screen size, the user need only scroll to display all of the desired data, rather than having to navigate additional links to other web pages with the associated delay and difficulty of adjacent viewing. Preferably, all of the above mentioned modules or categories of medical information are provided on the page, although in some cases, a subset of such data may be presented.

The system and method highlight issues which may not be easily visible in dispersed data sets. An easier-to-understand display assists with quicker and more comprehensive understanding of patient medication regimes, vitals, lab results, signs and symptoms, and other background health data.

An advantage of the system and method is that all members of a healthcare team (multi-disciplinary care team) having appropriate permissions, including patient and caregivers (e.g., family and other primary caregivers), as well as automatic medical data feeds can all input their respective data and still have it collated and displayed on the same single summary page. This capability fundamentally alters the ability of all members of that care team to view the patient condition holistically, with reference to all data, across any time scale.

The system and method has the capability to act as a collaborative tool for the multi-disciplinary care team including enabling the patient or their family to be a part of the care team, alerting them to important risks and changes to the patient's situation.

Through patient consent, multiple members of the patient's care team may be invited to the patient record, allowing all authorized team members to view a part or all of the patient's medical status (depending on the level of permissions granted). This invitation functionality, combined with the ability of the system and method to render and display data inputs of all the care team members on a single page, significantly increases collaboration and effectiveness of the care team, both for individual decision-making and through collaboration.

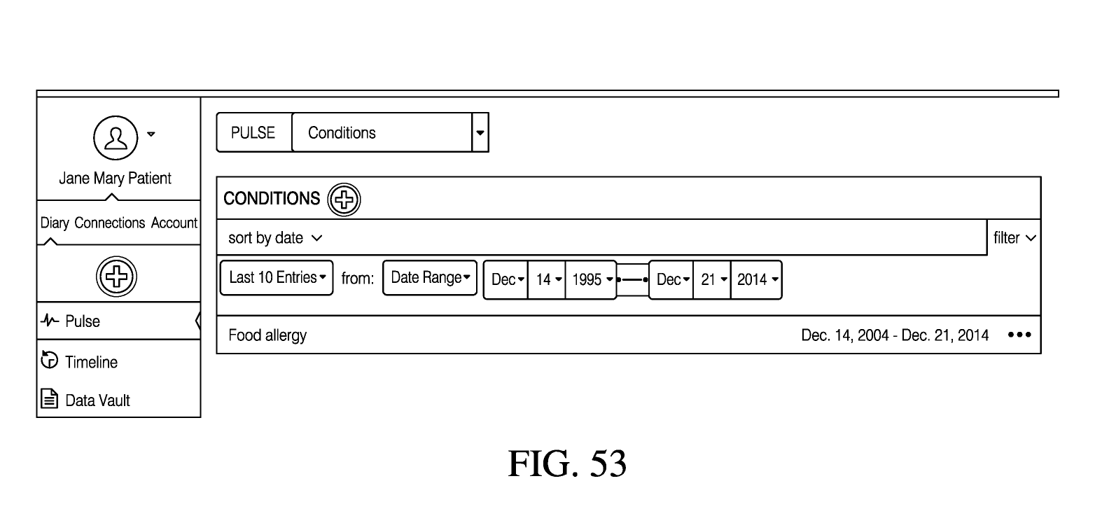

FIG. 53 is an example of a screen display illustrating an interface for displaying a page for each module which can be used to work with that module's data. Each module has a corresponding page which can be used to work with that module's data, e.g., adding new entries, locating entries via filters, deleting or modifying prior entries.

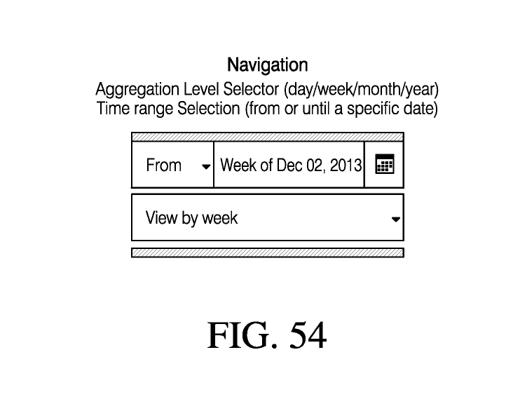

FIG. 54 is an example of a screen display illustrating a portion of an interface screen seen by a user for navigating a timeline of an owner. In certain embodiments, this portion allows a user to select an aggregation level from among day, week, month or year, and a starting date or ending date corresponding with the aggregation level selection. Other aggregation levels can be used in other embodiments.

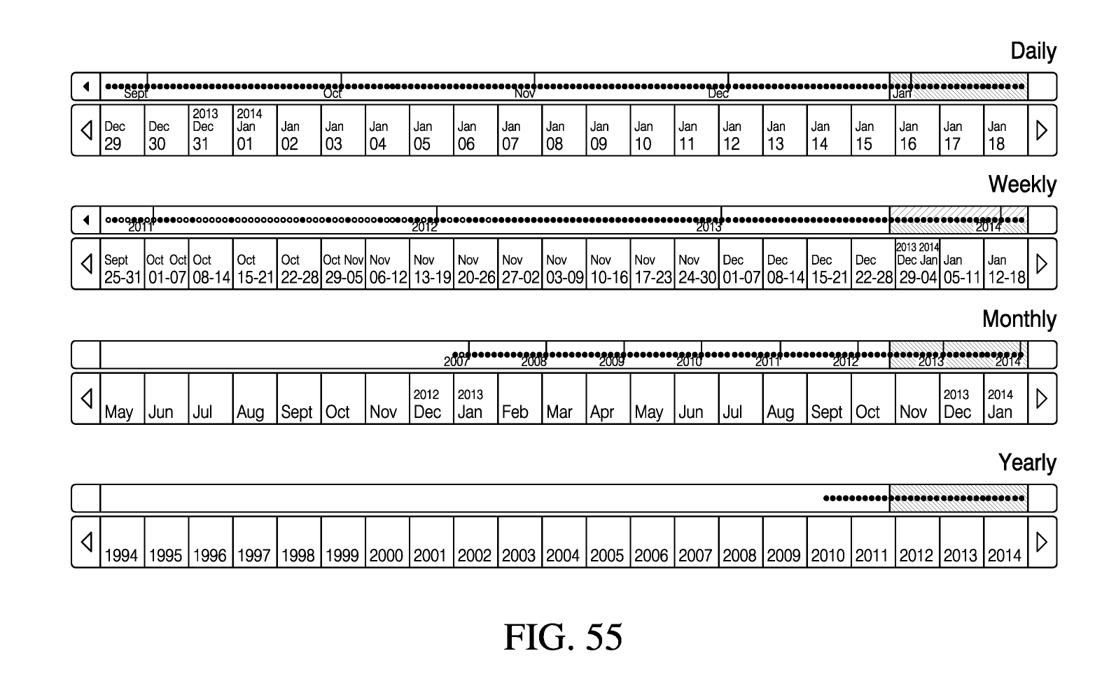

FIG. 55 is an example of a screen display illustrating a calendar bar portion of an interface screen for a timeline showing examples of possible aggregations levels. After receiving a request for a timeline display including an aggregation level selection from among day, week, month or year, and a starting date or ending date corresponding with the aggregation level selection, the system renders the calendar bar. This can include rendering the calendar bar having two row portions according to the received request, such that the bottom row portion displays a series of blocks with dates (e.g., December 29 for a daily level of aggregation, September 25-31 for weekly, May for monthly and 1994 for yearly) according to the starting date or ending date corresponding with the aggregation level selection. This can further include rendering a top row portion to display a series of dots at the selected aggregation level, where a light dot represents a lack of owner data for a time period at the selected aggregation level and a dark dot represents the presence of owner data for the time period (e.g., daily, weekly, monthly or yearly). At certain levels of aggregation either corresponding months or years are also displayed with the dots to identify the timeframes for the presence or absence of data, e.g., for a daily level of aggregation, a set of months are displayed, and for the weekly or monthly level, a set of years are displayed. The rendering can further include rendering so that the top row portion of the calendar bar displays a highlighted block over the dots corresponding to the dates in the bottom row portion, and such that if the cursor is moved to hover over another portion of the top row portion another highlighted block is displayed corresponding to the position of the hovering cursor, so that if a click (e.g., using a mouse or pad) event is received at the position of the hovering cursor, the timeline displays data corresponding to the date of the click and according to the selected aggregation level. The diaries support entry of exact time of day for data events. Therefore, in other embodiments, the timeline can display data items at an hourly or other portion of a day timescale.

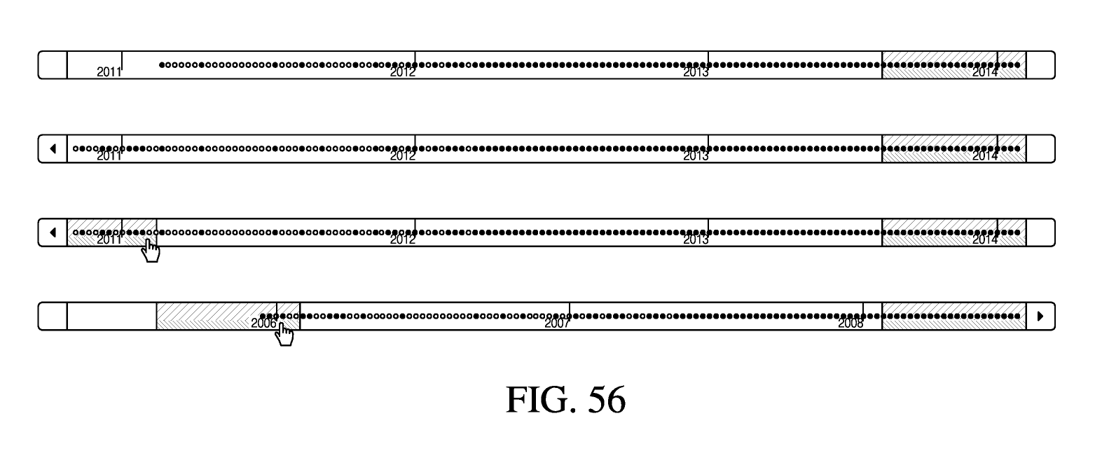

FIG. 56 is an example of a screen display illustrating a portion of an interface screen for a timeline showing examples of timeline data maps at a weekly level of aggregation. The data maps display a series of dots at the selected aggregation level, where a light dot represents a lack of owner data for a time period at the selected aggregation level and a dark dot represents the presence of owner data for the time period. In this example, corresponding years are also displayed with the dots to identify the timeframes for the presence or absence of data. The bottom two examples of timeline data maps illustrate a hand icon (representing the cursor) to hover over another portion of the data map and another highlighted block is displayed corresponding to the position of the hovering cursor, so that if a click event is received at the position of the hovering cursor, the timeline displays data corresponding to the date of the click and according to the selected aggregation level.

### Version Tracking

The system 100 tracks a version identifier of each owner's data. When a user has write permission for certain types or categories of data, the system records various information. In certain embodiments, who has edited what data, and what that edit was, and these changes from all authorized users are all permanently recorded in an editing/version tracking system in each diary. These changes are visible to each user of a particular owner's diary assuming they have the right level of permission to view the data. Further, those users with write ability for editing can institute further edits and have the result similarly logged for all authorized users to see. In certain embodiments, the core database provides functionality to verify whether the modified entity is currently the latest version. If it has been modified since it was fetched, the user can be notified and the save disallowed.

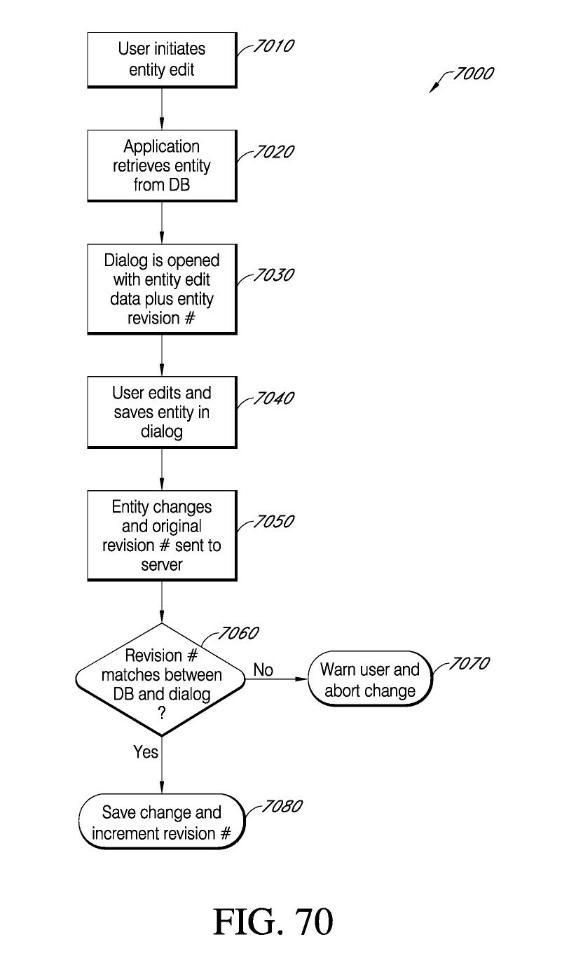

FIG. 70 is a flowchart illustrating an embodiment of a process 7000 for editing and version tracking. In certain embodiments, the process can be performed on the system 100 illustrated in FIG. 2A. This process 7000 is followed when a user, e.g., the owner of the data, a guest or a staff member of an organization such as a doctor, adds to or edits any portion of diary data for an owner.

The editing/version tracking is supported in the core database, but it is handled by the user interface for various reasons including maintenance of version identification during the editing process, and presentation of related warnings/errors to the user.

Beginning at state 7010, a user with appropriate write permissions initiates editing of an entity of the owner's data. Proceeding to state 7020, process 7000 retrieves the entity from the core database. The version number and all other data (e.g., date and time of revision, and identifier of the person performing the edit) are stored as part of the entity itself in the core database. In certain embodiments, the entity can be any data item in the database, such as for example, module (category) data. All that needs to be done to make an entity versionable is to add the required columns in the core database. The build system recognizes those fields are present and introduces versioning support for that entity from that point forward. In certain embodiments, the version number is per entity.

Continuing at state 7030, a dialog box or window is opened with entity edit data along with an entity revision number. At state 7040, the user edits (e.g., modifies, deletes portions or adds data), and saves the entity in the dialog. Moving to state 7050, the entity changes and original revision number are received at the server. Proceeding to a decision state 7060, process 7000 determines if the revision number matches between the core database and the dialog. If not, process 7000 moves to state 7070 to warn the user that the entity (of the owner's data) has been modified by another user since it was fetched by the first user, and aborts the change. However, if the revision number matches between the core database and the dialog as determined at decision state 7060, process 7000 advances to state 7080 to save the change and increments the revision number.

As a result of storing the edit-related data with the entity, the system can display a change history for all previous revisions of that data to any user having the appropriate read permission for the category of data corresponding to the entity item.

### Widgets

A widget is a major component in the system that can be shown on a display screen or hidden at the user's option and can be used to refer to components on both the timeline and the pulse pages. In certain embodiments, widgets provide a predefined list of templated views (data combinations) for the user to choose from e.g., diabetes, weight loss, and hypertension. In general within this application, a widget is a user interface component that can be added to or removed from a page. As previously described, in certain embodiments, the pulse page is a dashboard for an owner's diary, which allows users to place the important aspects of their diary on one page. This allows owners and guests to see at a glance what is relevant to them without having to navigate throughout the application. Widgets allow a user to save multiple views that are specific to their own needs and can be added to their views from a widget library. The user can add, order and remove widgets to each of their views as desired.

In certain embodiments there is a predefined set of widgets that can render out each of the modules contained within a diary. Basic widget types include comparable date-time data widgets (e.g., exercise log, medication log), non-comparable date-time data widgets (e.g., life events, notes), time range data widgets (e.g., illnesses), and avatar widgets. Advanced module specific widgets can be developed and added at a later time (e.g., drug regimes). In addition, compound widgets (e.g., widgets that display multiple data sources together) can be developed and added at a later time. The system provides an ability to configure the display mode of each widget (e.g., linegraph, bargraph, or data). The system also provides an ability to configure sub-dimensions of data to show intensity and/or duration, for example. In certain embodiments, a default set of widgets is provided for the user. An example of a default pulse page can contain widgets for weight, latest new diary entries, latest user activity, latest medication taken, and latest physical activity.

Widgets can have data filtering options such as filtering sensitive data, data entered by clinicians versus non-clinicians, and/or data entered by a specific user, for example. Other filtering options can be used in other embodiments.

Any given viewer of the timeline only has access to the underlying data that they have been granted permission to see. This means that although a widget can be added, the underlying data for the widget may not be available to the particular user.

The pulse page can have various predefined widgets that a user can add to their page. Users are able to remove widgets from the page. In certain embodiments, widgets can be sorted via a drag and drop operation or via dragging in a mobile device environment. The selected widgets and order of widgets can be saved against a particular owner (patient).

Several example widgets will now be described. A Medication Taken widget can include options for heading (a custom widget heading), medication (choose a medication), focus on (e.g., amount taken, time taken), and timespan (today, last seven days, last thirty days, last six months). A Physical Activity widget can include options for heading (a custom widget heading), physical activity (choose an activity), focus on (e.g., duration, time of activity) and timespan (today, last seven days, last thirty days, last six months). A Body Measurements widget can include options for heading (a custom widget heading), measurement (choose measurement), focus on (e.g., daily average), and timespan (last seven days, last thirty days, last six months). A Vitals widget can include options for heading (a custom widget heading), vital (choose one vital), focus on (e.g., average), and timespan (today, last seven days, last thirty days, last six months). A Nutrition widget can include options for heading (a custom widget heading), focus on (e.g., total calories, total carbs, total cholesterol, total fiber, total sugar, total protein, total fat, total saturated fat, total sodium), and timespan (today, last seven days, last thirty days, last six months). Other widgets can include Latest Entries, Sleep, Mood, Pain, and Access with corresponding options.

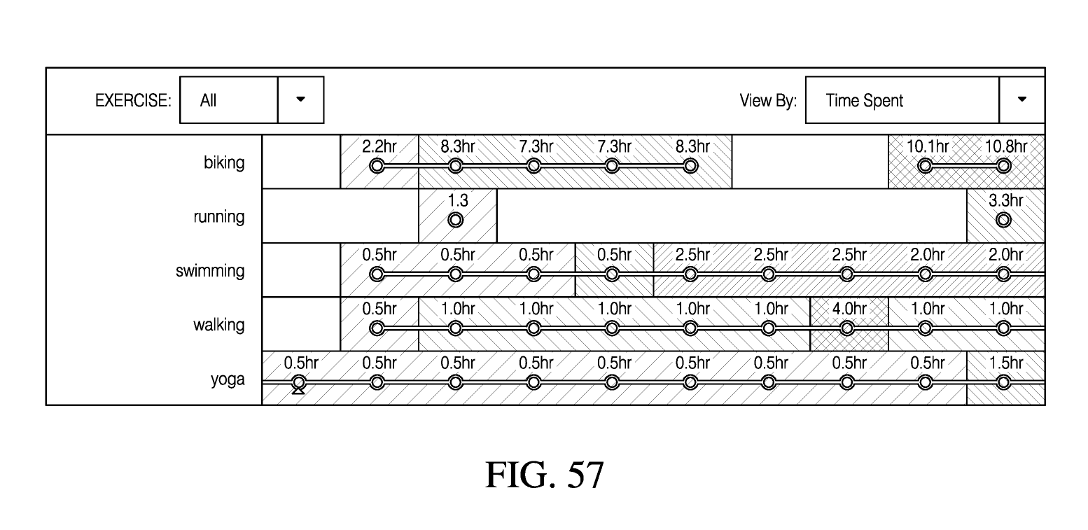

FIG. 57 is an example of a portion of a screen display illustrating an interface screen for an example widget for physical activity or exercise history showing a list view having two controls with drop-down menus for exercise type and view by. Each block portion can have a duration of time performing the exercise and utilize colors to indicate status based on goals, for example.

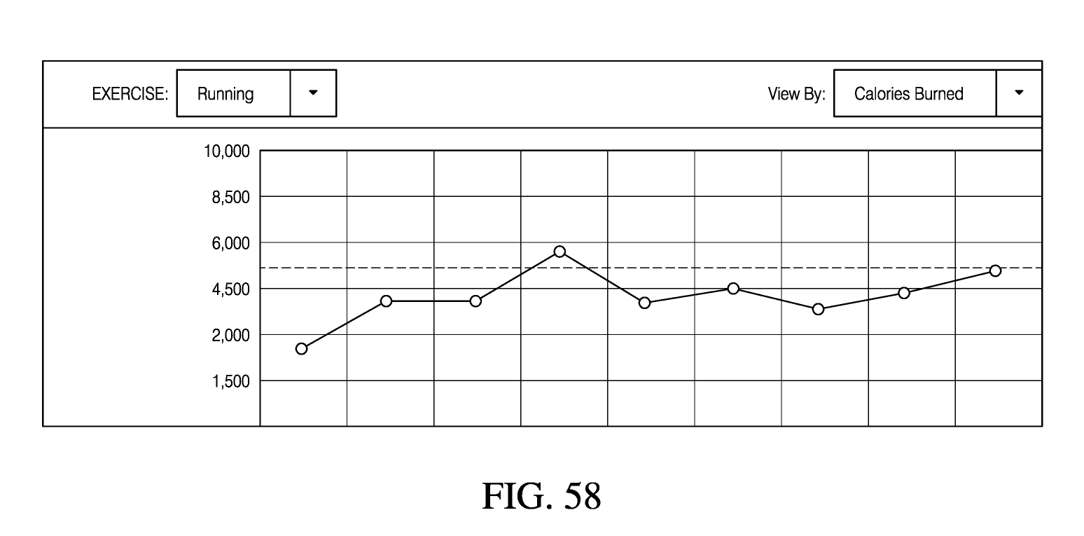

FIG. 58 is an example of a portion of a screen display illustrating an interface screen for an example widget showing a graphical data view responsive to the selected controls including a selected exercise type and view by calories burned.

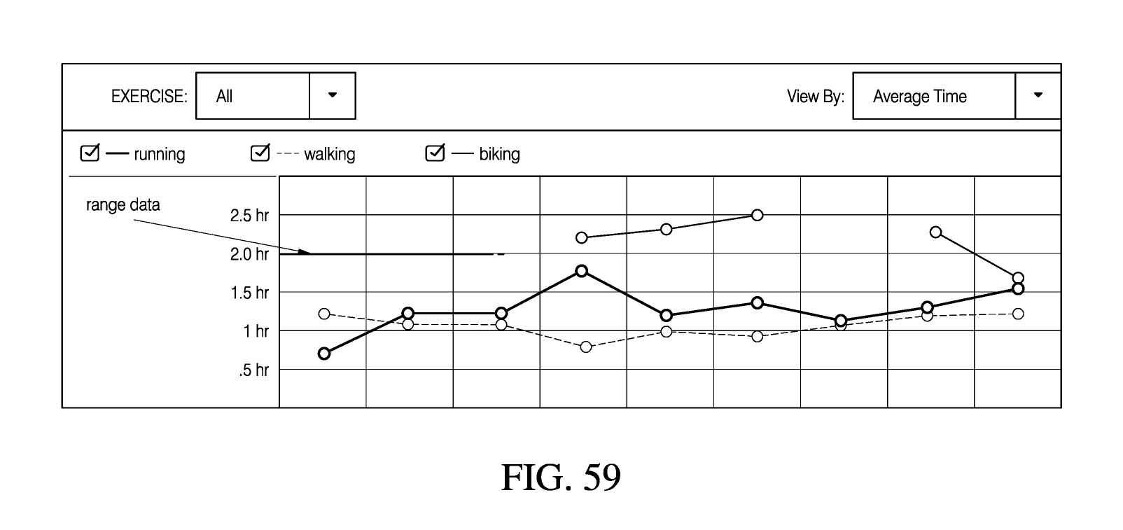

FIG. 59 is an example of a portion of a screen display illustrating an interface screen for an example widget showing a graphical data view responsive to the selected controls including all exercise types and view by average time.

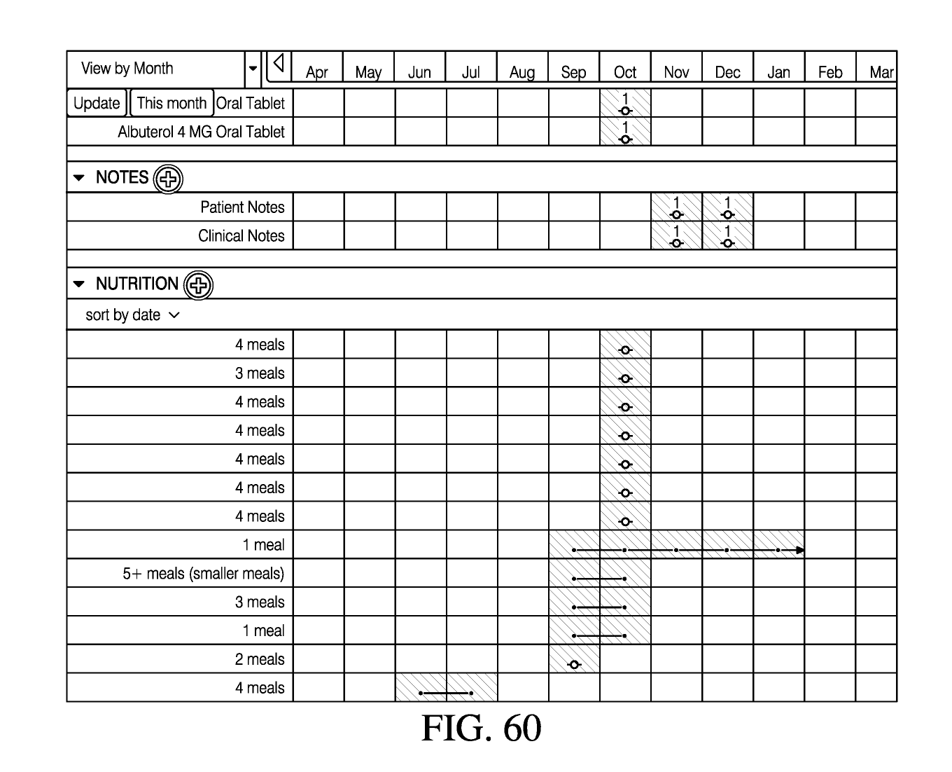

FIG. 60 is an example of a screen display illustrating an interface screen for several example widgets showing a list view responsive to the selected controls and where each widget can have different controls.

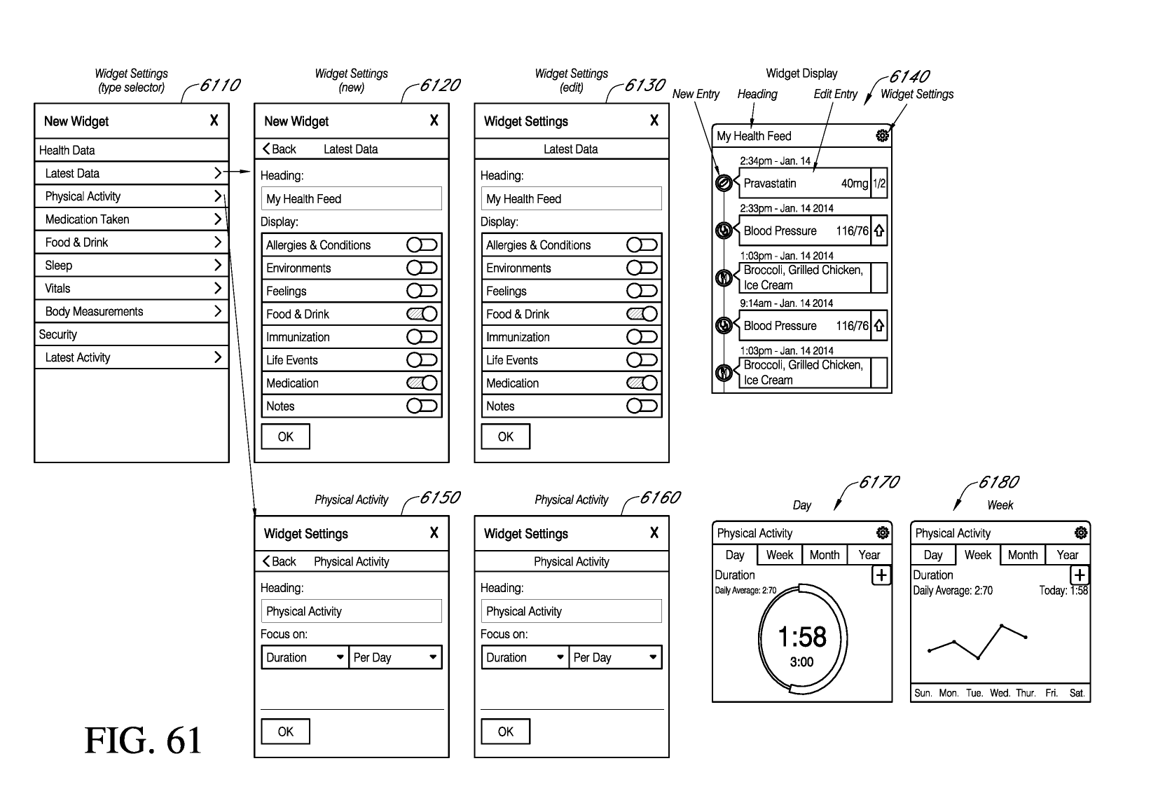

FIG. 61 is an example of screen displays illustrating interface screens for example widget settings and widget display for a my health feed widget, and example widget settings and corresponding widget displays for a physical activity widget. The screen displays are such as can be seen on a mobile computing device such as a smartphone, for example.

Display screen 6110 is an example dialog that appears when the user clicks the ‘edit.’ button on the pulse page, and the +(plus) button to add a new widget. Display screen 6120 is an example dialog that appears to allow the user to set up the new widget that is chosen (in this case Health Data—Latest Data). Display screen 6130 is an example of a dialog that appears and allows the user to change the settings for a pulse page widget (usually the same as display screen 6120). Display screen 6140 is an example of an actual widget as it appears on the pulse page.

Display screens 6150 to 6180 illustrate a widget for physical activity. Display screen 6150 is an example dialog that appears to allow the user to set up the new widget that is chosen (in this case Health Data—Physical Activity). Display screen 6160 is an example of a dialog that appears and allows the user to change the settings for a pulse page widget (usually the same as display screen 6150). Display screen 6170 is an example of an actual widget as it appears on the pulse page for a day of physical activity, and display screen 6180 is for a week of physical activity.

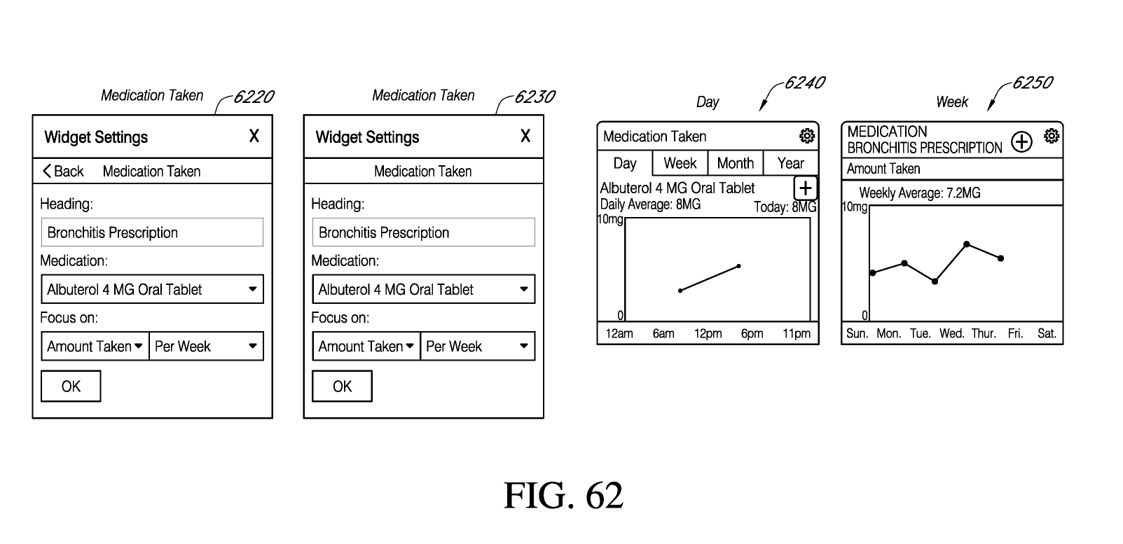

FIG. 62 is an example of screen displays illustrating interface screens for example widget settings and corresponding widget displays for a medication taken widget. Display screen 6220 is an example dialog that appears to allow the user to set up the new widget that is chosen (in this case Health Data—Medication Taken). Display screen 6230 is an example of a dialog that appears and allows the user to change the settings for a pulse page widget. Display screen 6240 is an example of an actual widget as it appears on the pulse page for a day of medication taken, and display screen 6250 is for a week of medication taken.

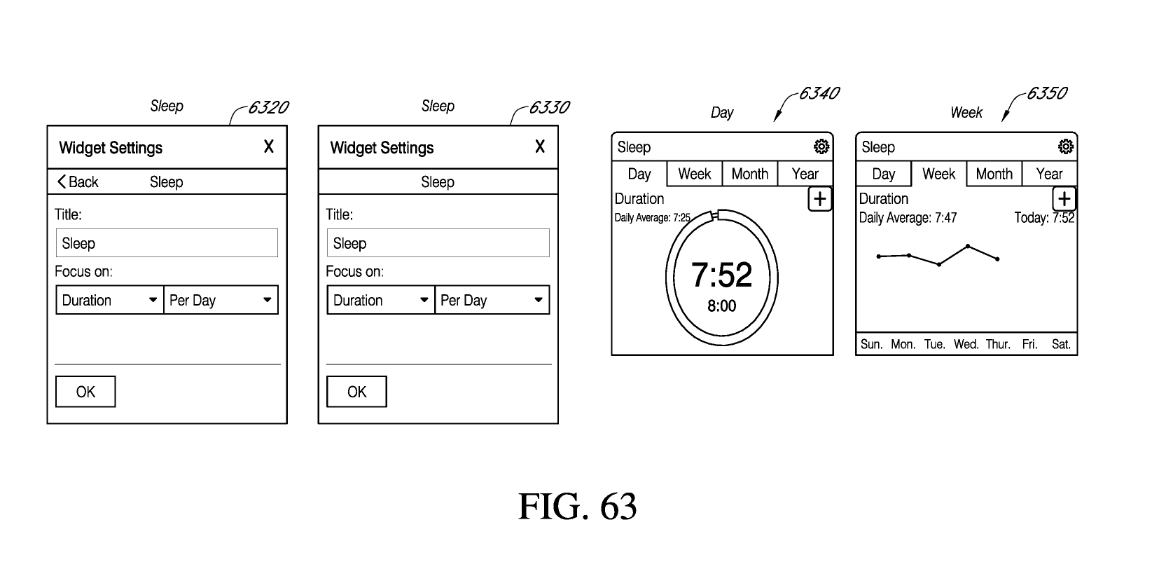

FIG. 63 is an example of screen displays illustrating interface screens for example widget settings and corresponding widget displays for a sleep widget. Display screen 6320 is an example dialog that appears to allow the user to set up the new widget that is chosen (in this case Health Data—Sleep). Display screen 6330 is an example of a dialog that appears and allows the user to change the settings for a pulse page widget. Display screen 6340 is an example of an actual widget as it appears on the pulse page for a day of sleep data, and display screen 6350 is for a week of sleep data.

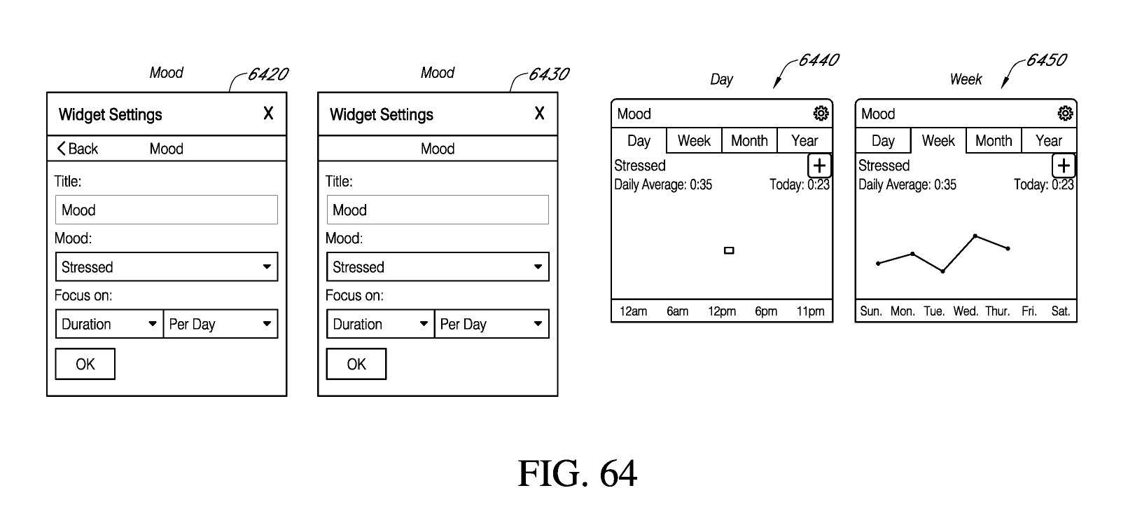

FIG. 64 is an example of screen displays illustrating interface screens for example widget settings and corresponding widget displays for a mood widget. Display screen 6420 is an example dialog that appears to allow the user to set up the new widget that is chosen (in this case Mood). Display screen 6430 is an example of a dialog that appears and allows the user to change the settings for a pulse page widget. Display screen 6440 is an example of an actual widget as it appears on the pulse page for a day of mood data, and display screen 6450 is for a week of mood data.

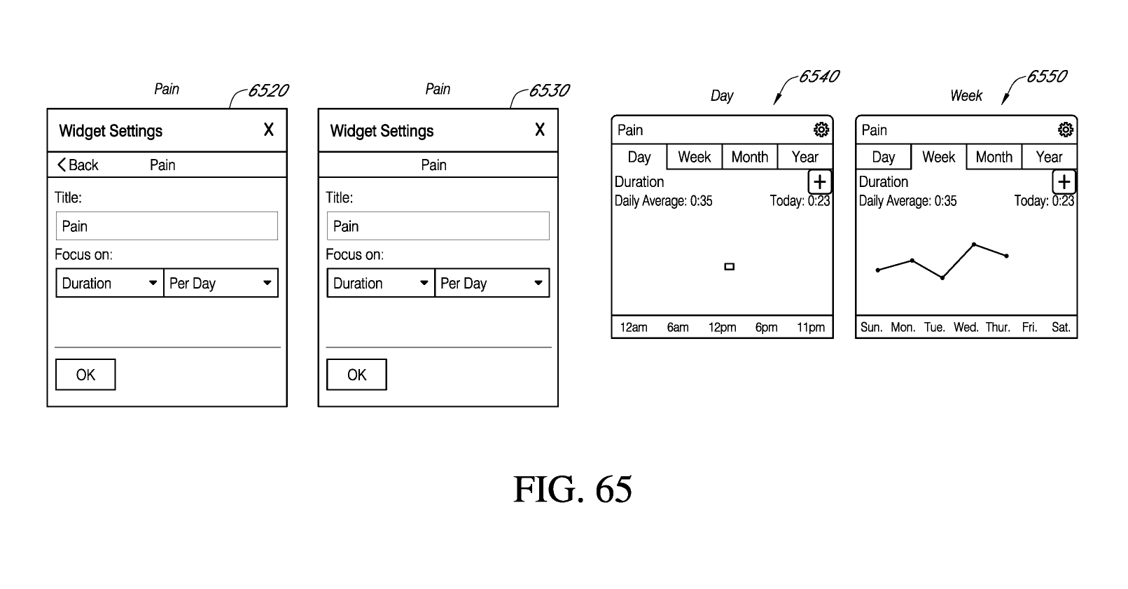

FIG. 65 is an example of screen displays illustrating interface screens for example widget settings and corresponding widget displays for a pain widget. Display screen 6520 is an example dialog that appears to allow the user to set up the new widget that is chosen (in this case Pain). Display screen 6530 is an example of a dialog that appears and allows the user to change the settings for a pulse page widget. Display screen 6540 is an example of an actual widget as it appears on the pulse page for a day of pain data, and display screen 6550 is for a week of pain data.

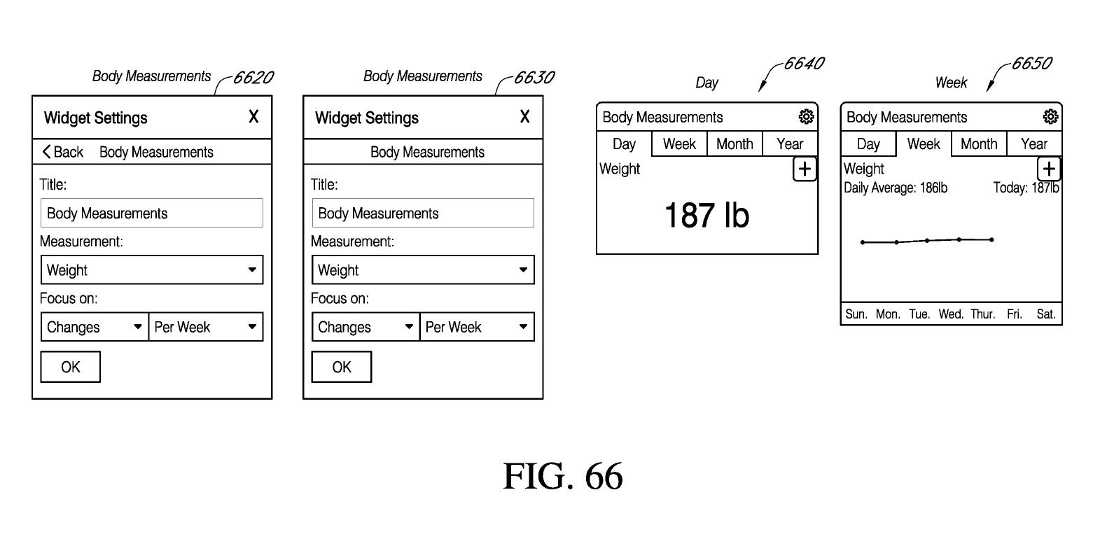

FIG. 66 is an example of screen displays illustrating interface screens for example widget settings and corresponding widget displays for a body measurements widget. Display screen 6620 is an example dialog that appears to allow the user to set up the new widget that is chosen (in this case Health Data—Body Measurements). Display screen 6630 is an example of a dialog that appears and allows the user to change the settings for a pulse page widget. Display screen 6640 is an example of an actual widget as it appears on the pulse page for a day of body measurements data, and display screen 6650 is for a week of body measurements data.

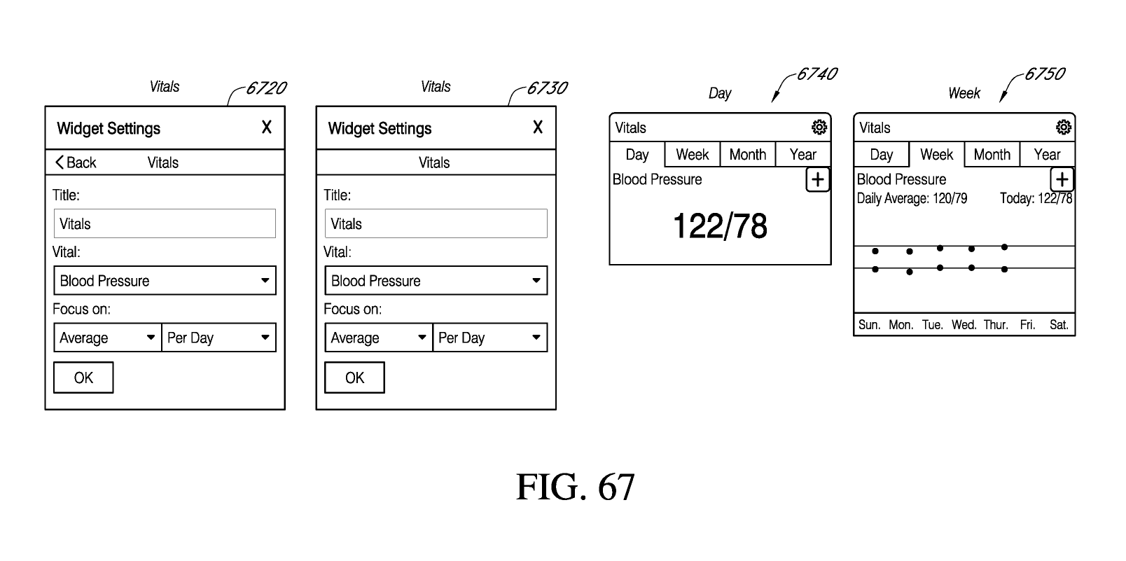

FIG. 67 is an example of screen displays illustrating interface screens for example widget settings and corresponding widget displays for a vitals widget. Display screen 6720 is an example dialog that appears to allow the user to set up the new widget that is chosen (in this case Health Data—Vitals). Display screen 6730 is an example of a dialog that appears and allows the user to change the settings for a pulse page widget. Display screen 6740 is an example of an actual widget as it appears on the pulse page for a day of vitals data, and display screen 6750 is for a week of vitals data.

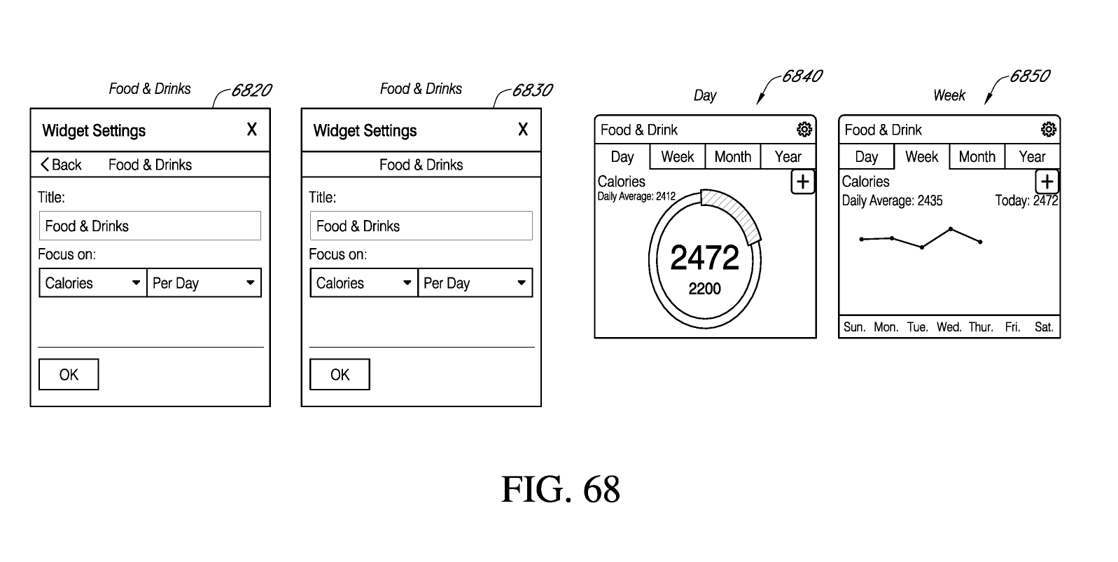

FIG. 68 is an example of screen displays illustrating interface screens for example widget settings and corresponding widget displays for a food and drinks widget. Display screen 6820 is an example dialog that appears to allow the user to set up the new widget that is chosen (in this case Health Data—Food & Drinks). Display screen 6830 is an example of a dialog that appears and allows the user to change the settings for a pulse page widget. Display screen 6840 is an example of an actual widget as it appears on the pulse page for a day of food & drinks data, and display screen 6850 is for a week of food & drinks data.

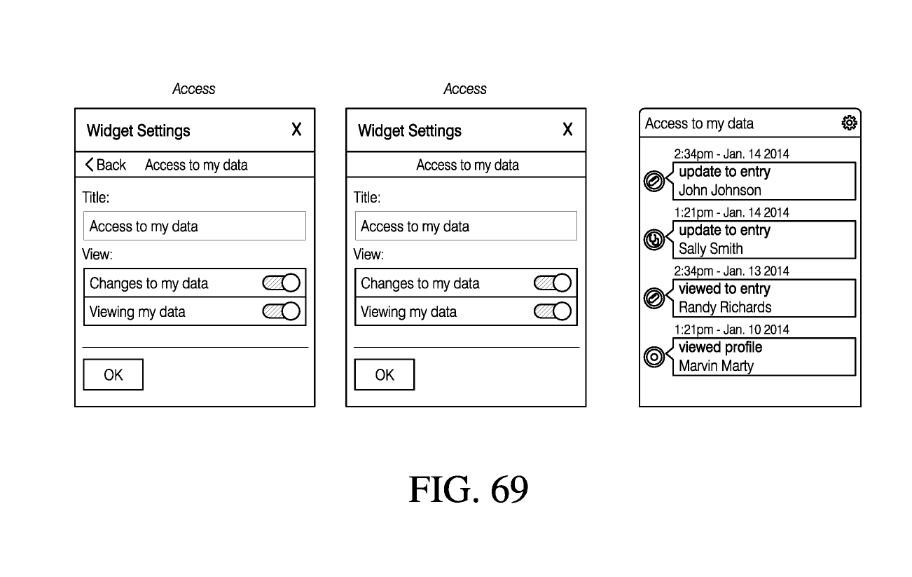

FIG. 69 is an example of screen displays illustrating interface screens for example widget settings and a corresponding widget display for an access widget. Display screen 6920 is an example dialog that appears to allow the user to set up the new widget that is chosen (in this case Access). Display screen 6930 is an example of a dialog that appears and allows the user to change the settings for a pulse page widget. Display screen 6940 is an example of an actual widget as it appears on the pulse page listing items of access to the owner's data.

### CONCLUSION

The overall effect of the system and method is to control access and access type to various parties associated with the data owner so as to, for example, preserve privacy, reduce oversights, omissions and mistakes, as well as allow a health professional to more comprehensively diagnose, and in less time.

Various illustrative logics, logical blocks, modules, circuits and algorithm steps described in connection with the implementations disclosed herein may be implemented as electronic hardware, computer software, or combinations of both. The interchangeability of hardware and software has been described generally, in terms of functionality, and illustrated in the various illustrative components, blocks, modules, circuits and steps described above. Whether such functionality is implemented in hardware or software depends upon the particular application and design constraints imposed on the overall system.

In one or more aspects, the functions described may be implemented in hardware, digital electronic circuitry, computer software, firmware, including the structures disclosed in this specification and their structural equivalents thereof, or in any combination thereof. Implementations of the subject matter described in this specification also can be implemented as one or more computer programs, e.g., one or more modules of computer program instructions, encoded on a computer storage media for execution by, or to control the operation of, data processing apparatus.

If implemented in software, the functions may be stored on or transmitted over as one or more instructions or code on a computer-readable medium. The steps of a method or algorithm disclosed herein may be implemented in a processor-executable software module which may reside on a computer-readable medium. Computer-readable media includes both computer storage media and communication media including any medium that can be enabled to transfer a computer program from one place to another. A storage media may be any available media that may be accessed by a computer. By way of example, and not limitation, such computer-readable media may include RAM, ROM, EEPROM, CD-ROM or other optical disk storage, magnetic disk storage or other magnetic storage devices, or any other medium that may be used to store desired program code in the form of instructions or data structures and that may be accessed by a computer. Also, any connection can be properly termed a computer-readable medium. Disk and disc, as used herein, includes compact disc (CD), laser disc, optical disc, digital versatile disc (DVD), floppy disk, and Blu-ray disc where disks usually reproduce data magnetically, while discs reproduce data optically with lasers. Combinations of the above should also be included within the scope of computer-readable media. Additionally, the operations of a method or algorithm may reside as one or any combination or set of codes and instructions on a machine readable medium and computer-readable medium, which may be incorporated into a computer program product.

Certain features that are described in this specification in the context of separate implementations also can be implemented in combination in a single implementation. Conversely, various features that are described in the context of a single implementation also can be implemented in multiple implementations separately or in any suitable subcombination. Moreover, although features may be described above as acting in certain combinations and even initially claimed as such, one or more features from a claimed combination can in some cases be excised from the combination, and the claimed combination may be directed to a subcombination or variation of a subcombination.

Similarly, while operations are depicted in the drawings in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system components in the implementations described above should not be understood as requiring such separation in all implementations, and it should be understood that the described program components and systems can generally be integrated together in a single software product or packaged into multiple software products.

The foregoing description details certain embodiments of the invention. It will be appreciated, however, that no matter how detailed the foregoing appears in text, the invention can be practiced in many ways. As is also stated above, it should be noted that the use of particular terminology when describing certain features or aspects of the invention should not be taken to imply that the terminology is being re-defined herein to be restricted to including any specific characteristics of the features or aspects of the invention with which that terminology is associated. The scope of the invention should therefore be construed in accordance with the appended claims and any equivalents thereof.

1. A method of controlling the granting of permissions to selected recipients by an owner of data in a system including at least a plurality of computing devices, a server, a network and a database, the method comprising:
receiving an electronic authentication at the server from an owner of data stored in an owner's electronic diary portion of a database;
receiving at the server an electronic address corresponding to each of one or more desired recipients to be invited to access specific categories of data controlled by the owner;
creating an electronic record in the database for each of the desired recipients;
identifying a particular combination of permissions that the one or more recipients will have if the invitation is accepted by the recipient and is subsequently granted, wherein a permission provides an ability to perform one or more specific actions;
storing the particular permissions in the record corresponding to each of the one or more recipients along with an identifier of the recipient;
sending, via a computer network, an electronic communication to the one or more desired recipients, wherein each electronic communication includes a unique uniform resource locator for use to reply to the communication;
receiving, via a computer network, an electronic message including an acceptance or rejection of the invitation from each of the one or more recipients;
if the received electronic message includes acceptance of a particular recipient's invitation:
sending to the owner, via a computer network, an acceptance notification corresponding with the particular recipient's invitation;
receiving at the server from the owner a message having an electronic address corresponding to the particular recipient, wherein the message includes an indication of either a grant of the particular recipient's accepted invitation or a cancellation of the particular recipient's accepted invitation; and
storing in a record corresponding to the particular recipient, an indicator of the acceptance or rejection by the recipient of the corresponding invitation, an indicator of cancellation if the invitation is canceled by the owner, and an indicator of acceptance if the invitation is granted by the owner so as to facilitate owner-controlled electronic access to the owner's data.

2-7. (canceled)

8. The method of claim 1, additionally comprising receiving an electronic request from the owner to add or remove permissions without the agreement of a particular recipient.

9. The method of claim 1, additionally comprising usage of the granted permissions by the selected recipients comprising:
receiving a request for a graphical display of owner data from a particular one of the recipients;
determining cumulative permissions for the particular recipient, wherein if the particular recipient is a part of an organization, the determining further comprises determining if the particular recipient is associated with an access group shared with the owner;
retrieving data identified by the cumulative permissions in the owner's electronic diary; and
generating an HTML-based screen display of the retrieved data.

10-16. (canceled)

17. The method of claim 9, additionally comprising:
receiving a request for a timeline display including an aggregation level selection from among day, week, month or year, and a starting date or ending date corresponding with the aggregation level selection; and
rendering a single horizontal calendar bar having two row portions according to the received request, wherein the bottom row portion displays a series of blocks with dates according to the starting date or ending date corresponding with the aggregation level selection, and wherein the top row portion displays a series of dots at the selected aggregation level, where a light dot represents a lack of owner data for a time period at the selected aggregation level and a dark dot represents the presence of owner data for the time period.

18. The method of claim 17, wherein the top row portion of the calendar bar further displays a highlighted block over the dots corresponding to the dates in the bottom row portion, and wherein if a cursor is moved to hover over another portion of the top row portion another highlighted block is displayed corresponding to the position of the hovering cursor, wherein if a click event is received at the position of the hovering cursor, the timeline displays data corresponding to the date of the click and according to the selected aggregation level, and wherein the dots are displayed at evenly spaced intervals according to the selected aggregation level.

19-24. (canceled)

25. The method of claim 1, wherein permission status includes given, received and accepted, and wherein permission kinds include read, write and no access.

26-27. (canceled)

28. A method of controlling the granting of permissions to selected recipients by an owner of data in a system including at least a plurality of computing devices, a server and a network, the method comprising:
identifying permissions that an organization has if an invitation from a particular owner of the data is accepted by an administrator of the organization, wherein a permission provides an ability to perform one or more specific actions;
sending, via a computer network, an electronic communication comprising at least the invitation, the combination of permissions to the administrator of the organization and a unique uniform resource locator for use in replying to the electronic communication;
receiving an assignment of the particular owner to an organization-defined access group;
receiving a mapping of the particular owner and one or more selected staff members of the organization to the access group to link the particular owner and the selected staff members;
receiving an identification of at least one role corresponding to the staff members of the organization;
receiving an assignment of the one or more selected staff members of the organization to the at least one role;
granting particular permissions to the selected staff members for the particular owner according to the particular access group having the particular owner and the selected staff members, and according to the at least one role having the selected staff; and
storing in a data structure an indicator of the acceptance or rejection of the invitation, an identifier of each of the selected staff members and a corresponding combination of permissions granted to each of the selected staff members so as to facilitate owner-controlled electronic access to the owner's data.

29. The method of claim 28, wherein a particular staff member can access an owner's diary only if the owner and the staff member share a common access group.

30. The method of claim 28, wherein a particular staff member accesses an owner's diary based on the cumulative permissions from all roles the staff member belongs to.

31. The method of claim 28, wherein the particular permissions are restricted to those given to the organization from the owner's account.

32. The method of claim 28, wherein a role is an organization defined combination of permissions.

33. The method of claim 28, additionally comprising usage of the granted permissions by the selected staff members comprising:
receiving a request for a graphical display of owner data from a particular one of the staff members, wherein the data is grouped among multiple categories;
determining cumulative permissions for the particular staff member, wherein the determining further comprises determining if the particular staff member is associated with an access group shared with the owner;
retrieving data-identified by the cumulative permissions in the owner's electronic diary; and
generating an HTML-based screen display of the retrieved data.

34. The method of claim 33, additionally comprising receiving a navigational request from the particular staff member to manipulate the HTML-based screen display.

35. The method of claim 28, wherein the staff member is a user who belongs to the organization, including one of a doctor, a nurse, a technician, a pharmacist, and an administrator.

36. The method of claim 28, wherein permission kinds include read, write and no access.

37. The method of claim 28, wherein the roles additionally include selected categories of data that corresponding staff members have access to, wherein the categories of data include medical records, symptoms and/or vital signs, medications and/or laboratory results, emotional, life events, genetic markers and lifestyle.

38-44. (canceled)

45. The method of claim 33, wherein the screen display includes data for the categories for which the particular staff member has permission, and wherein the screen display indicates the categories for which the particular staff member does not have permissions.

46-49. (canceled)

50. The method of claim 45, wherein generating the HTML-based screen display includes applying one or more controls for each category corresponding to the granted permissions, wherein the controls for each permitted category are independent of the controls for the other permitted categories.

51-56. (canceled)

57. The method of claim 28, additionally comprising:
scanning a source document corresponding to a particular owner;
extracting medical data from the scanned document including a reference to the source document;
storing the source document in an electronic storage separately from a diary for the particular owner's data; and
storing the extracted medical data and the reference to the source document in the particular owner's diary based on a category of the extracted data, wherein the stored reference enables a user viewing the extracted data to navigate to and view the source document.

58-60. (canceled)

61. The method of claim 1, wherein the categories of data include clinical data, lifestyle data, psycho-social data, environmental data and genetic data.

62. A method of controlling the granting of permissions to selected recipients by an owner of data in a system including at least a plurality of computing devices, a server and a network, the method comprising:
identifying permissions that an organization has if an invitation from a particular owner of the data is accepted by an administrator of the organization, wherein a permission provides an ability to perform one or more specific actions;
sending, via a computer network, an electronic communication comprising at least the invitation, the combination of permissions to the administrator of the organization and a unique uniform resource locator for use in replying to the electronic communication;
receiving an assignment of the particular owner to an organization-defined access group;
receiving a mapping of the particular owner and one or more selected staff members of the organization to the access group to link the particular owner and the selected staff members;
receiving an assignment of the one or more selected staff members of the organization to at least one organizational role;
granting particular permissions to the selected staff members for the particular owner according to the particular access group having the particular owner and the selected staff members, and according to the at least one role having the selected staff;
storing in a data structure an indicator of the acceptance or rejection of the invitation, an identifier of each of the selected staff members and a corresponding combination of permissions granted to each of the selected staff members;
receiving edits to the owner's data or new data from a first user having corresponding granted write permissions;
tracking an identity of the first user, a time and date of the edits or new data, a type or category updated, and the edit or new data in a database; and
changing a version identifier for the updated owner's data.

63. The method of claim 62, additionally comprising:
determining whether the owner's data has been modified by a second user since it was fetched by the first user; and
notifying the first user that the update is disallowed.

64-65. (canceled)

66. The method of claim 62, wherein the update is visible to each subsequent user having corresponding granted permissions to access the owner's data having the changed version identifier.

67. The method of claim 62, additionally comprising displaying a change history for previous versions of the edited owner's data to a user having the appropriate read permission for the category of data corresponding to the edited data.

68-76. (canceled)
